(19) United States
(12) Patent Application Publication
(10) Pub. No.: US 2010/0249125 A1
(43) Pub. Date: Sep. 30, 2010

(54) INHIBITION OF PROSTGLANDIN E2 RECEPTORS FOR THE TREATMENT OF ENDOMETRIOSIS

(75) Inventors: Joe A. Arosh, College Station, TX (US); Sakhila K. Banu, College Station, TX (US)

Correspondence Address: CHALKER FLORES, LLP, 2711 LBJ FRWY, Suite 1036, DALLAS, TX, 75234, US

(73) Assignee: THE TEXAS A&M UNIVERSITY SYSTEM, College Station, TX (US)

(21) Appl. No.: 12/730,824

(22) Filed: Mar. 24, 2010

Related U.S. Application Data
(60) Provisional application No. 61/165,302, filed on Mar. 31, 2009.

(51) Int. Cl. A61K 31/535; A61K 31/35

(52) U.S. Cl. 514/231.2; 514/455

(57) ABSTRACT

The present invention describes the selective inhibition of prostaglandin receptors (PGE₂), e.g., EP2 and EP4, by one or more inhibitors are selected from 6-Isopropoxy-9-oxoxanthene-2-carboxylic acid and (4Z)-7-[(rel-1S,2S,5R)-5-((1,1′-Biphenyl-4-yl)methoxy)-2-(4-morpholinyl)-3-oxocyclopentyl]-4-heptenoic acid and salts thereof as a potential therapeutic tool or to expand the spectrum of currently available treatment options for endometriosis and other chronic gynecological diseases in reproductive age women. PGE₂ inhibition through novel cell signaling mechanisms identified by the present invention provides an effective and attractive therapeutic tool for treatment without compromising pregnancy and sex life in the women.

### CROSS-REFERENCE TO RELATED APPLICATIONS

This application claims benefit of U.S. Provisional Application Ser. No. 61/165,302 filed Mar. 31, 2009 which is incorporated herein by reference in its entirety.

### TECHNICAL FIELD OF THE INVENTION

The present invention relates in general to the field of chronic gynecological diseases and more specifically to a therapeutic approach involving selective inhibition of prostaglandin receptors, e.g., EP2 and EP4, as a potential therapeutic tool or to expand the spectrum of currently available treatment options for endometriosis in women.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with the treatment options for chronic gynecological diseases in reproductive age women and more specifically to the inhibition of prostaglandin receptors EP2 and EP4 as a treatment option for endometriosis in women.

Endometriosis is a benign chronic gynecological disease of reproductive age women characterized by the presence of functional endometrial tissues outside the uterine cavity. It is an estrogen-dependent disease. Current treatment modalities to inhibit biosynthesis and actions of estrogen compromise menstruation, pregnancy, reproductive health of women, and failure to prevent reoccurrence of disease. US Patent Publication No. 2009060997 (Seitz, et al., 2009) describes combination of an anti-androgenic gestagen at a daily dose of from an ovulation-inhibiting dose up to at most twice the ovulation-inhibiting dose and from 0.1 to 10 mg of (6S)-5-methyltetrahydrofolate are used to produce a pharmaceutical preparation for therapeutically treating endometriosis while simultaneously reducing therapy side effects including the negative effect on bone density and/or bone metabolism, reducing the risk of osteoporosis and, in the event of pregnancy, reducing the risk of congenital malformations.

Publication No. WO2006116873 (Bedaiwy et al., 2008) teaches the detection of endometriosis in individuals based on the presence of one or more polymorphism in a gene associated with the fibrinolytic pathway, and methods for treating or preventing endometriosis by modulating the fibrinolytic pathway.

Publication No. WO2008058514 (Werz and Siemoneit, 2008) discloses the use of preparations with pentacyclic triterpenoic acids and structural derivatives thereof, in particular boswellic acids from Boswellia species (such as B. serrata, B. carterii, B. sacra), for inhibiting the inducible mitochondrial prostaglandin E2 synthase-1. In addition, the invention relates to the use of pentacyclic triterpenoic acids and derivatives thereof for producing a medicament for the treatment of PGE₂-mediated diseases.

U.S. Pat. No. 5,744,464 (Elger et al., 1998) describes antigestagens which inhibit prostaglandin synthesis by the uterus and thus can be used to treat symptoms of dysmennhorea.

### SUMMARY OF THE INVENTION

The present invention describes the selective inhibition of prostaglandin E2 (PGE₂) receptor as a treatment option for endometriosis and other chronic gynecological diseases in reproductive age women. The present invention offers the advantages of: (i) decreased cell survival and increase in cell death/apoptosis pathways thereby inhibiting growth and survival of human endometriosis cells and inhibition of migration and invasion of endometriosis cells; (ii) decreased inflammation and disease burden and pain; and (iii) allowance for normal menstruation and sex life without any compromises in pregnancy in women.

One embodiment the present invention is a method of treating one or more chronic gynecological diseases in a subject comprising the steps of: (i) identifying the subject in need of treatment against the one or more chronic gynecological diseases and (ii) administering a pharmaceutical composition comprising a therapeutically effective amount of one or more selective inhibitors of prostaglandin E2 (PGE₂) receptors sufficient to treat the one or more chronic gynecological diseases. The one or more chronic gynecological diseases described in the invention are selected from endometriosis, dysmenorrhea, dyspareunia, non-cyclic pelvic and abdominal pain, subfertility, infertility and pelvic cancer. In one aspect the subject is a female subject in the age group of 12 to 50 years. Another aspect of the present invention further comprises the steps of: monitoring the progression of the one or more chronic gynecological diseases following the administration of the pharmaceutical composition and continuing, terminating or modifying the administration of the pharmaceutical composition based on the progression of the one or more chronic gynecological diseases, wherein the modification comprises an increase or a decrease in a dosage, a frequency or both of the pharmaceutical composition.

In a further aspect the one or more inhibitors at least partially and selectively inhibit PGE₂ receptors EP2 and EP4 selected from 6-Isopropoxy-9-oxoxanthene-2-carboxylic acid and (4Z)-7-[(rel-1S,2S,5R)-5-((1,1′-Biphenyl-4-yl)methoxy)-2-(4-morpholinyl)-3-oxocyclopentyl]-4-heptenoic acid and salts thereof. The inhibitors described in the present invention are administered at concentrations of 10 μM, 25 μM, 50 μM, 75 μM, 100 μM, 250 μM, 500 μM, and 1000 μM and are administered subcutaneously, intravenously, intraperitoneally, orally, intramuscularly, and intravaginally.

In yet another aspect the pharmaceutical composition for treating endometriosis prevents the growth, survival, migration and invasion of one or more endometriosis epithelial cells and stromal cells by modulating one or more G-protein coupled receptor mediated cell signaling pathways as determined by the activation at least one of the ERK1/2, AKT, NFκB or β-catenin signaling pathways, wherein the one or more cell signaling pathways comprise epidermal growth factor receptor (EGFR), nuclear factor kappa B (NFκB), and β-catenin/Wnt.

In a related embodiment the present invention discloses a pharmaceutical composition for treating one or more chronic gynecological diseases in a subject comprising: a therapeutically effective amount of one or more selective inhibitors of prostaglandin E2 (PGE₂) receptors sufficient to treat the one or more chronic gynecological diseases dissolved, dispersed, or suspended in an aqueous or a non-aqueous solvent and one or more optional excipients, fillers, diluents, extended or controlled release agents, bulking agents, antiadherents, binders, lubricants, preservatives or any combinations thereof. In one aspect the pharmaceutical composition is used in the treatment of one or more chronic gynecological diseases selected from endometriosis, dysmenorrhea, dyspareunia, non-cyclic pelvic and abdominal pain, subfertility, infertility and pelvic cancer. In another aspect the subject is a female subject in the age group of 12 to 50 years. In yet another aspect the one or more inhibitors at least partially and selectively inhibit PGE₂ receptors EP2 and EP4 selected from 6-Isopropoxy-9-oxoxanthene-2-carboxylic acid and (4Z)-7-[(rel-1S,2S,5R)-5-((1,1′-Biphenyl-4-yl)methoxy)-2-(4-morpholinyl)-3-oxocyclopentyl]-4-heptenoic acid and salts thereof. The one or more inhibitors are administered subcutaneously, intravenously, intraperitoneally, orally, intramuscularly and intravaginally at concentrations of 10 μM, 25 μM, 50 μM, 75 μM, 100 μM, 250 μM, 500 μM and 1000 μM.

In further aspects the pharmaceutical composition for treating endometriosis prevents the growth, survival, migration and invasion of one or more endometriosis epithelial cells and stromal cells, by the modulation of one or more G-protein coupled receptor mediated cell signaling pathways as determined by the activation at least one of the ERK1/2, AKT, NFκB or β-catenin signaling pathways. The one or more cell signaling pathways described in the present invention comprise epidermal growth factor receptor (EGFR), nuclear factor kappa B (NFκB), and β-catenin/Wnt.

Another embodiment of the present invention is a method of treating endometriosis in one or more female subjects in the age group of 12 to 50 years in a subject comprising the steps of: identifying the subject in need of treatment against endometriosis and administering a pharmaceutical composition comprising a therapeutically effective amount of one or more selective inhibitors of prostaglandin E2 (PGE₂) receptors sufficient to treat the endometriosis. The method described in the invention further comprises the steps of: monitoring the progression of the endometriosis following the administration of the pharmaceutical composition and continuing, terminating or modifying the administration of the pharmaceutical composition based on the progression of the endometriosis, wherein the modification comprises an increase or a decrease in a dosage, a frequency or both of the pharmaceutical composition. In a related aspect the one or more inhibitors at least partially and selectively inhibit PGE₂ receptors EP2 and EP4 and are selected from 6-Isopropoxy-9-oxoxanthene-2-carboxylic acid and (4Z)-7-[(rel-1S,2S,5R)-5-((1,1′-Biphenyl-4-yl)methoxy)-2-(4-morpholinyl)-3-oxocyclopentyl]-4-heptenoic acid and salts thereof. In further aspects the one or more inhibitors are administered at concentrations of 10 μM, 25 μM, 50 μM, 75 μM, 100 μM, 250 μM, 500 μM and 1000 μM and administered subcutaneously, intravenously, intraperitoneally, orally, intramuscularly, and intravaginally.

The pharmaceutical composition for treating endometriosis as described in the present invention prevents the growth, survival, migration and invasion of one or more endometriosis epithelial cells and stromal cells by the modulation one or more G-protein coupled receptor mediated cell signaling pathways as determined by the activation at least one of the ERK1/2, AKT, NFκB or β-catenin signaling pathways. The one or more cell signaling pathways described in the present invention comprise epidermal growth factor receptor (EGFR), nuclear factor kappa B (NFκB), and β-catenin/Wnt.

### BRIEF DESCRIPTION OF THE DRAWINGS

For a more complete understanding of the features and advantages of the present invention, reference is now made to the detailed description of the invention along with the accompanying figures and in which:

FIGS. 1A-1Q shows the immunohistochemical localization of PGE₂ receptors EP1, EP2, EP3 and EP4 proteins in ectopic and eutopic endometria in women during the proliferative phase of the menstrual cycle: (FIGS. 1A-1D): Ectopic endometrial, (FIGS. 1E-1H): Eutopic endometria from women with endometriosis, (FIGS. 1I-1L): Eutopic endometria from women without endometriosis, (FIGS. 1M-1P): Negative controls (control serum or IgG). GLE: Glandular epithelium; STR: Stroma and (FIG. 1Q): Densitometry of relative spatial expression of EP1, EP2, EP3 and EP4 receptors in GLE and STR, quantified using Image-Pro Plus. *-ectopic vs. eutopic endometria, (P<0.05) n=12. Relative spatial expression of EP2 and EP4 proteins in GLE and STR were higher in ectopic compared to eutopic endometria. Immunohistochemistry was performed using Vectastain Elite ABC kit and representative photomicrographs at 40× magnification are shown;

FIGS. 2A-2C show the histological characteristics of endometriosis-like lesions induced by immortalized human endometriosis epithelial and stromal cells in nude mice. Cross-section of multiple endometriosis-like lesions is shown at original magnifications of ×50 (FIG. 2A) and ×400 (FIGS. 2B and 2C); (FIG. 2A): Note the presence of well-developed and organized endometriosis glands (red arrow) and developing and organizing endometriosis glands (black arrow) in the submesothelial fatty tissues. SMFC=submesothelial fat cells; BV=blood vessels, (FIG. 2B): Well-developed and organized endometriosis gland with acini, and (FIG. 2C): Developing and organizing endometriosis glands with absence of acini. These endometriosis glands were lined with cuboidal or flattened glandular epithelial cells and surrounded by dense stromal cells. GLE=glandular epithelial cells; STR=stromal cells. Numerous blood vessels and capillaries were formed around the endometriosis glands. The blood vessels were lined with flattened endothelial cells and filled with red blood cells (FIG. 2B);

FIGS. 3A-3F show the immunohistochemical characterization of endometriosis-like lesions induced by immortalized human endometriosis epithelial and stromal cells in nude mice: (FIG. 3A): Expression of human-specific cytokeratin protein in endometriosis glandular epithelial cells, (FIG. 3B): Expression of human-specific vimentin protein in endometriosis stromal cells, (FIG. 3C): Expression of PCNA in endometriosis glandular epithelial and stromal cells, (FIG. 3D): Expression of MMP2 protein in endometriosis glandular epithelial and stromal cells, (FIG. 3E): Expression of ERα protein in endometriosis glandular epithelial and stromal cells, and (FIG. 3F) Negative control with serum or IgG. GLE=glandular epithelial; STR=stromal cells; SMFC=submesothelial fat cells;

FIGS. 4A-4F show the expression of: (FIG. 4B): COX-2, (FIG. 4C): EP2, and (FIG. 4D) EP4 proteins in glandular epithelium (GLE) and stroma (STR) of peritoneal endometriosis lesions induced by xenograft of mixed populations of immortalized human endometriotic epithelial cells 12Z and stromal cells 22B in nude mice. (FIG. 4A): Negative control. SMFC: submesothelial fat cells. Representative photomicrograph of (FIG. 4E) well organized endometriosis gland and (FIG. 4F) disorganized endometriosis gland in response to EP2-EP4-I were shown. Immunohistochemistry was performed by ABC kit;

FIG. 5A shows selective blockade of EP2 (AH6809) and EP4 (AH23848) inhibited growth of human endometriotic epithelial cells 12Z in the peritoneal cavity of xenograft recipient nude mice in temporal pattern. *Cont vs. EP2/EP4 inhibitors (EP-I) at 5, 10, or 25 mg/kg, P<0.05, n=4, (FIGS. 5B-5C): Representative nude mice showed xenograft of 12Z-GIN-GFP cells by whole animal bioimaging. (FIG. 5B) Control and (FIG. 5C) EP-I (25 mg/kg), (FIGS. 5D-5E): Disseminated 12Z-GIN-GFP cells in the peritoneal cavity of nude mice (representative xenograft) determined by zoom-stereo fluorescence dissection microscope, (FIG. 5D) Control and (FIG. 5E)-EP-I (25 mg/kg), (FIG. 5F): Number of 12Z-GFP cell clusters determined by zoom-stereo fluorescence dissection microscope, * Cont vs. EP-I (25 mg/kg), P<0.05, n=4, (FIG. 5G): Confocal microscopy analyses of disseminated and organized 12Z-GIN-GFP cells in the peritoneum after paraffin fixation, and (FIG. 5H): Fluorescence microscopy analyzes of 12Z-GIN-GFP cells before xenograft. Numerical data were analyzed by ANOVA and expressed as mean±SEM;

FIGS. 6A-6H show the effects of inhibition of EP1, EP2 and EP4 receptors on proliferation of human endometriotic epithelial and stromal cells: Dose response experiment for EP1, EP2 and EP4 inhibitors in endometriotic epithelial cells 12Z (FIG. 6A) and endometriotic stromal cells 22B (FIG. 6B). Pharmacological inhibition of EP2, EP4 or EP2/EP4 decreased proliferation of 12Z cells (FIG. 6C) and 22B cells (FIG. 6D), *: Control (CONT) vs. EP2-I, EP4-I or EP2-I/EP4-I; P<0.05. Pharmacological inhibition of EP2, EP4 or EP2/EP4 decreased viability of 12Z cells (FIG. 6E) and 22B cells (FIG. 6F),*: Control (CONT) vs. EP2-I, EP4-I or EP2-I/EP4-I; P<0.05. Knock-down of EP2, EP4, EP2/EP4 genes decreased proliferation of 12Z cells (FIG. 6G) and 22B cells (FIG. 6H), *: Control (CONT) vs. EP2⁻, EP4⁻ or EP2⁻/EP4⁻; P<0.05. Numerical values are expressed as the mean±SEM of three independent experiments;

FIGS. 7A-7N show the effects of inhibition of EP2/EP4 on regulation of cell cycle in human endometriotic epithelial and stromal cells. Inhibition of EP2/EP4 arrests progression of endometriotic epithelial cells 12Z through G1-S and G1-M checkpoints (FIG. 7A) and endometriotic stromal cells 22B through G2-M checkpoint, (FIG. 7B). Control versus EP2-I/EP4-I, *P<0.05. Western blot analyses on expression and regulation of CDK4, CDK6, CDK2, and CDK1 (FIGS. 7C-7F), cyclin D1, D2, D3, E2, A, and B1 (FIGS. 7G-7J), and CDK inhibitor p15, p16, p21, and p27 (FIGS. 7K-7N) proteins in 12Z and 22B cells. Control versus EP2-I/EP4-I, *P<0.05. Numerical values are expressed as the mean±SEM of three independent runs. CONT=control;

FIGS. 8A-8E show the selective inhibition EP2 and EP4 decreases proliferation of human endometriotic cells but not of eutopic endometrial cells: (FIG. 8A): Ectopic endometriotic epithelial cells 12Z. a: control versus PGE₂; b: control versus EP2/EP4-I; c: PGE₂ versus PGE₂+EP2/EP4-I; P<0.05, (FIG. 8B): Eutopic endometrial epithelial cells (HES) from endometriosis-free women, (FIG. 8C): Ectopic endometriotic stromal cells 22B. d: control versus PGE₂; e: control versus EP2/EP4-I; f: PGE2 versus PGE₂+EP2/EP4-I; P<0.05, (FIG. 8D): Eutopic endometrial stromal cells (HESC) from endometriosis-free women, and (FIG. 8E): PGE2 production by 12Z, HES, 22B, HESC at basal condition. g: 12Z versus HES; h: 22B versus HESC; P<0.05. Numerical values are expressed as the mean±SEM of three independent runs;

FIGS. 9A and 9B are schematic representations of the proposed EP2/EP4-mediated PGE₂ signaling on regulation of cell cycle in human endometriotic epithelial and stromal cells. (FIG. 9A) Inhibition of EP2/EP4 decreased proliferation of human endometriotic epithelial cells 12Z through [1] down-regulation of G1-specific cyclins D1, D2, D3, and CDK4 and CDK6; [2] down-regulation of G1-S transition-specific cyclin E2 and CDK2; [3] down-regulation of S-specific cyclin A and CDK2; [4] down-regulation of G2-specific cyclin A and CDK1; and [5] down-regulation of M-specific cyclin B1 and CDK1. Deregulated assembly between cell-specific CDKs and their cyclins (1 and 2) inhibits progression of cells through G1 and from G to S phases and thus leads to cell cycle arrest at G1-S checkpoint (6). Deregulated assembly between cell-specific CDKs and their cyclins (3, 4, and 5) inhibits progression of cells through G2 and M and results in cell cycle arrest at G2-M checkpoint (7). (FIG. 9B) Inhibition of EP2/EP4 decreased proliferation of human endometriotic stromal cells 22B through [1] down-regulation of S-specific cyclin A and CDK2; [2] down-regulation of G2-specific cyclin A and CDK1; and [3] down-regulation of M-specific cyclin B1 and CDK1. Deregulated assembly between cell-specific CDKs and their cyclins (1, 2, and 3) inhibits progression of cells through G2 and M and results in cell cycle arrest at G2-M checkpoint (4);

FIGS. 10A-10P represents the selective inhibition of EP2 and EP4 induces apoptosis of human endometriotic epithelial cells 12Z (Panel-1) and stromal cells 22B (Panel-2): (FIGS. 10A-10D): TUNEL assay based on flow cytometry, (FIGS. 10A and 10C): pharmacologic inhibition of EP1, EP2 and EP4, (FIGS. 10B and 10D): EP2 and EP4 siRNA, (FIGS. 10E-10H): Representative DNA histogram. Cells under M1 showed apoptotic cell population, (FIGS. 10I-10L): TUNEL assay based on fluorescence microscopy. Representative photomicrographs at 40× magnification are shown. Arrows show DNA fragments in TUNEL labeled cells. The cells were treated with EP inhibitors (EP-I) for EP1 (SC19220-100 μM), EP2 (AH6809-75 μM) and EP4 (AH23848-50 μM) for 24 h. *-control vs. EP-I or siRNA, P<0.05, n=3 Inhibition of EP2, EP4 or EP2/EP4 increases apoptosis in 12Z and 22B cells, and (FIGS. 10M-10P): Effects of EP2 or EP4 siRNA on expression of EP2 and EP4 proteins at 96 h post-siRNA. **-control vs. EP2 siRNA, ***-control vs. EP4 siRNA, P<0.05, n=3; EP2 and EP4 siRNA levels were 70-80%;

FIGS. 11A-11J shows the selective inhibition of EP2 and EP4 activates cytochrome C/caspase-3/PARP pathway in the human endometriotic epithelial cells 12Z (Panel-1) and stromal cells 22B (Panel-2): (FIGS. 11A and 11F): Western blot of cytochrome C, (FIGS. 11B and 11G): Western blot of caspase-3, (FIGS. 11C and 11H): Western blot of PARP, (FIGS. 11D, 11E, 11I, and 11J): Immunofluorescence analysis, (FIGS. 11D and 11I): cleaved caspase-3, and (FIGS. 11E and 11J): cleaved PARP. Arrows show cytosolic and nuclear localization of caspase-3 and PARP proteins, respectively, in cells undergoing apoptosis. Mitochondrial specific voltage dependent anion channel (VDAC) and cytosolic specific β-actin were measured as internal control proteins. *-control vs. EP2-I/EP4-I, P<0.05, n=3. The cells were treated with EP inhibitors (EP-I) for EP2 (AH6809-75 μM) and EP4 (AH23848-50 μM) for 24 h Inhibition of EP2 and EP4 facilitated release of cytochrome C (Cyt-c) from mitochondria (Mito) into cytosol (Cyto) and activated/cleaved caspase-3 and PARP proteins in 12Z and 22B cells;

FIGS. 12A-12H shows the selective inhibition of EP2 and EP4 augments interactions between antiapoptotic and proapoptotic proteins in the human endometriotic epithelial cells 12Z (Panel-1) and stromal cells 22B (Panel-2): (FIGS. 12A and 12E): Western blot of Bcl-2 and Bcl-XL, (FIGS. 12B and 12F): Western blot of Bax and Bad, (FIGS. 12C, 12D, 12G, and 12H): Immunoprecipitation/Western blot, (FIGS. 12C and 12G): Interaction between Bax and Bcl2 or Bcl-XL, and (FIGS. 12D and 12H): Interaction between Bad and Bcl2 or Bcl-XL. β-actin was measured as an internal control. *-control vs. EP2-I/EP4-I, P<0.05, n=3. The cells were treated with EP inhibitors (EP-I) for EP2 (AH6809-75 μM) and EP4 (AH23848-50 μM) for 24 h. Inhibition of EP2 and EP4 decreased expression of Bcl-2 and Bcl-XL, increased expression of Bax, dephosphorylated Bad at serine 112 and 136 sites, and increased interactions between Bax and Bcl-2; Bax and Bcl-XL; Bad and Bcl-2; and Bad and Bcl-XL proteins in 12Z and 22B cells;

FIG. 13A is a schematic showing that the EP2/EP4-mediated PGE₂ signaling leads to cell survival. PGE₂ could transactivate ERK1/2, AKT, NFκB, and β-catenin pathways through EP2 and EP4 receptors. Activation of these cell survival pathways phosphorylates Bad protein at serine 112 and 136, sequestrates Bad and Bax proteins in the cytosol with 14-3-3 proteins, and prevents translocation of Bad and Bax from the cytosol to the mitochondria. This prevents interactions between Bax/Bad and Bcl-2/Bcl-XL, and thereby promotes survival of human endometriotic cells;

FIG. 13B is a schematic showing that the inhibition of EP2/EP4-mediated PGE₂ signaling leads to cell apoptosis. Selective inhibition of EP2 and EP4 impairs ERK1/2, AKT, NFκB, and β-catenin pathways. Inhibition of these cell survival pathways results in dephosphorylation of Bad protein at serine 112 and 136, dissociation of Bad and Bax from 14-3-3 proteins, permits translocation of Bad and Bax proteins from the cytosol to the mitochondria, and thereby augments interaction between Bax/Bad and Bcl-2/Bcl-XL proteins. These sequential interactions result in release of cytochrome-C from the mitochondria into the cytosol and activation of caspase-3 and PARP enzymes and eventually culminate in apoptosis of human endometriotic cells;

FIGS. 14A-14G shows the selective blockade of EP2 and EP4 inhibited invasion of human endometriotic: (FIG. 14A): Epithelial cells 12Z and (FIG. 14B) Stromal cells 22B, (FIG. 14C): Western blot and (FIG. 14D): Densitometry analyses of MMPs and TIMPs proteins, Zymography of (FIG. 14E) MMP2 and (FIG. 14F) MMP9 activity, and (FIG. 14G): Western blot analyses of EMMPRIN and MT1-MMP proteins. * control vs. EP2-I/EP4-I. Invasion potential of 12Z and 22B cells (** 12Z vs. 22B, P<0.05). Temporal invasion of 12Z cells (a: 12 vs. 24 h, P<0.05) and 22B cells (b: 12 vs. 24 h, P<0.05). The 12Z and 22B cells were cultured and treated with EP inhibitors (EP-I) for EP2 (AH6809-75 μM) and EP4 (AH23848-50 μM) for 24 h as described in Preliminary Study 2, n=3. Numerical data were analyzed by ANOVA and expressed as mean±SEM;

FIGS. 15A-15C are schematic representations of molecular mechanisms through which selective inhibition of EP2 and EP4 inhibits invasion of human endometriotic cells: (FIG. 15A): EP2/EP4-mediated signaling in invasion of human endometriotic epithelial and stromal cells. (FIG. 15B): EP2/EP4-mediated degradation of ECM and invasion of human endometriotic epithelial and stromal cells, and (FIG. 15C): Selective inhibition of EP2 and EP4 inhibits invasion of human endometriotic epithelial and stromal cells;

FIGS. 16A-16C shows that PGE₂ transactivates EGFR in the human endometriotic epithelial cells 12Z and stromal cells 22B: (FIG. 16A): Western blot of EGFR, ERK1/2 and AKT, (FIG. 16B): Western blot of c-Src and β-arrestin 1, and (FIG. 16C): Immunoprecipitation/Western blot showed the interactions among EP2, EP4, EGFR, c-Src and β-arrestin (β-arr 1) 1. β-actin was measured as an internal control. *-control vs. EP2-I/EP4-I, P<0.05, n=3. The cells were treated with EP inhibitors (EP-I) for EP2 (AH6809-75 μM) and EP4 (AH23848-50 μM) for 24 h. Inhibition of EP2 and EP4 affected phosphorylation of EGFR, ERK1/2, AKT, c-Src, and β-arrestin 1 in a cell-specific manner, and decreased interactions among EP2/EP4, c-Src, β-arrestin 1 and EGFR proteins in 12Z and 22B cells;

FIGS. 17A-17C shows that PGE₂ transactivates NFκB pathways in the human endometriotic epithelial cells 12Z and stromal cells 22B: (FIG. 17A): Western blot of IkB, p105/p50, p65, (FIG. 17B): Western blot of TNFαR1 and IL1βR1, and (FIG. 17C): Immunoprecipitation/Western blot showed the interactions among TNFαR1, IL1βR1, c-Src and β-arrestin 1 (β-arr 1). β-actin was measured as an internal control. *-control vs. EP2-I/EP4-I, P<0.05, n=3. The cells were treated with EP inhibitors (EP-I) for EP2 (AH6809-75 μM) and EP4 (AH23848-50 μM) for 24 h Inhibition of EP2 and EP4 dephosphorylated IκB_{α}, and p65, decreased expression of p105, p50, TNFαR1, and IL1βR1, and decreased interaction between c-Src/β-arrestin 1 complex and TNFαR1 or IL1βR1 proteins in 12Z and 22B cells;

FIGS. 18A-18D shows that PGE₂ transactivates β-catenin pathways in the human endometriotic epithelial cells 12Z and stromal cells 22B: (FIG. 18A): Western blot of β-catenin (β-cate), TCF-1, TCF-4 and LEF-1, (FIG. 18B): Western blot of p-GSKα/β, t-GSKα/βaxin, and Gs. β-actin was measured as an internal control, and (FIGS. 18C and 18D): Immunoprecipitation/Western blot showed the interactions among axin, Gs, and β-catenin, and interactions among GSK3β, p-ERK1/2, p-AKT, and β-catenin. *-control vs. EP2-I/EP4-I, P<0.05, n=3. The cells were treated with EP inhibitors (EP-I) for EP2 (AH6809-75 μM) and EP4 (AH23848-50 μM) for 24 h. Inhibition of EP2 and EP4 decreased active β-catenin, TCF-1 and TCF-4, dephosphorylated GSK3β, and affects interactions between axin and Gs; GSK3β and AKT; and GSK3β and ERK1/2 proteins in a cell-specific manner in 12Z and 22B cells;

FIG. 19 shows that PGE₂ regulates expression of multiple transcriptional factors in human endometriotic epithelial cells 12Z and stromal cells 22B Inhibition of EP2 and EP4 affects expression/activation of c-fos, c-Jun, c-myc, CRBE, Sp1, and EGR-1 proteins in a cell-specific manner in 12Z and 22B cells. The cells were treated with EP inhibitors (EP-I) for EP2 (AH6809-75 μM) and EP4 (AH23848-50 μM) for 24 h. β-actin was measured as an internal control. *-control vs. EP2-I/EP4-I, P<0.05, n=3;

FIGS. 20A-20T shows the expression of PGE₂ signaling components in ectopic and eutopic endometria during proliferative phase of the menstrual cycle in women: (FIGS. 20A and 20B): IgG or Serum, (FIGS. 20C and 20D): Bcl-2, (FIGS. 20E and 20F): Bcl-XL, (FIGS. 20G and 20H): p-Bad112, (FIGS. 20I and 20J): p-Bad136, (FIGS. 20K and 20L): p-EGFR. (FIGS. 20M and 20N) p-ERK1/2, (FIGS. 20O and 20P): p-AKT, (FIGS. 20Q and 20R): p-IκB, (FIGS. 20S and 20T): Active β-catenin, and (FIG. 20U): Densitometry of relative spatial expression of these proteins in glandular epithelium (GLE) and stroma (STR). *-ectopic vs. eutopic endometria, P<0.05, n=12. Relative spatial expression of PGE₂ signaling proteins in glandular epithelium and stroma were higher in ectopic compared to eutopic endometria. Immunohistochemistry was performed using Vectastain Elite ABC kit and representative photomicrographs at 40× magnification were shown; and

FIG. 21 is a schematic showing the PGE₂-EP2/EP4 signaling network in the human endometriotic cells: PGE₂ binds with EP2 and EP4 receptors. Upon activation, these receptors are coupled to Gs proteins and activate three major signaling pathways: (i) EP2/EP4 interacts with c-Src kinase and β-arrestin 1 protein complex and transactivates EGFR intracellularly which in turn activates ERK1/2 and AKT pathways; (ii) EP2/EP4 interacts with c-Src kinase and β-arrestin 1 and transactivates TNFαR1 and IL1β1R intracellularly which consecutively activates NFκB pathways and (iii) EP2/EP4 activates β-catenin pathways intracellularly by disestablishing Gs and axin complex directly and inhibiting GSK3β through AKT and ERK1/2 pathways. Transactivation of these three linear-cell signaling pathways activates specific transcriptional/DNA complexes.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein the term “estrogens” refers to the substances tending to promote estrus and stimulate the development of female secondary sex characteristics. This term comprises natural, semisynthetic and synthetic estrogens, both steroidal and nonsteroidal, such as estrone, diethylstilbestrol, estriol, estradiol and ethinyl estradiol. In this specification the term “fatty acids” refers to the carboxylic acids which are components of natural fats, such as oleic, linoleic, linolenic, stearic, palmitic, palmitoleic, and arachidonic acids. The term “mitogen” as used in the specification defines those substances that stimulate the division of cells which would otherwise (i.e., without the influence of this substance) not divide.

The term “gynecological diseases” as used herein comprises but are not limited to diseases or disorders that affect the organs in a woman's abdominal and pelvic areas.

The term “prostaglandin” refers to those cyclopentane-containing carboxylic acids derived from mammalian tissues which are structural derivatives of prostanoic acid. The term “epithelial cell” is used herein to refer to the epithelium, i.e., the covering of internal and external surfaces of the body, including the lining of vessels and the small cavities. These cells are present in the colon, breast, ovarian, prostate, kidneys, etc. As used herein, the term “stromal cells” includes: (1) human allogenic or autologous stromal cells, or non-human stromal cells, (2) human or non-human stromal cell lines which need not be hematopoietic, and (3) human or non-human virally infected cell lines, such as immortalized embryonic fibroblasts which are effective to provide “feeder layers” for stem cell populations.

As used herein the term, “in vitro” refers to an artificial environment and to processes or reactions that occur within an artificial environment. In vitro environments can comprise of, but are not limited to, test tubes and cell cultures. The term “in vivo” refers to the natural environment or to being inside the body (e.g., an animal or a cell) and to processes or reactions that occur within a natural environment.

As used herein, the term “receptor” includes, for example, molecules that reside on the surface of cells and mediate activation of the cells by activating ligands, but also is used generically to mean any molecule that binds specifically to a counterpart. One member of a specific binding pair would arbitrarily be called a “receptor” and the other a “ligand”. No particular physiological function need be associated with this specific binding. Thus, for example, a “receptor” might include antibodies, immunologically reactive portions of antibodies, molecules that are designed to complement other molecules, and so forth.

The term “gene” is used to refer to a functional protein, polypeptide or peptide-encoding unit. As will be understood by those in the art, this functional term includes genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

As used herein, the term “treatment” refers to the treatment of the conditions mentioned herein, particularly in a patient who demonstrates symptoms of the disease or disorder. As used herein, the term “treatment” or “treating” refers to any administration of a compound of the present invention and includes (i) inhibiting the disease in an animal that is experiencing or displaying the pathology or symptomatology of the diseased (i.e., arresting further development of the pathology and/or symptomatology) or (ii) ameliorating the disease in an animal that is experiencing or displaying the pathology or symptomatology of the diseased (i.e., reversing the pathology and/or symptomatology). The term “controlling” includes preventing treating, eradicating, ameliorating or otherwise reducing the severity of the condition being controlled.

The terms “effective amount” or “therapeutically effective amount” described herein means the amount of the subject compound that will elicit the biological or medical response of a tissue, system, animal or human that is being sought by the researcher, veterinarian, medical doctor or other clinician.

The terms “administration of” or “administering a” compound as used herein should be understood to mean providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

Endometriosis is a benign chronic gynecological disease of reproductive age women characterized by the presence of functional endometrial tissues (glandular epithelium and stroma) outside the uterine cavity. Endometriosis affects up to 10-20% of reproductive-age women and is associated with dysmenorrhea, dyspareunia, non-cyclic pelvic and abdominal pain, subfertility, infertility, and increased pelvic cancer susceptibility. The most widely accepted hypothesis is that viable endometrial tissue fragments are refluxed through the fallopian tubes into the pelvic cavity during retrograde menstruation (3).

Endometriosis is considered as an estrogen (E)-responsive (4) and P-unresponsive disease (6). Current treatment modalities targeted to decrease ovarian E production through the use of oral contraceptives (OC), aromatase inhibitors, androgenic agents, and GnRH analogues (1-2, 4) compromise menstruation, pregnancy, reproductive health of women, and fail to prevent reoccurrence of disease. Nevertheless, the recurrence rate is up to 50%-60% after cessation of hormonal therapy within 1 year (7). There is a critical need to identify new specific signaling modules for non-estrogen targeted therapies for endometriosis. Prostaglandin E2 (PGE₂) is a mitogen that plays central role in pathophysiology and pathogenesis of several diseases in human. PGE₂ effects are primarily mediated through G-protein coupled membrane receptors designated EP that includes EP1, EP2, EP3 and EP4. The concentrations of PGE₂ in peritoneal fluid are higher in women suffering from endometriosis compared to disease-free women. In endometriosis, much of the pain is due to the high levels of PGE₂ and associated inflammation. Inhibition of PGE₂ production by COX-2 inhibitors prevents establishment of endometriosis, decreases the size and number of endometriosis lesions, and prevents neoangiogenesis in endometriosis implants in different animal models for study of endometriosis. However, very limited published information is available in women to confirm or evaluate these observations. A recent placebo controlled double blind study reported that selective COX-2 inhibitor rofecoxib at minimal dosage (25 mg/d for 6 months) effectively suppressed the pelvic pain symptoms associated to endometriosis in women (16). Use of the GnRH agonist, leuprolide acetate (LA) at 3.75 mg, for 1 month decreased expression of COX-2 along with p450 aromatase in eutopic endometria of patients with endometriosis (58). In vitro studies indicated that the COX-2 inhibitor celecoxib prevented growth and survival of primary cultured eutopic endometrial epithelial cells from patients with endometriosis (23). The present inventors recently found that selective inhibition of COX-2 using NS-398 prevents survival, migration, and invasion of human immortalized endometriotic epithelial cells 12Z and stromal cells 22B, which is associated with decreased PGE₂ production (11). Collectively, these studies strongly indicate a prerequisite for COX-2/PGE₂ in the pathogenesis of endometriosis and inhibition of COX-2/PGE₂ could emerge as a potential therapy for this disease in humans.

Unfortunately, therapeutic use of COX-2 inhibitors has been suspended because of undesirable cardiovascular side effects which might be due to inhibition of other PGs such as PGI2 and TxA2 but not due to inhibition of PGE₂. Thus, there exists an imperative need to discover alternative targets down-stream of COX-2 to inhibit the action of PGE₂ at receptor levels. Previous studies by the present inventors have indicated that EP2 and EP4 receptors are abundantly expressed in endometriosis tissues (ectopic endometrium) compared to normal endometrial tissues (eutopic endometrium) in women. Results from in vitro studies, using immortalized human endometriosis cells as a model system, indicated that cells treated with inhibitors of EP2 (AH6809-75 μM) and EP4 (AH23848-50 μM) receptors prevented growth, survival, migration and invasion of endometriosis epithelial and stromal cells. These effects were achieved through modulating multiple cell signaling pathways such as epidermal growth factor receptor (EGFR), nuclear factor kabba B (NFkB) and β-catenin1/Wnt. The present invention discloses the combined selective inhibition of PGE₂ receptors EP2 and EP4, and its application as a potential therapy for treatment of endometriosis in women.

The inventors have previously reported that selective inhibition of cyclooxygenase-2 prevented survival, migration and invasion of human endometriotic epithelial and stromal cells which was due to decreased PGE₂ production. In the present invention mechanisms through which prostaglandin E2 (PGE₂) promoted survival of human endometriotic cells were determined. One of the key embodiments of the present invention describes selective inhibition of EP2 and EP4. The present invention further provides an important molecular framework for further evaluation of selective inhibition of prostaglandin receptors as a potential therapy, including non-estrogen targets, to expand the spectrum of currently available treatment options for endometriosis in women. The novel findings of the present invention for treating endometriosis can further be extended to be effective against other common gynecological problems in women.

Combined selective inhibition of PGE₂ receptors EP2 and EP4: (i) will decrease cell survival and increase cell death/apoptosis pathways thereby inhibits growth and survival of human endometriosis cells and inhibits migration and invasion of endometriosis cells, (ii) will decrease the inflammation, disease burden, and pain, (iii) will allow normal menstruation and sex life, and (iv) will not compromise pregnancy in women.

Some key results of the present invention indicate that: (i) EP2 and EP4 receptors induces apoptosis of endometriotic epithelial cells 12Z and stromal cells 22B cells through suppression of cell survival pathways EGFQ3 receptor, ERK1/ERK2, AKT, natural killer (NF) kB, and β-catenin, (ii) PGE2 promotes survival of human endometriotic cells through EP2 and EP4 receptors by activating ERK1/2, AKT, NFαB, and β-catenin signaling pathways, (iii) selective inhibition of EP2 and EP4 suppresses these cell survival pathways and augments interactions between proapoptotic proteins (Bax and Bad) and antiapoptotic proteins (Bcl-2/Bcl-XL), facilitates the release of cytochrome C and thus activates caspase-3/PARP-mediated intrinsic apoptotic pathways, (iv) these PGE2 signaling components are more abundantly expressed in ectopic endometriosis tissues compared to eutopic endometrial tissues during the menstrual cycle in women, and (v) inhibition of PGE2 production and action prevents proliferation of many cell types through cell cycle arrest at G1-S or G2-M checkpoints (59-69).

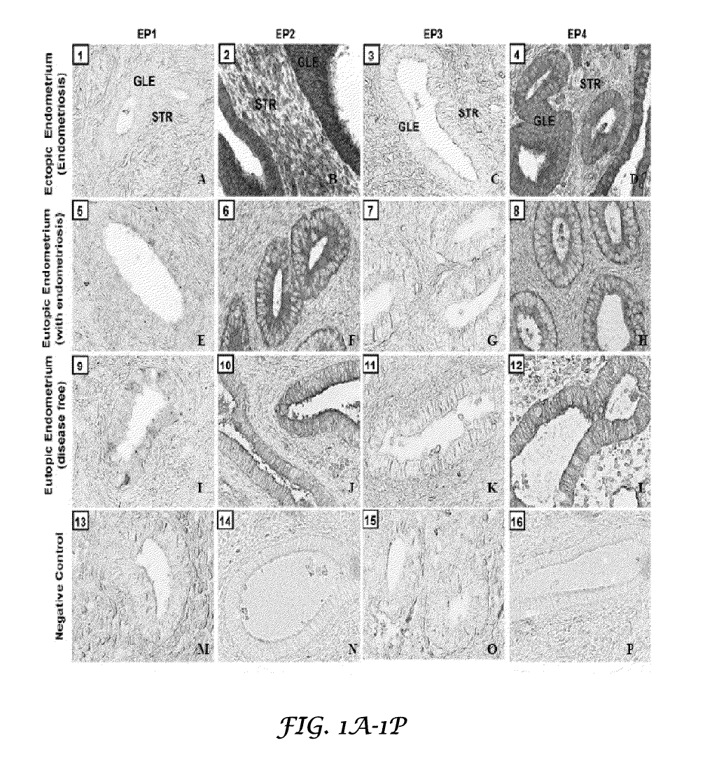

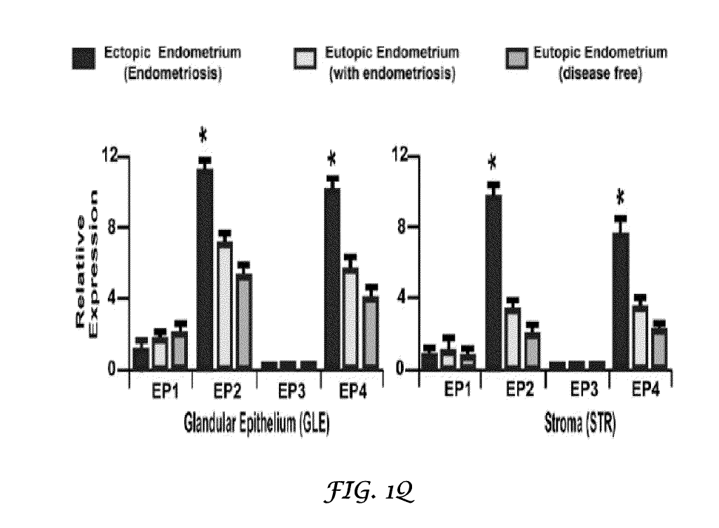

Expression of PGE₂ receptors in human endometriosis tissues: In deciphering PGE₂ signaling in pathogenesis of endometriosis, the inventors first determined relative expression of EP receptors in endometriosis and compared with endometrial tissues in women. Results (FIGS. 1A-1Q) indicated that EP2 and EP4 proteins were abundantly expressed (P<0.05), EP1 protein was expressed at very low levels (P<0.05) and EP3 protein was undetectable in endometriosis tissues (ectopic endometrium) and endometrial tissues (eutopic endometrium) from women with or without endometriosis during the proliferative phase of the menstrual cycle. Interestingly, relative spatial expression of EP2 and EP4 proteins in glandular epithelium and stroma were higher (P<0.05) in ectopic compared to eutopic endometria; however, this increase was not significant between eutopic endometria from women with and without endometriosis. These results suggest that EP2 and EP4 might be the major PGE₂ receptors that mediate PGE₂ signaling in the pathogenesis of endometriosis in women.

To better understand the complexity and uncertain etiology of this disease, the present inventors conducted a study using well-characterized human immortalized endometriosis epithelial (12Z) and stromal (22B) cells (11, 39, 82-85) to induce peritoneal endometriosis in nude mice. The main objectives of the study were [1] to determine the ability of a mixed population of human immortalized endometriosis epithelial and stromal cells to induce peritoneal endometriosis in nude mice, [2] to characterize these induced endometriosis-like lesions in nude mice by determining expression of proteins associated with cell proliferation and invasion, estrogen signaling, and prostaglandin biosynthesis and signaling; and [3] to compare the histomorphology of these induced peritoneal endometriosis like lesions in nude mice with spontaneous endometriosis lesions in women.

Seven ovariectomized nude mice were taken for the study and minimally invasive procedures were performed to administer estrogen pellets and transplant immortalized human endometriosis epithelial and stromal cells into the nude mice. The main outcome measured was the induction of peritoneal endometriosis-like lesions the in the nude mice and the characterization and comparison with spontaneous peritoneal endometriosis in women.

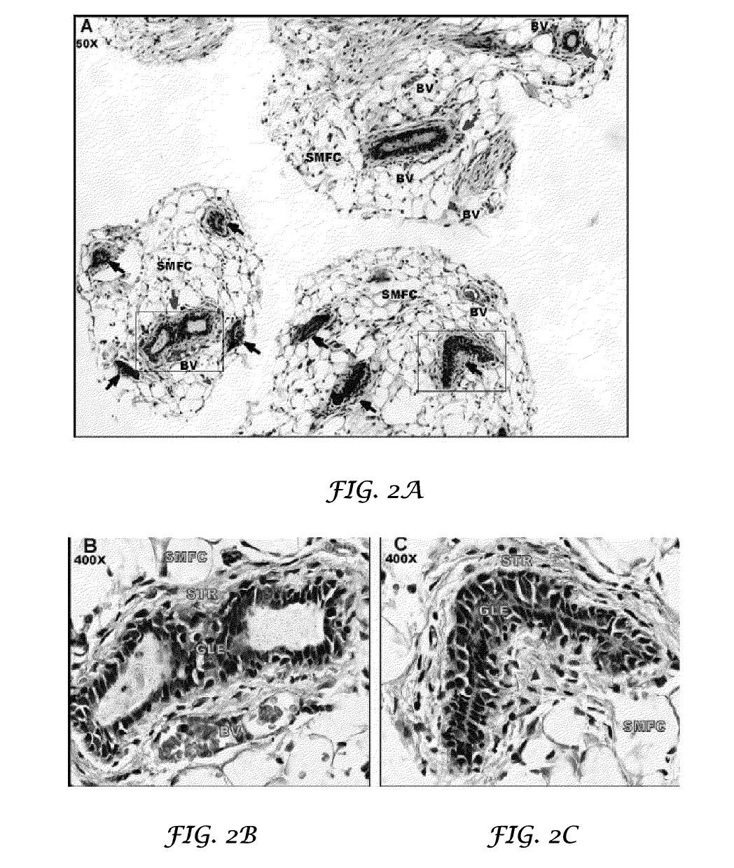

The results indicated that successful peritoneal endometriosis was induced with use of a mixed population of human immortalized endometriosis epithelial and stromal cells in nude mice. All seven nude mice transplanted with human immortalized endometriosis cells survived after the procedure and developed peritoneal endometriosis. The survival rate of nude mice and induction of peritoneal endometriosis was 100%. Gross visual examination of the peritoneum of the recipient nude mice revealed the presence of endometriosis-like lesions. The induced endometriosis-like lesions consisted of endometriosis glands lined with cylindrical and flattened epithelial cells and surrounded by dense stromal cells in the peritoneal submesothelial fatty tissues at variable sizes and organizations (FIGS. 2A-2C). Histomorphologic analyses demonstrated that endometriosis glands were present at different developmental and organization (formation of acini) states.

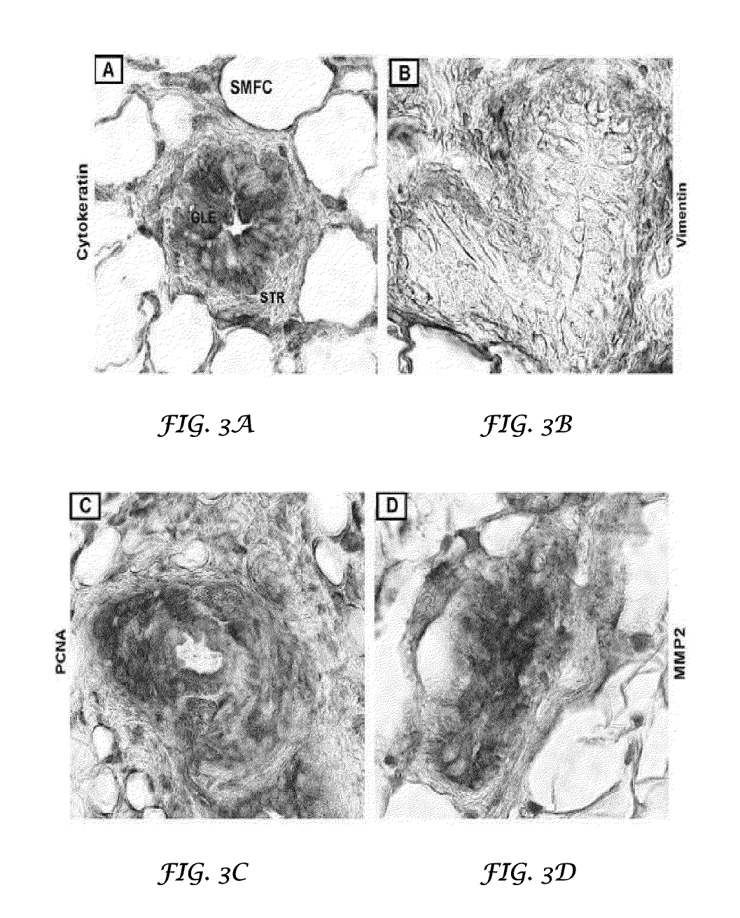

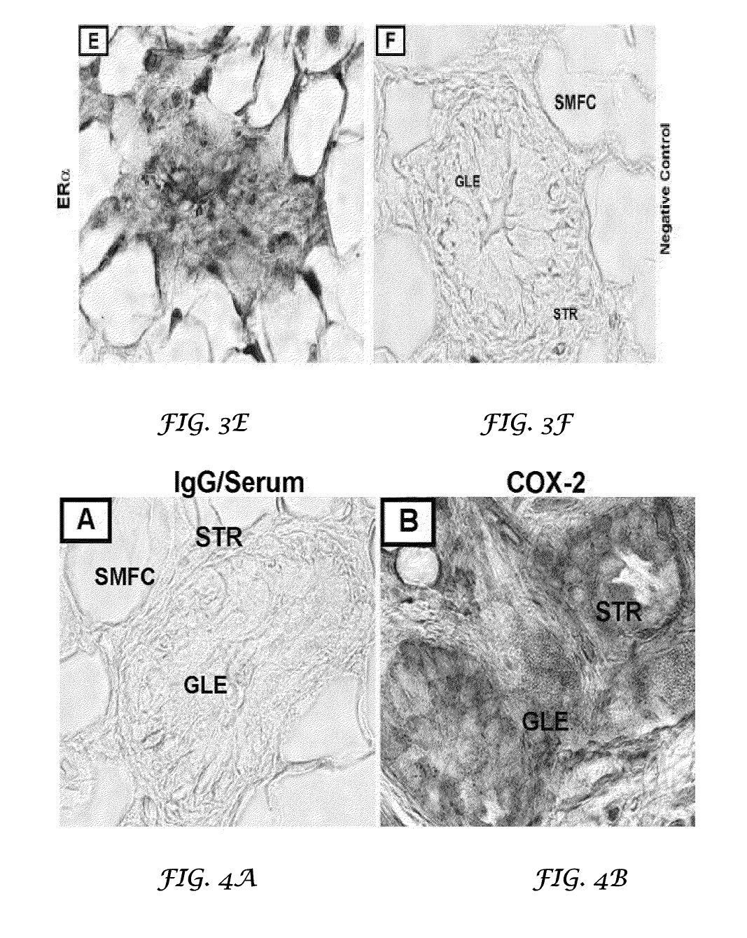

The inventors further characterized the lesions by determining the source of the cells present in the endometriosis-like lesions, by examining for the presence of Cytokeratin a specific epithelial marker protein and vimentin a specific stromal and fibroblast cell marker protein (86) by using human-specific cytokeratin 8-18 and vimentin V9 antibodies. Glandular epithelial cells were intensely stained for cytokeratin (FIG. 3A), and surrounding stromal cells were strongly stained for vimentin (FIG. 3B). The proliferation and invasion potential of endometriosis cells present in the induced peritoneal endometriosis-like lesions in nude mice was also determined. Proliferating cell nuclear antigen and MMPs are invariably considered as markers to estimate proliferation (87, 88) and invasion potential (89-91), respectively. Results indicated that PCNA (FIG. 3C) and MMP2 (FIG. 3D) proteins were expressed abundantly in endometriosis glandular epithelial and stromal cells. ERα protein is expressed in the induced peritoneal endometriosis-like lesions in nude mice. Results indicated that ERα protein (FIG. 3E) was expressed abundantly in both glandular epithelial and stromal cells of the induced peritoneal endometriosis-like lesions in nude mice that mediate estrogen action in vivo. FIG. 3F represented the negative control.

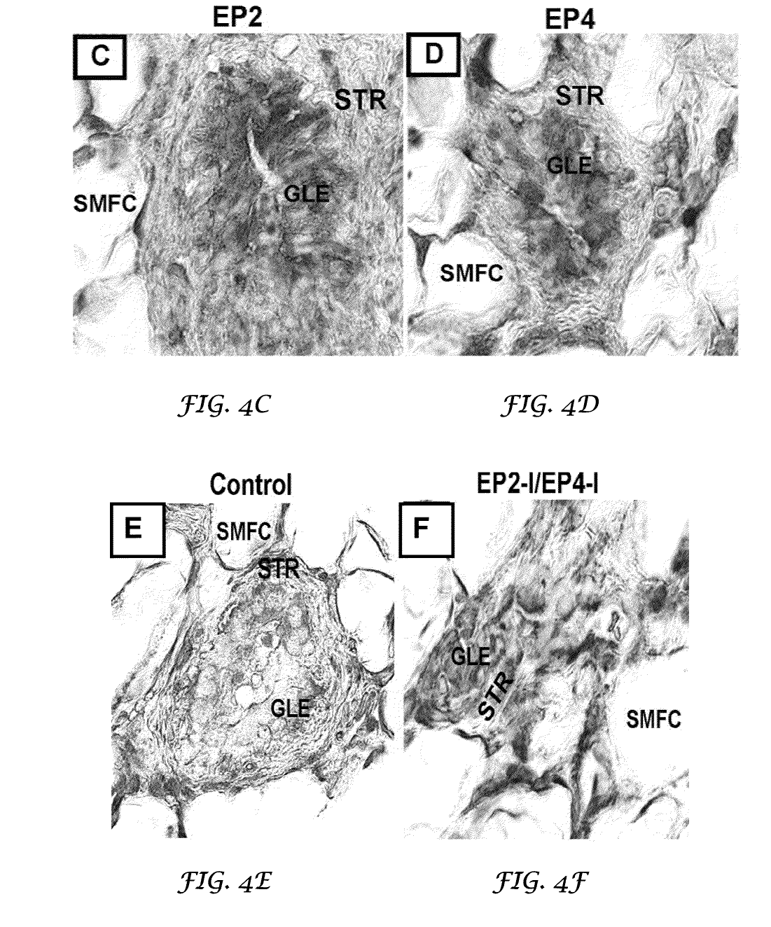

The inventors then evaluated the therapeutic efficacy of selective inhibition of EP2 and EP4 on survival and growth of endometriosis in vivo using a xenograft model. Endometriotic epithelial cells 12Z and stromal cells 22B were cultured and xenografted as described previously by the inventors (92). In order to simulate clinical condition the 12Z and 22B cells were allowed to grow for minimum of 14 days to establish endometriosis. The day of xenograft is considered as day 0. On day 15 post-xenograft the xenografted nude mice were divided into four groups based on 2×2 factorial design and received one of the following treatments subcutaneously from day 15-28 post-xenograft. Group-1 mice (n=4) were treated with vehicle (20% DMSO in 300 μl) and served as vehicle control. Group-2 (n=4), Group-3 (n=4), and Group-4 (n=4) mice were treated with inhibitors for EP2 (AH6809) and EP4 (AH23848) (Sigma-Aldrich) each at 5, 10, or 25 mg/kg body weight, respectively in a total volume of 300 μl in 20% DMSO. After 28 days of xenograft, all the xenografted mice were sacrificed, number and size of endometriosis lesions were determined, serial sections of peritoneum were cut, and organization of endometriosis glands were determined as described previously (92). The results are shown in FIGS. 4A-4D.

The results indicated that xenograft of a mixed population of 12Z and 22B cells into the peritoneal cavity of nude mice were able to proliferate, adhere, invade, reorganize, and establish peritoneal endometriosis-like lesions, and further COX-2, EP2 and EP4 proteins were abundantly expressed in these induced endometriosis-like lesions (92). EP2/EP4 inhibitors at 5 mg had no effect, 10 mg partially decreased the disease, and 25 mg inhibited (P<0.05) number and size of lesions and increased number of disorganized glands within 14 days of treatment. General health of xenograft recipient nude mice was not affected by any of the dose of EP2 and EP4 inhibitors. Thus, it suggests that subcutaneous injections of EP2/EP4 inhibitors at 25 mg/kg per day for 14 days were not toxic to the xenografted nude mice. These results together indicate that xenograft of mixed population of human endometriotic epithelial cells 12Z and stromal cells 22B are able to induce peritoneal endometriosis characterized by well organized glands, and selective inhibition of EP2 and EP4 perturb organization of endometriosis glands and survival of disease. Therefore, 12Z and 22B cells are ideal model systems to study molecular aspects of PGE₂ signaling in the pathogenesis of endometriosis in women.

Data from the characterization and the morphological analysis of the induced lesions is presented in Table 1. Table 2, presents the results from the inhibition studies described hereinabove.

TABLE 1
Gross morphologic analysis of endometriosis-like lesions induced by mixed
population of immortalized human endometriosis epithelial and stromal cells in nude mice.

| | | | | | | | | Size of | | |
|---|---|---|---|---|---|---|---|---|---|---|
| | | | | | | | | Endometriotic | | |
| | No. of Animals | Number of Endometriotic Lesions | | | | | | Lesions | | |
| Total No. | with | Total Lesions | | Single Lesions | | Multiple Lesions | | (cm) | | Color |
| of Animals | Endometriotic | Total | Mean ± | Total | Mean ± | Total | Mean ± | | Mean ± | of Endometriotic |
| Used | Lesions | No. | SE | No. | SE | No. | SE | Range | SE | Lesions |
| 7 | 7 | 45 | 6.43 ± 0.30 | 31 | 4.23 ± 0.56 | 14 | 2.01 ± 0.31 | 0.30-0.70 | 0.49 ± 0.02 | Pinkish Red |
| | (100%) | | | (68.89%) | | (31.11%) | | | | 28/45 (62.22%) |
| | | | | | | | | | | Whitish |
| | | | | | | | | | | 17/45 (37.78%) |

TABLE 2
Xenografted nude mice treated with inhibitors of EP2 (AH6809,
25 mg/kg) and EP4 (AH23848, 25 mg/kg) for 2 weeks.

| Parameters | Control | EP2-I/EP4-I |
|---|---|---|
| Number of lesions | 5.25 ± 0.25 | 2.75 ± 0.01 |
| Size of lesions | 0.43 ± 0.01 | 0.20 ± 0.02 |
| Number of organized gland per lesions | 3.62 ± 0.50 | 1.24 ± 0.20 |
| Number of disorganized gland per lesions | 0.05 ± 0.02 | 2.45 ± 0.15 |

* Control vs. EP2-I/EP4-I, P < 0.05, n = 4.
Numerical data analyzed by ANOVA and expressed as mean ± SEM.

A representative photomicrograph of a well organized endometriosis gland is shown in FIG. 4E, whereas FIG. 4F represents a disorganized endometriosis gland in response to EP2-EP4-I. Data from the histologic analysis of endometriosis-like lesions induced by mixed population of immortalized human endometriosis epithelial and stromal cells in nude mice are shown in Tables 3 and 4.

TABLE 3
Number of endometriotic glands in single and multiple lesions.
Number of Endometriotic Glands

| | No. of Glands in Single Lesions | | | No. of Glands in Multiple Lesions | | |
|---|---|---|---|---|---|---|
| Total | | No. of | No. of | | No. of | No. of |
| No. | Total | Glands/Lesion | Glands/Lesion | Total | Glands/Lesion | Glands/Lesion |
| Glands | No. | (Range) | (Mean ± SE) | No. | (Range) | (Mean ± SE) |
| 182 | 84 | 1-3 | 2.33 ± 0.24 | 98 | 4-8 | 6.53 ± 0.47* |
| | (46.15%) | | | (53.85%) | | |

TABLE 4
Characteristics of endometriotic glands.

| | Characteristics of Endometriotic Glands | | | |
|---|---|---|---|---|
| | No. of well | | | |
| | developed and | | No. of developing and | |
| | organized Glands | | unorganized Glands | |
| | Single | Multiple | Single | Multiple |
| | Lesions | Lesions | Lesions | Lesions |
| Total No of | 57/84 | 51/98 | 27/84 | 47/98 |
| Glands | | | | |
| Percentage (%) | 67.85 | 52.05 | 32.15 | 47.95 |
| Range | 1-3 | 2-5 | 0-2 | 2-5 |
| Mean ± SE | 1.58 ± 0.26 | 3.4 ± 0.35 | 0.60 ± 0.23 | 3.13 ± 0.40* |

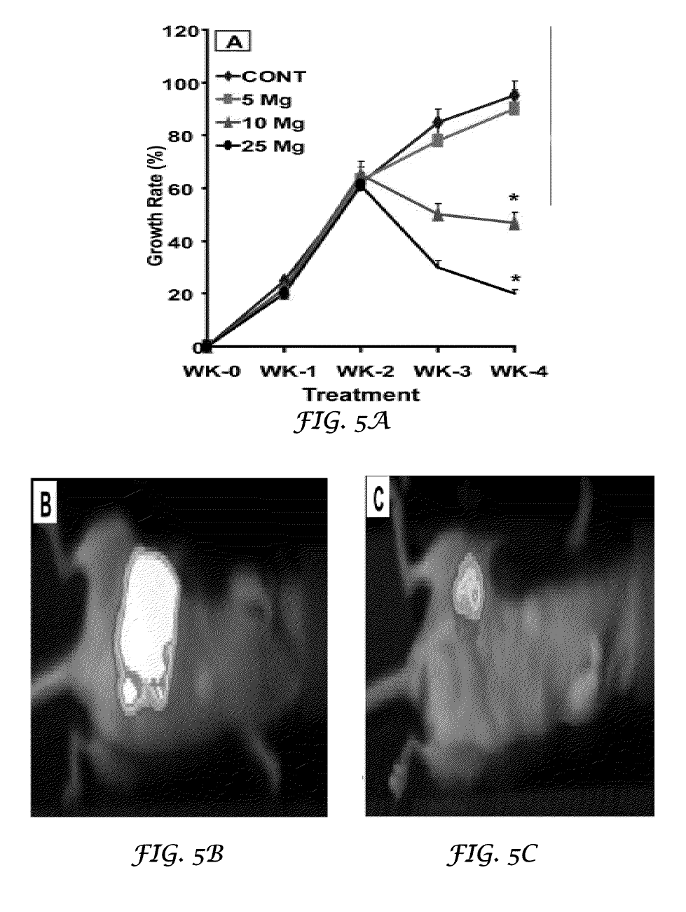

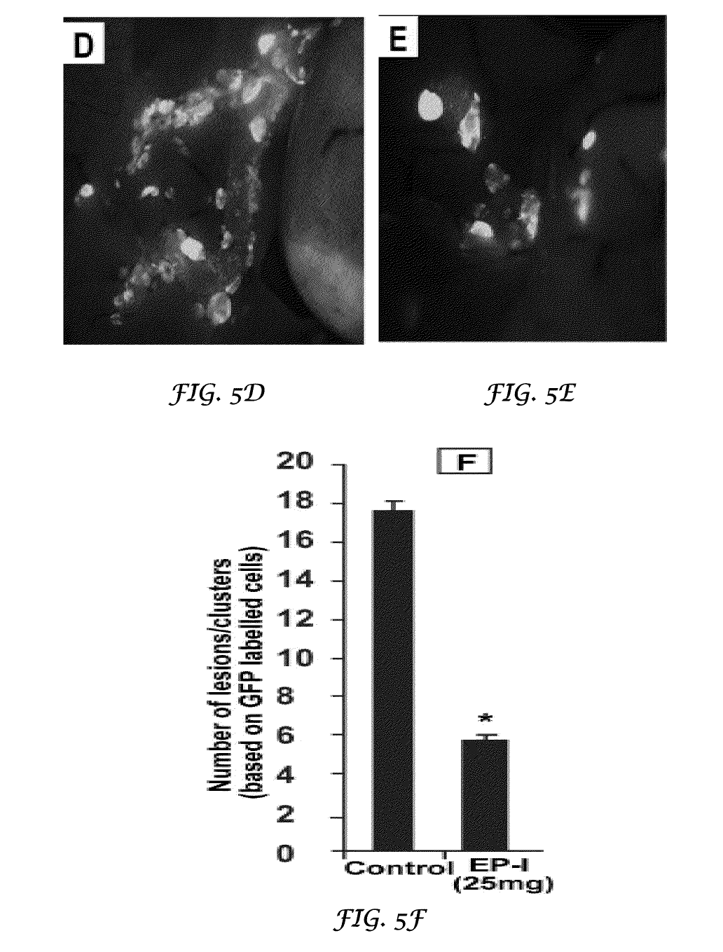

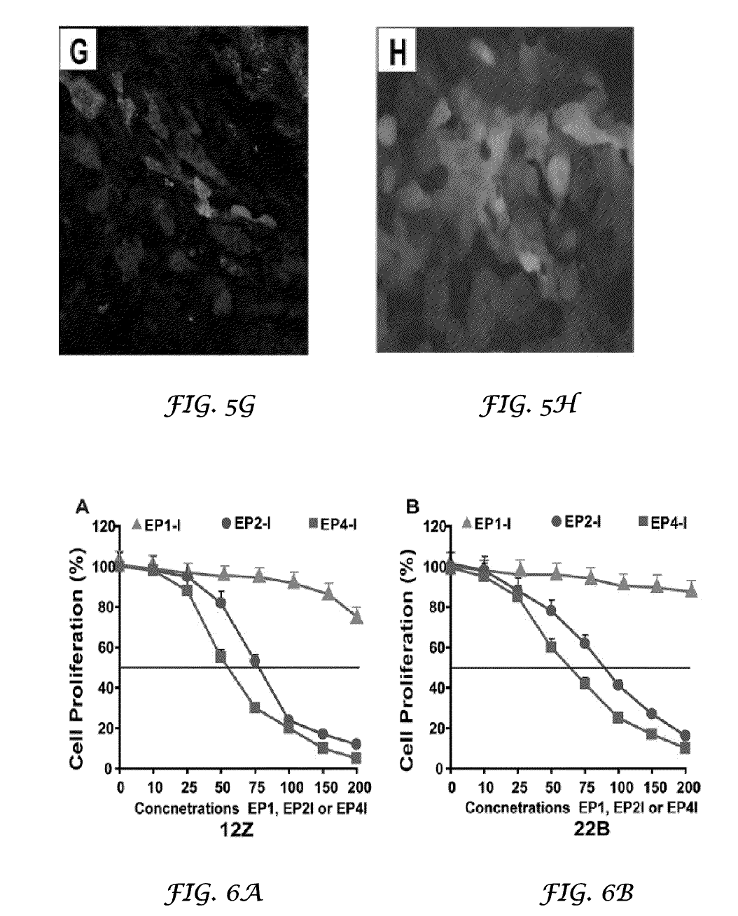

The inventors further determined whether xenograft of human endometriotic epithelial cells 12Z alone was able to survive and organize endometriosis glands. The inventors labeled 12Z cells with green fluorescent protein (GFP) and stable 12Z-GIN-GFP cells were established using lentiviral vector pCMV-GIN-ZEO (Open Biosystems). The pCMV-GIN-ZEO-GFP vector construction and transduction has been previously described (93). The day of xenograft is considered as day 0. In order to simulate clinical condition the 12Z-GIN-GFP cells were allowed to grow for minimum of 14 days to establish endometriosis. On day 15 post-xenograft the xenografted nude mice were divided into four groups based on 2×2 factorial design and received the same treatment regimen as described previously. All xenografted nude mice in each group were imaged on day 0 (before xenograft), 7, 21 and 28 post-xenograft. Survival and growth of epithelial cells 12Z-GIN-GFP was monitored using NightOWL II LB 983 Bioimager, and fluorescence dissection/confocal microscopy. Results (FIGS. 5A-5H) indicated that xenograft of endometriotic epithelial cells 12Z alone were not able to organize endometriosis glands suggest the requirement for stromal cell interactions in establishment of endometriosis. Interestingly, 12Z cells were able to proliferate, invade, and survive at the ectopic sites without stromal cell interaction for 28 days, suggest its solitary self-survival potential. Selective inhibition EP2/EP4 inhibitors at 10 mg and 25 mg inhibited (P<0.05) growth of 12Z cells ˜25% and ˜60%, respectively temporally within 14 days of treatment by contrast 5 mg did not have any effect.

The results of the animal model suggests that xenografts of human immortalized endometriosis epithelial and stromal cells into the peritoneal cavity of the recipient nude mice are able to proliferate, attach, invade, reorganize, and establish peritoneal endometriosis. Endometriosis glands at different stages of growth were present in induced endometriosis-like lesions. Proliferating cell nuclear antigen, metalloproteinase 2, estrogen receptor-a, cyclooxygenase-2, and prostaglandin E2 receptors EP2 and EP4 proteins were expressed in both endometriosis glandular epithelial and stromal cells of the induced endometriosis-like lesions.

Endometriosis is a common benign chronic gynecological disease of reproductive age women characterized by the presence of functional endometrial tissues outside the uterine cavity. More commonly, endometriosis lesions are found in the pelvic cavity/peritoneal organs where theses tissues respond to the menstrual hormonal changes and menses (1). The prevalence of this disease is ˜2-50% depending on the population of women studied and diagnostic methods used, and increases to 20-30% in women with subfertility and 40-60% in women with dysmenorrhoea or severe menstrual pain (2). Two major symptoms of endometriosis are intolerable pelvic pain and infertility, which profoundly affect the quality life in women of reproductive age (1, 2). Despite its high prevalence, pathogenesis of endometriosis is largely unknown. The most widely accepted theory is that the viable endometrial tissue fragments are refluxed through the oviducts into the pelvic cavity during retrograde menstruation (3). Endometriosis has been traditionally viewed as an estrogen-responsive disease (1, 4, 5); however, a recent report suggests that endometriosis is also a progesterone-unresponsive disease (6).

Prevalent treatment strategies include surgical interventions, medical therapy or a combination of both. After surgical removal of endometriosis lesions, the disease reestablishes within 3-5 years in ˜30-50% of women. Surprisingly, the disease reoccurs in ˜10% of women who had uterus and both ovaries removed (7). Hormonal therapy to induce a hypoestrogenic state through the use of oral contraceptives, progestagens, and gonadotropin releasing hormone analogs and androgenic agents can be prescribed only for a short time due to unacceptable side-effects, pseudomenopause and bone density loss in reproductive age women (1, 2, 7). Nevertheless, the recurrence rate is ˜50-60% after cessation of therapy within a year (7). Further, two apparently expensive unsuccessful clinical trials on the use fulvestrant—an estrogen receptor antagonist and raloxifene—a selective estrogen receptor modulator to inhibit estrogen actions for the treatment of endometriosis in women were discontinued due to unfavorable outcomes (7). Together, existing treatment modalities fail to prevent reoccurrence of disease, and affect pregnancy and reproductive health of women. This suggests a crucial need to identify potential cell signaling pathways for targeted therapies, including non-estrogen targets, for endometriosis.

Lack of information on molecular endocrinology of human endometriotic cells remains one of the major limitations to identify potential targeted therapies for this disease (7, 8). A growing body of evidence indicates that prostaglandins (PGs) contribute to the pathophysiology/pathogenesis of endometriosis (9-14). Concentrations of PGE₂ in peritoneal fluid are higher in women suffering from endometriosis compared to disease-free women (15), and this increased PGE₂ is considered to be involved in endometriosis-associated pain (9). The inventors and other researchers have previously shown that COX-2 is more abundantly expressed in ectopic endometriotic tissues compared to eutopic endometrial tissues during the menstrual cycle in women (11, 13, 14). A placebo-controlled double-blinded study reported that selective COX-2 inhibitor rofecoxib at 25 mg/day for six months effectively suppressed the pelvic pain symptoms in endometriosis patients in Europe (16). However, no clinical trial has been approved to test the use of COX-2 inhibitors for the treatment of endometriosis in women in the United States. In animal model for endometriosis, selective COX-2 inhibitor celecoxib decreased establishment of endometriosis and number and size of endometriotic implants in rat model (17), and selective COX-2 inhibitor NS-398 induced regression of endometriotic implant through caspase-3 dependent apoptosis in hamster model (10). However, nonselective or partially selective COX-2 inhibitor nimesulide failed to decrease number and size of endometriotic lesions in nude mice model (18), and the lack of effect could be due to low dose and duration of treatment and ability of nimesulide to inhibit COX-2 activity in endometriotic implants compared to celecoxib or NS-398 (19). In vitro studies indicated that PGE₂ regulates genes involved in steroid biosynthesis and estradiol production and in turn estradiol increases COX-2 derived PGE₂ production by establishing positive loop between PGE₂ and estradiol in primary cultured endometriotic stromal cells and endometriosis lesions per se in human (20-22). Celecoxib prevents growth and survival of primary cultured eutopic endometrial epithelial cells from endometriosis patients (23). The inventors recently discovered that selective inhibition of COX-2 using NS-398 prevents survival, migration and invasion of human immortalized endometriotic epithelial cells 12Z and stromal cells 22B which is associated with decreased PGE₂ production (11). Collectively, these studies strongly indicate a prerequisite for COX-2/PGE₂ in pathogenesis of endometriosis and inhibition of COX-2/PGE₂ could emerge as a potential therapy for this disease in human.

Ablation of the COX-2 gene resulted in multiple reproductive failures in mice (24) and therapeutic use of COX-2 inhibitors resulted in undesirable cardiovascular side effects (25). There is a need to discover alternative targets down-stream of COX-2 to inhibit actions of PGE₂ selectively at its receptors level. Multifaceted actions of PGE₂ are primarily mediated through G-protein coupled membrane receptors designated EP that includes EP1, EP2, EP3 and EP4 (26). EP2 and EP4 receptors are coupled to Gs proteins that activate adenylate cyclase and generates cAMP which in turn activates the protein kinase A pathway (26). One of the fascinating aspects of EP2 and EP4 signaling is its prone cross-talk with the EGFR and β-catenin pathways through PKA-dependent or -independent intracellular pathways (25, 27-31). The EP1 receptor is coupled to Gq protein and activates phospholipase C that results in generation of two second messengers inositol triphosphate (IP₃) which liberates intracellular calcium (Ca²⁺) and diacylglycerol that activates protein kinase C (26). There are four EP3 isoforms EP3A, EP3B, EP3C and EP3D coupled to Gq, Gs, and G1 proteins. Activation of EP3 receptors produces a wide range of complex and even opposite actions from inhibition or induction of cAMP production to increases in Ca²⁺ and IP₃ (26). The EP2 and EP4 receptors are coupled to Gs proteins and activate three major signaling pathways: [1] interact with adenylate cyclase and generate cAMP, which in turn activates the protein Q1 kinase A signaling pathway (26), [2] interact with SRC kinase intracellularly through protein kinase A-dependent or -independent pathways (24, 25, 27-28, 31) and constitutively transactivate epidermal growth factor (EGF) receptor without the involvement of EGF receptor ligand, which in turn activates mitogen-activated protein kinase Q2 and phosphoinositide 3-kinase/AKT pathways, and [3] transactivate β-catenin signaling pathways intracellularly through axin and AKT pathways without contribution of the Wnt ligand in many invasive cell types (24, 25, 27-28, 31).

PGE₂ is an important anti-apoptotic mediator and prevents cells from undergoing programmed cell death or apoptosis by activating several cell survival and anti-apoptotic pathways (25, 32-34). Human endometriotic cells are resistant to apoptosis and this plays an intrinsic role in establishment and growth of endometriosis lesions in women (35-37). It has been proposed that medical strategies to intervene antiapoptotic or proapoptotic pathways may lead to identify effective treatment modalities for the treatment of endometriosis in women (35-37). Hence, understanding of functional association between PGE₂ signaling and endometriotic cell apoptosis and survival will not only advance the current understanding of pathophysiology of endometriosis but also lead to identify new specific signaling modules to inhibit growth and survival of endometriosis lesions in women.

In addition to providing an understanding of the molecular and cellular aspects of the pathogenesis of endometriosis in humans, the present invention addresses the following issues: (i) determining the PGE₂ signaling network supporting the survival of human endometriotic cells; (ii) inhibiting the PGE₂ receptors to be used as potential molecular targets to induce apoptosis of human endometriotic cells; and (iii) determining the spatial expression of PGE₂ signaling components in ectopic endometriotic and eutopic endometrial tissues from women during the menstrual cycle. The results obtained from the various studies conducted in the present invention indicate that PGE₂ promotes survival of human endometriotic cells through EP2 and EP4 receptors by transactivating EGFR, AKT, ERK1/2, NFκB, and β-catenin pathways, and selective combined inhibition of EP2 and EP4 induces apoptosis of human endometriotic cells by compromising these survival pathways and activating intrinsic apoptosis pathways.

Materials: The reagents used in studies conducted as a part of the present invention were purchased from the following suppliers: Prestained protein markers and Bio-Rad assay reagents and standards (Bio-Rad Laboratories, Hercules, Calif.); Protran BA83 Nitrocellulose membrane (Whatman Inc, Sanford, Me.); Pierce ECL and mitochondria isolation kit (Pierce Biotechnology, Rockford, Ill.); protease inhibitor cocktail tablets complete EDTA-free and PhosStop (Roche Applied Biosciences, Indianapolis, Ind.); antibiotic-antimycotic, and trypsin-EDTA, Alexa Fluor 488, APO-BrdU TUNEL assay kit, and ProLong Gold antifade reagent (Invitrogen Life Technologies Inc, Carlsbad, Calif.); Vectastain Elite ABC kit (Vector Laboratories Inc, Burlingame, Calif.); Blue X-Ray film (Phenix Research Products, Hayward, Calif.); fetal bovine serum (HyClone, Logan, Utah); Lab-Tek II chamber slides (Nunc, Rochester, N.Y.), and tissue culture dishes and plates (Corning Inc, Corning, N.Y.). EP2 and EP4 siRNA, siGLORISC-free siRNA and DharmaFect-1 were obtained from Dharmacon Inc, Lafayette, Colo. Antagonists/inhibitors for EP1 (SC19220), EP2 (AH6809), EP4 (AH23848) and EP1, EP2, EP3 and EP4 antibodies were purchased from Cayman Chemicals (Ann Arbor, Mich.). All other antibodies used in this study were purchased from Cell Signaling Technology (Danvers, Mass.) or Santa Cruz Biotechnology (Santa Cruz, Calif.) except β-actin monoclonal antibody (Sigma-Aldrich, St-Louis, Mo.), goat anti-rabbit or anti-mouse IgG conjugated with horseradish peroxidase (Kirkegaard & Perry Laboratories, Gaithersburg, Mass.). The chemicals used were molecular biological grade from Fisher Scientific (Pittsburgh, Pa.) or Sigma-Aldrich (St. Louis, Mo.).

Endometriotic and Endometrial Cell Lines and Culture: Clinically, endometriosis lesions are classified into red, white, and bluish-black lesions. The red lesions are highly vascularized and extremely proliferative, adhesive, invasive, and represent the active/progressive phase of the disease (52, 56, 57). Immortalized human endometriotic epithelial cells 12Z and stromal cells 22B used in this study were derived from these active red lesions of peritoneal endometriosis from women, and these share several phenotypic and molecular characteristics of primary cultured endometriotic cells (58). Information accumulated from previous studies conducted by the inventors indicates that these endometriotic cells mimic the active/progressive phase of the diseases and a potential tool to develop targeted therapy (7, 8, 11, 39, 58-61). The inventors have previously reported that molecular and cellular behavior of these 12Z and 22B cells differ from those of human eutopic endometrial epithelial cells (HES) and stromal cells (HESC) from endometriosis-free woman in various aspects. Endometriotic cells 12Z and 22B produced large amounts of PGE₂, genes associated with cytokine and growth factor were more abundantly expressed, and 12Z and 22B cells were highly migratory and invasive compared to HES and HESC (11, 39). The inventors further compared molecular and cellular behavior of 12Z and 22B cells with available information from primary cultured endometriotic cells and eutopic endometrial cells from endometriosis women, and endometriosis lesions in women and animal models for endometriosis, indicating that 12Z and 22B cell possess several similarities with their counterparts (39, 58, and the references cited in). Interestingly, xenograft of a mixed population of these 12Z and 22B cells into the peritoneal cavity of nude mice is able to proliferate, attach, invade, reorganize and establish peritoneal endometriosis-like lesions, and histomorphology of these lesions are similar to that of spontaneous peritoneal endometriosis lesions in women (8). These 12Z and 22B cells expressed COX-2, EP2 and EP4 proteins abundantly in vitro (11, 39) and in vivo in induced endometriosis-like lesions in nude mice (8). These results together suggest that human endometriotic cells 12Z and 22B is an ideal model system to study PGE₂ signaling in pathogenesis of endometriosis in women.

These well-characterized 12Z and 22B cells were cultured in DMEM/F12 without special steroid treatment containing 10% fetal bovine serum (FBS) and penicillin (100 U/ml), streptomycin (100 μg/ml) and amphotericin-B 2.5 μg/ml in a humidified 5% CO₂ and 95% air at 37° C. as we described previously (11, 39). At 70-80% confluency the cells were cultured in DMEM/F12 with 2% dextran-charcoal-treated fetal bovine serum (DC-FBS) and treated with EP inhibitors (EP-I) for EP1 (SC19220-100 100 μM), EP2 (AH6809-75 μM) and/or EP4 (AH23848-50 μM) for 24 h.

EP2/EP4 small interfering RNA (siRNA): 12Z and 22B cells (3.0×10⁵/well) were cultured in antibiotic-free DMEM/F12 with 10% FBS in six-well tissue culture plates. At 70-80% confluency, cells were used for EP2, EP4 or EP2/EP4 knock-down experiments using SMART-pool siRNA duplex delivered by DharmaFect-1 as described previously (11) and per manufacturer's instructions (Dharmacon Inc, Lafayette, Colo.). As an internal control, MOCK siRNA was used. According to the manufacturer's instructions, SMARTpool siRNA were consisted of at least four individual siRNA duplexes targeted against a specific gene and designed using a bioinformatics technology known as SMARTselection. This resulted in the generation of siRNAs more than 97% of the time, and the targeted message level was reduced by more than 70% with in 24 hours after transfection. The inventors used SMARTpool siRNA in order to use more than one siRNA duplex from different regions of the gene of interest to avoid non-specific indirect effects of single siRNA duplex to a single region of the target gene. Briefly, siRNA duplexes (100 nM/well) and DharmaFect-1 (3 μl/well) were diluted in 50 μl antibiotic and serum-free DMEM/F12 medium separately and mixed gently and incubated for 5 min at room temperature. Afterwards, EP2, EP4 or EP2/EP4 siRNA and DharmaFect-1 were mixed (total volume 100 μl) and incubated at room temperature for 20 min. Then, 100 μl siRNA: DharmaFect-1 complex was diluted with 2 ml antibiotic-free media with 10% FBS and added to the well. After 24 h, the medium was replaced with fresh DMEM/F12 with 10% FBS and incubated for 24 h. Fluorescence labeled siGLO RISC-free siRNA was transfected separately and transfection efficiency was estimated using a fluorescence microscope. Transfection efficiency more than 80% was considered as optimal conditions for further experiments. Efficiency of siRNA on silencing of EP2 and EP4 genes and proteins was assessed by RT-PCR and western blot, respectively 72 h post-transfection knock-down efficiency was 70-80% in both 12Z and 22B cells.

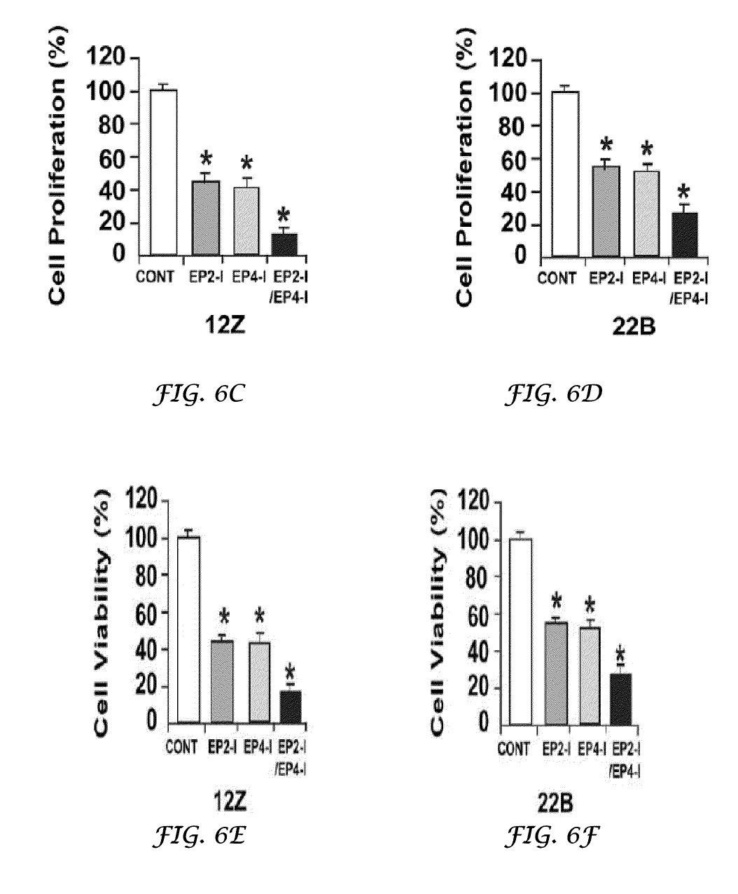

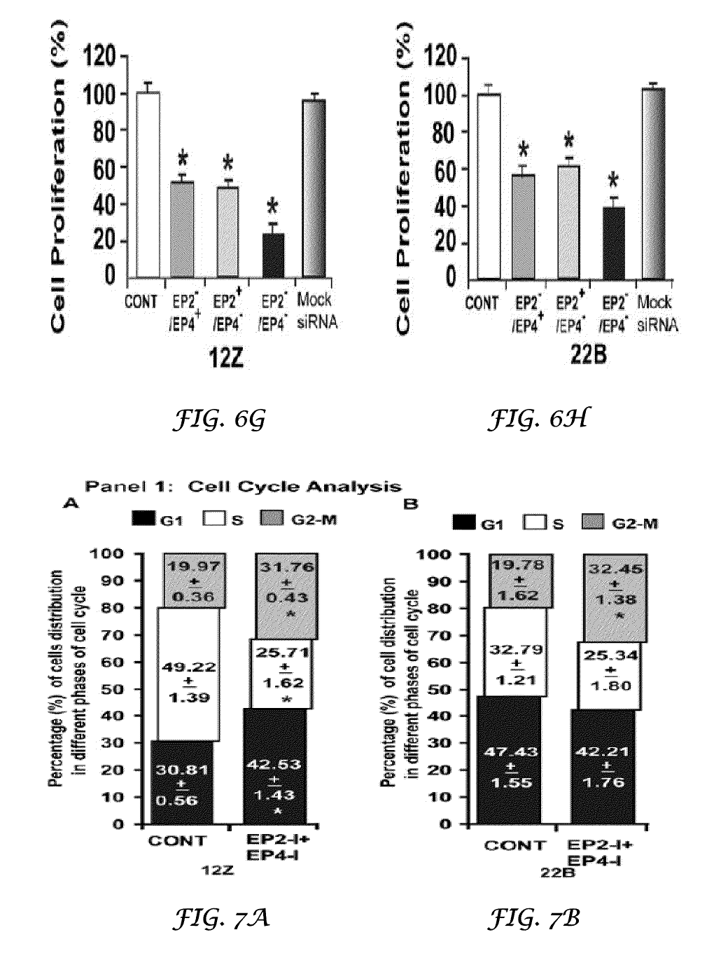

Cell Proliferation Assay: The 12Z and 22B cells (1×10⁵/well) were cultured in DMEM/F12 with 10% FBS in six-well plates. At 70%-80% confluency the cells were cultured in DMEM/F12 with 2% dextran charcoal-treated FBS and treated with inhibitors for EP1 (SC19220; 100 mM), EP2 (AH6809; 75 mM), EP4 (AH23848; 0 mM), or a combination of EP2 (AH6809; 75 mM) and EP4 (AH23848; 0 mM) for 36 hours. The concentrations for each inhibitor was determined by dose-response studies using 0, 10, 25, 50, 75, 100, 150, and 200 mM and an optimal concentration was selected based on its effects on proliferation of human endometriotic cells (FIGS. 6A-6B). These inhibitors competitively bind with respective receptors and inhibit their functions (40-42). For the siRNA study, 24 hours after transfection of siRNA the medium was replaced and the cells were cultured in 2% dextran charcoal-treated FBS, which was considered as time 0 hour, and cell proliferation was estimated at 36 hours. The number of cells was counted using a Coulter counter (11) and considered as 100% present in control. Data were expressed as mean±SEM of three independent studies conducted in duplicate.

Cell Viability Assay: The 12Z and 22B cells (1.25×10⁴ cells/well) were cultured in DMEM/F12 with 10% FBS in 96-well plates and treated as described previously. At the end of the experiments the cell viability (metabolic activity) was determined using a cell proliferation assay kit WST-1 according to the manufacturer's (Roche Applied Sciences) instructions. Metabolic activity recorded in control cells was considered as 100%. Data were expressed as mean±SEM of three independent experiments conducted in triplicate.

Cell Cycle Analysis: The 12Z and 22B cells were cultured in T-75 flasks and treated as described previously. The cells were first fixed in 1% buffered paraformaldehyde saline for 15 minutes on ice, and then fixed in ice cold 70% ethanol and kept at −20° C. for 30 minutes. The cells were rehydrated in phosphate-buffered saline (PBS) for 15 minutes, treated with DNase-free RNase (100 mg/mL), and stained with propidium iodide (25 mg/mL) in staining buffer (100 mM Tris, pH 7.4, 150 mM NaCl, 1 mM CaCl₂, 0.5 mM MgCl₂, 0.1% Nonidet P-40) for 30 minutes at room temperature. The number of cells distributed in G1, S, and G2-M phases of the cell cycle was determined by fluorescence-activated cell sorter (FACS) analysis of propidium-stained cells distribution using a flow cytometer (FACSCaliber; Becton Dickinson, San Jose, Calif.) and ModFit LT program (Verity Software House) at TAMU Health Science Center. Data were expressed as mean±SEM of three independent experiments conducted in triplicate.

Protein extraction and western blot: Total protein was isolated from cells and immunoblotting cyclins A, B1, D1, D2, D3, E2, and cyclin-dependent kinases (CDKs) CDK1, CDK2, CDK4, CDK6, and CDK inhibitors p15, p16, p21, p27 was performed as described by the present inventors previously (11, 81). Briefly, the cells were harvested using 1% Trypsin-EDTA and pelleted. The cell lysates were sonicated in sonication buffer which consisted of 20 mM Tris-Hcl, 0.5 mM EDTA, 100 μM DEDTC, 1% Tween, 1 mM phenylmethylsulfonyl fluoride, and protease inhibitor cocktail tablets: complete EDTA-free (1 tablet/50 ml) and PhosStop (1 tablet/10 ml). Sonication was performed using a Microson ultrasonic cell disruptor (Microsonix Incorporated, Farmingdale, N.Y.). Protein concentration was determined using the Bradford method (63) and a Bio-Rad Protein Assay kit. Protein samples (75 μg) were resolved using 7.5%, 10% or 12.5% SDS-PAGE. Chemiluminescent substrate was applied according to the manufacturer's instructions (Pierce Biotechnology). The blots were exposed to Blue X-Ray film and densitometry of autoradiograms was performed using an Alpha Imager (Alpha Innotech Corporation, San Leandro, Calif.).

Mitochondria/cytosol isolation: 12Z and 22B cells were cultured, treated and harvested as described above. The mitochondrial and cytosolic fractions were isolated using mitochondria isolation kit for cultured cells according to the manufacturer's instructions (Pierce Biotechnology). Cytosolic fractions were concentrated by Amicon Ultra 3K column (Millipore). Both protein fractions were briefly sonicated. Protein concentration was determined using the Bradford method (63) and a Bio-Rad Protein Assay kit.

Immunoprecipitation: 12Z and 22B cells were cultured, treated, harvested, and then total cell lysates were prepared as described above. Total cell lysate (1 mg) was precleared by incubating with appropriate preclearing matrix (Santa Cruz Biotechnology) for 30 min at 4° C. The precleared cell lysate was incubated with primary antibody overnight at 4° C. at the recommended concentrations given by manufacturers (Cell Signaling Technology and/or Santa Cruz Biotechnology), and then further incubated with immunoprecipitation matrix-ExactraCruz (Santa Cruz Biotechnology) overnight at 4° C. Protein-antibody complexes were precipitated using protocols provided by Santa Cruz Biotechnology and/or Cell Signaling Technology.

Immunofluorescence: 12Z and 22B cells were seeded at 50,000 cells per well on Lab-Tek II chambered slides and cultured as described above. At 70-80% confluency the cells were treated with EP2 and EP4 inhibitors as described above. The procedure given by Cell Signaling Technology was followed with minor modifications. Cells were rinsed in PBS, fixed in 1% paraformaldehyde for 15 min at room temperature, and permeabilized for 10 min in 100% methanol at −20° C. Cells were blocked for 1 h in 10% normal serum from same species in which secondary antibody was developed and then incubated overnight with primary antibodies at the concentrations recommended by manufacturer. For the negative control, serum or IgG from respective species with reference to the primary antibody at the respective dilution was used. After washing in 0.2M PBS/0.3% Tween, cells were incubated with Alexa Fluor 488 conjugated secondary antibodies for 1 h. Cells were washed and mounted with ProLong Gold antifade reagent. Images were visualized by using digital imaging and an image analysis workstation consisting of a Zeiss Axioplan 2 Research Microscope interfaced with a Zeiss Axiocam HR high resolution color CCD camera with Zeiss Axiovision (Carl Zeiss, Thornwood, N.Y.).

Terminal deoxynucleotide transferase dUTP nick end labeling (TUNEL) assay: Non-adherent and adherent cells were harvested, mixed together, and resuspended at the concentrations of 1×10⁶ cells/ml. Nicks in the DNA were determined by terminal deoxynucleotidyl transferase (TdT) and 5-bromo-2′-deoxyuridine 5′-triphosphate (BrdUTP) labeling using APO-BrdU TUNEL Assay Kit. Detection of BrdU incorporation at DNA break sites was achieved through Alexa Fluor 488 dye-labeled anti-BrdU antibody. The staining procedures were performed as recommended by manufacturers. Numbers of apoptotic cells were analyzed by a flowcytometer (FACSCaliber, Becton Dickinson, San Jose, Calif.) using Cell Quest software. In addition, the presence of TUNEL labeled DNA fragments were determined by fluorescence microscopy. Digital images were captured using a Zeiss Axioplan 2 Research Microscope with an Axiocam HR digital camera.

Endometriosis and endometrial tissues: The following tissues were collected from women presented at Obstetrics and Gynecology Unit, and processed at Anatomic Pathology Laboratory for diagnostic purposes, Scott & White Memorial Hospital, Texas A&M University System Health Science Center. Ectopic endometria from peritoneal lesions (endometriotic tissue, n=12) were collected from women with endometriosis. Eutopic endometria (n=12) were collected from women with endometriosis. Normal eutopic endometria (n=12) were collected from endometriosis-free women undergoing hysterectomy for benign gynecological indications. Each of the women reported regular menstrual cycles (25-40 days cycle length), and no hormonal medication in the last 3 months. All the endometriotic and endometrial tissues were collected from proliferative phase of the menstrual cycle that was confirmed by patient's last menstrual period, progesterone profile, and histology of endometriotic and endometrial tissues. Ectopic endometria were classified I-IV stages based on criteria established by the American Society for Reproductive Medicine (57). Stages I and II were included in this investigation. For the present study, additional sections were cut from these archived paraffin-embedded tissues, which were not needed for patient care.

Immunohistochemistry: Ectopic and eutopic endometrial tissue sections were fixed in 4% buffered paraformaldehyde saline for 4 h at 4° C., and processed using standard procedures (11, 62). Paraffin sections (5 μm) were used for immunohistochemical localization of proteins involved in PGE₂ signaling using a Vectastain Elite ABC kit as previously we described (11, 62) according to the manufacturer's protocols. The tissue sections were incubated with specific antibodies at the concentrations recommended by manufacturers overnight at 4° C. Then, tissue sections were further incubated with the secondary antibody (biotinylated IgG) for 45 min at room temperature. For the negative control, serum or IgG from respective species with reference to the primary antibody at the respective dilution was used. Digital images were captured using a Zeiss Axioplan 2 Research Microscope (Carl Zeiss, Thornwood, N.Y.) with an Axiocam HR digital camera. The intensity of staining for each protein was quantified using Image-pro Plus as described previously by the inventors (62) according to the manufacturer's (Media Cybernetics, Inc; Bethesda, Md.) instructions. The inventors preferred immunohistochemistry followed by densitometry compared to western blot for the following reasons: (i) quantity of endometrial and endometriotic tissues obtained from each patient is not to enough to extract adequate proteins and to analyze various proteins using western blot; and (ii) immunohistochemistry is being used as a primary technique to confirm endometriosis (presence of endometriosis glands lined by epithelial cells and surrounded by stromal cells) in women clinically; therefore, immunohistochemistry will provide details on spatial expression of a specific protein in glandular epithelial and stromal cells of endometriosis while western blot only provide information on total steady state expression levels of a particular protein in the given tissue.

Statistical analyses: Statistical analyses were performed using general linear models of Statistical Analysis System (SAS, Cary, N.C.). The effects of inhibition of EP receptors on cell proliferation and expression levels of different proteins in 12Z and 22B cells in vitro, relative spatial expression of different proteins in glandular epithelium and stroma in ectopic and eutopic endometria in vivo, and the effects of inhibition of EP receptors on cell apoptosis and expression levels of different proteins in 12Z and 22B cells in vitro were analyzed by one-way analysis of variance (ANOVA) followed by Tukey-Kramer HSD test. The relationship between number of endometriotic cells present and their viability were determined by simple linear correlation. The numerical data are expressed as mean±SEM. Statistical significance was considered at P<0.05.

Inhibition of EP2/EP4 signaling decreased endometriotic cell proliferation and viability: The 12Z and 22B cells produce large amounts of PGE₂ and express EP receptors at basal condition (11, 70). Therefore, the inventors pharmacologically inhibited EP1, EP2, and EP4 receptor signaling. Results (FIGS. 6A-6B) indicated that pharmacologic inhibition of EP2 or EP4 decreased (P<0.05) proliferation of 12Z and 22B cells in a dose dependent manner. In contrast, inhibition of EP1 did not decrease proliferation of 12Z and 22B cells, and therefore, its role was not further examined. Based on these results the present inventors selected doses of 75 μM and 50 μM, respectively, for EP2 and EP4 inhibitors. These results suggest that selective blockade of EP2 and EP4 inhibits proliferation of 12Z and 22B cells through suppression of specific EP2- and EP4-mediated signaling pathways but not due to nonspecific or toxic effects. The role of EP3 was not examined considering the low expression levels in eutopic and ectopic endometria (8, 71). Furthermore, the results (FIGS. 6C-6D) indicated that inhibition of either EP2 or EP4 signaling decreased (P<0.05) cell proliferation ˜50%-55% in 12Z cells and 40%-45% in 22B cells. Interestingly, combined inhibition of EP2 and EP4 produced additive effects and decreased cell proliferation ˜85%-88% in 12Z cells and ˜70%-75% in 22B cells. In addition, results (FIGS. 6E-6F) of cell viability demonstrated that inhibition of either EP2 or EP4 decreased (P<0.05) cell viability ˜55% in 12Z cells and ˜45% in 22B cells Inhibition of both EP2 and EP4 decreased (P<0.05) cell viability ˜80% in 12Z cells and 68% in 22B cells. The percentage of cell viability was positively correlated (r=0.98) with number of cells present in control and treatment groups. Furthermore, results (FIGS. 6G-6H) of siRNA indicated that silencing of either EP2 or EP4 genes decreased (P<0.05) cell proliferation ˜45%-50% in 12Z cells and 40%-42% in 22B cells. Interestingly, double knocked-down of EP2 and EP4 genes resulted in additive effects and decreased (P<0.05) cell proliferation ˜75% in 12Z cells and ˜65% in 22B cells. Mock siRNA was used as an internal control, which did not affect proliferation of 12Z and 22B cells. The inventors have already shown that EP2 and EP4 genes were knocked-down up to ˜80% in 12Z and 22B cells (70).

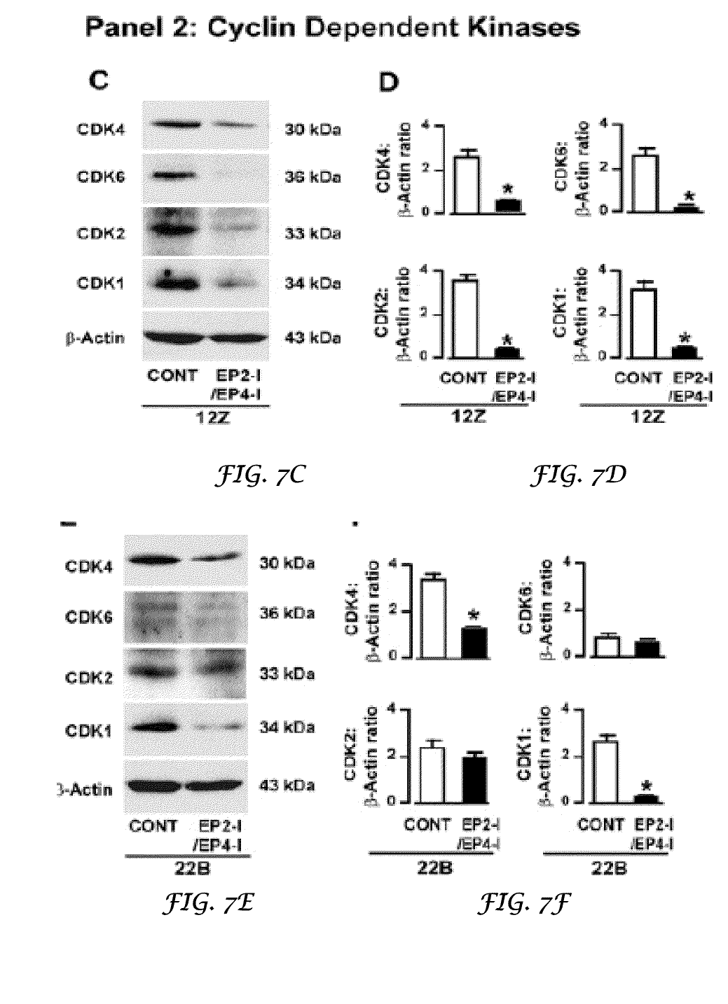

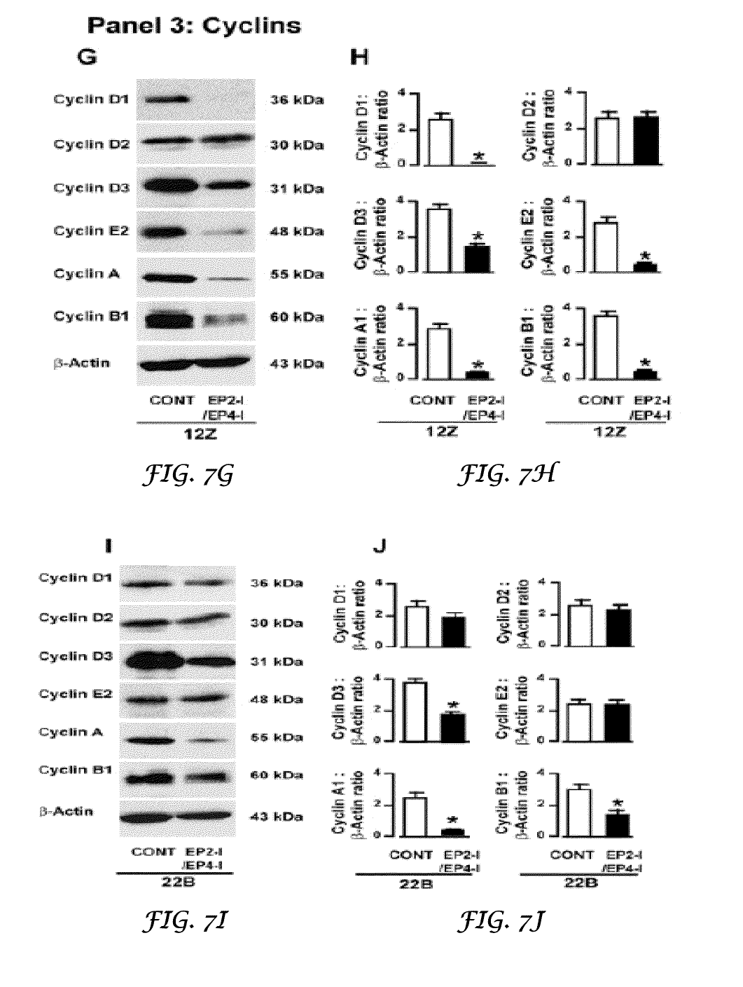

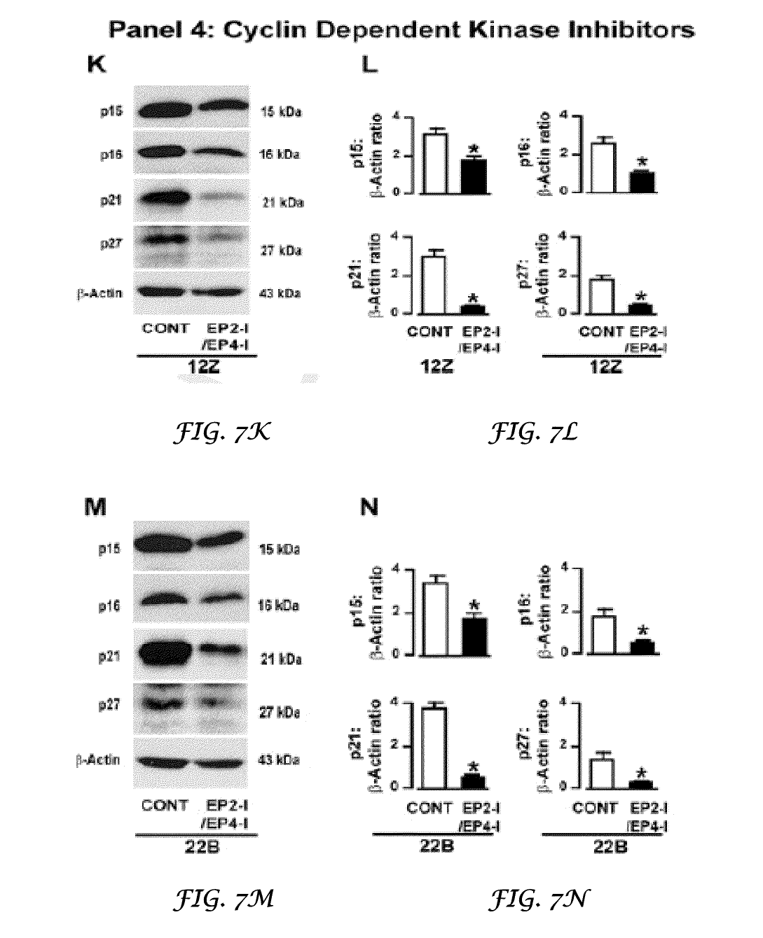

Interactions between EP2/EP4 signaling and cell cycle regulation in Endometriotic Cells: Results indicated that inhibition of EP2/EP4 signaling arrested (P<0.05) progression of cells through G1-S and G2-M phases of the cell cycle in 12Z cells, but it arrested (P<0.05) only G2-M progression in 22B cells (FIGS. 7A and 7B). Next, to get additional insight into growth inhibitory mechanisms in endometriotic cells, the inventors elucidated the association between EP2/EP4 signaling and cell cycle regulatory proteins. Results indicated that: [1] inhibition of EP2 and EP4 signaling down-regulated (P<0.05) CDK4, CDK6, CDK2, and CDK1 proteins in 12Z cells and down-regulated (P<0.05) only CDK4 and CDK1, but not CDK2 and CDK6, proteins in 22B cells (FIG. 7C-7F); [2] inhibition of EP2 and EP4 signaling down-regulated (P<0.05) cyclin D1, D3, E2, A, and B1, but not cyclin D2, proteins in 12Z cells and down-regulated (P<0.05) only cyclin D3, A, and B1, but not D1, D2, and E2, proteins in 22B cells (FIG. 7G-7J); and [3] inhibition of EP2 and EP4 signaling did not up-regulate CDK inhibitors P15, P16, P21, and P27 proteins but their expression levels were decreased (P<0.05) in both 12Z and 22B cells (FIGS. 7K-7N).

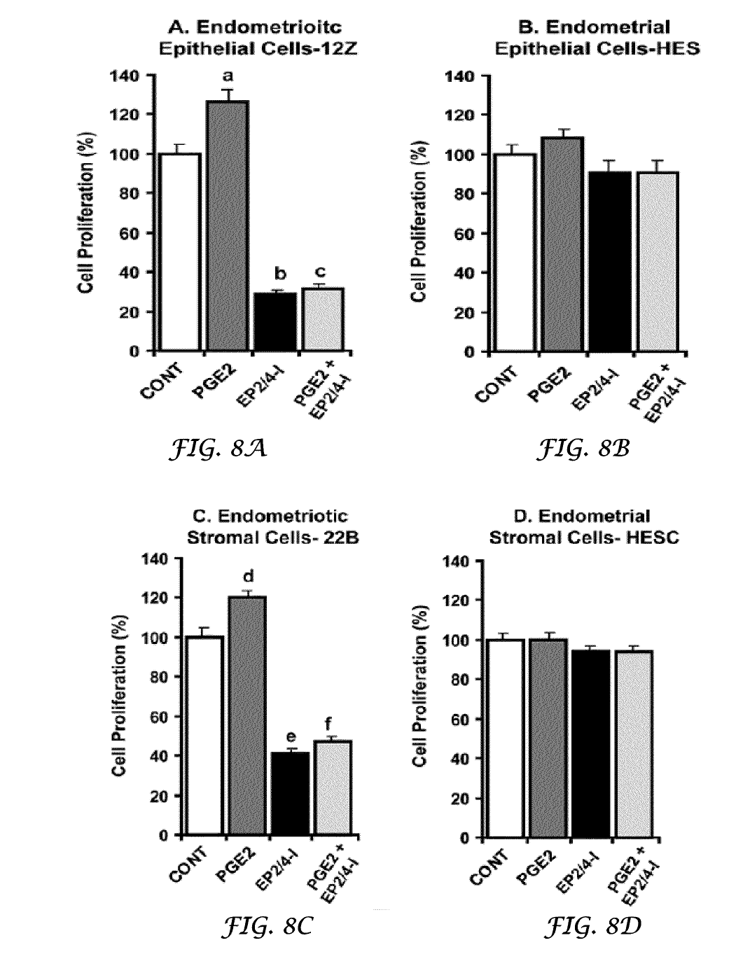

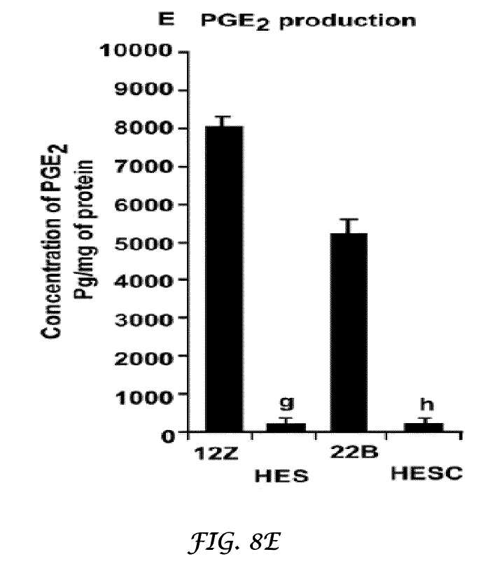

Inhibition of EP2/EP4 signaling did not decrease eutopic endometrial cell proliferation: The present inventors evaluated the effects of selective inhibition of EP2 and EP4 on eutopic HES and HSTR cells from endometriosis/disease-free women, and compared with endometriotic cells 12Z and 22B. Results (FIGS. 8A-8E) indicated that selective inhibition of EP2 and EP4 did not decrease proliferation of HES and HESC cells compared with 12Z and 22B cells. Furthermore, HES and HESC cells did not produce PGE₂, in contrast 12Z produced 7,500-8,000 pg/mg protein and 22B cells produced 5,000-6,000 pg/mg protein at basal condition (in the absence of any treatment). These results suggest that endometriotic epithelial and stromal cells, but not eutopic endometrial epithelial or stromal cells, highly depend on EP2- and EP4-mediated PGE₂ signaling for proliferation/growth.

These results of these studies together indicated that PGE₂ promotes survival of human endometriotic cells 12Z and 22B primarily through EP2 and EP4 receptors, and selective inhibition of EP2 and EP4 receptors induced apoptosis of 12Z and 22B cells. Further, these results suggest the existence of a compensatory mechanism between EP2 and EP4 receptors in mediating PGE₂ signaling in human endometriotic cells, and combined inhibition of both EP2 and EP4 is obligatory to understand PGE₂ signaling in the pathogenesis of endometriosis in human. Based on these data, pharmacological inhibition of both EP2 and EP4 signaling was further evaluated considering therapeutic use of EP2 and EP4 inhibitors for the treatment of endometriosis.

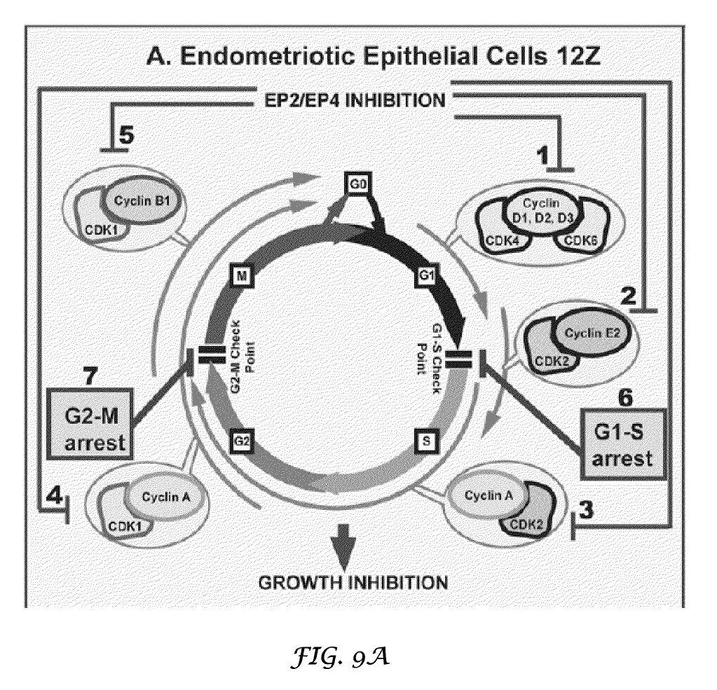

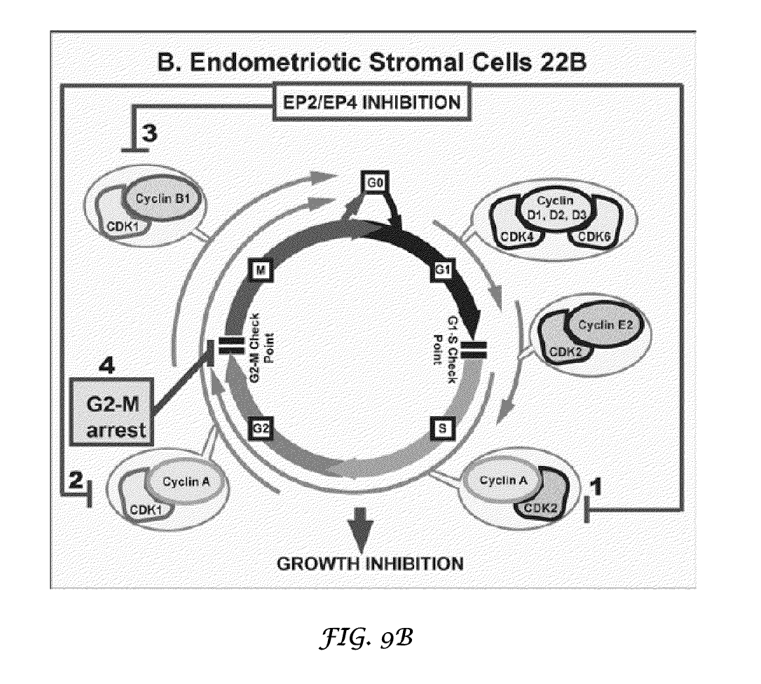

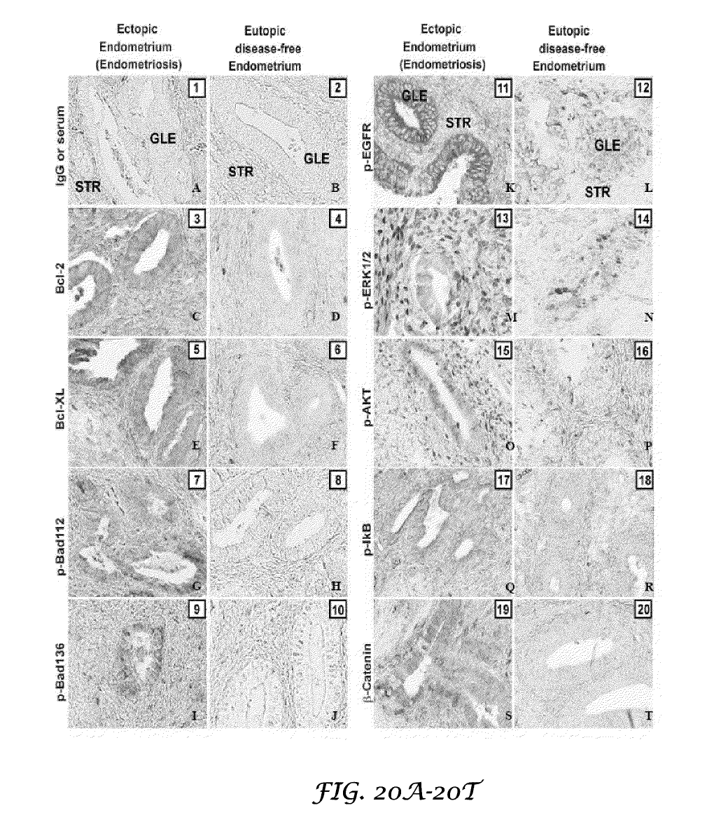

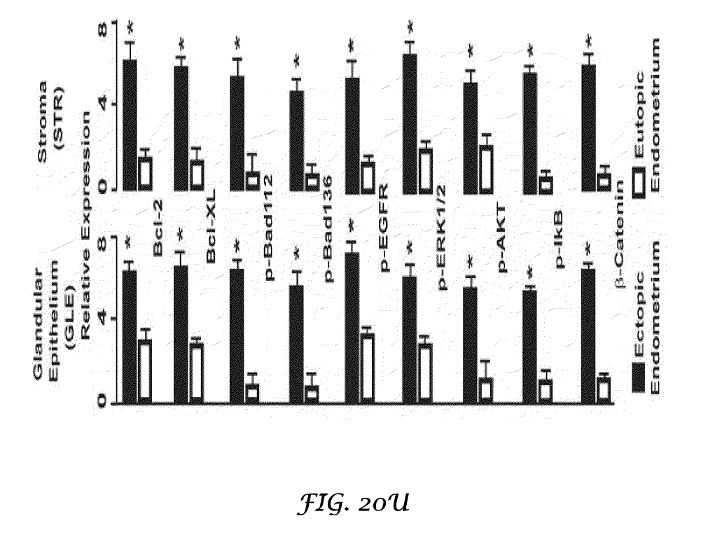

Selective inhibition of EP2- and EP4-mediated signaling regulates proliferation of human endometriotic cells. Data from studies conducted by the present inventors indicate that selective inhibition of EP2 and EP4 signaling decreases proliferation of human endometriotic epithelial cells 12Z through G1-S and G2-M checkpoint arrest and stromal cells 22B through G2-M checkpoint arrest. The epithelial-stromal-specific cell cycle arrest at G1-S and G2-M is associated with regulation of respective CDKs and cyclins. Selective CDK/cyclin complexes are activated at different phases/checkpoints of the cell cycle (FIGS. 9A-9B). Cyclin D1/D2/D3 and CDK4/6 complexes are activated in early to mid-G1 phase; cyclin E/CDK2 complexes are required for the G1/S transition; cyclin A/CDK2 complex is essential for the progression of S-phase/DNA synthesis; and cyclin A-B/CDK1 is necessary for G2-M transition (72-75). As proposed in FIGS. 20A and 20B, in response to selective inhibition of EP2 and EP4, down-regulation of cyclin D1-D3/CDK4, cyclin E2/CDK2, cyclin A/CDK2, and A-B/CDK1 complexes might be responsible for deregulated progression of cells through G1-S and G2-M checkpoints in endometriotic epithelial cells 12Z. In contrast, down-regulation of cyclin A-B/CDK1 complexes could explain G2-M checkpoint arrest in endometriotic stromal cells 22B in response to selective inhibition of EP2 and EP4. Furthermore, G1-specific cyclin D3 and CDK4 are decreased in 22B cells; however, the other G1-specific D1-D2/CDK6 complexes are not down-regulated. It suggests that this typical expression pattern of G1-specific cyclin/CDK complexes could compensate the actions of other cyclins/CDK complexes and allow the progression of cells through the G1 phase continually in endometriotic stromal cells 22B, as proposed in other cell types (72-75). The G1-S transition-specific E2/CDK2 complexes are not down-regulated and this clarifies why progression of cells was not arrested at the G1-S checkpoint in endometriotic stromal cells 22B.

Thus, the combined inhibition of EP2 and EP4 produced additive growth inhibitory effects compared with inhibition of EP2 or EP4 separately, and this growth inhibitory effect was 10% (P<0.01) higher in 12Z cells than that of 22B cells, the effects of pharmacologic inhibition of EP2 and EP4 signaling were in agreement with the effects of genomic ablation of EP2 and EP4 on endometriotic cell proliferation, and the existence of compensatory mechanisms between EP2 and EP4 receptors in mediating PGE₂ signaling in human endometriotic cells, as EP2 and EP4 share common intracellular signaling pathways, and combined inhibition of both EP2 and EP4 is obligatory to understand PGE₂ signaling in proliferation of human endometriotic cells, as described herein (70). Based on this data, the present inventors chose pharmacologic inhibition of both EP2 and EP4 signaling for additional studies considering future therapeutic use of these inhibitors for endometriosis.

Selective inhibition of EP2 and EP4 induces apoptosis of human endometriotic cells: The inventors used the well-characterized human immortalized endometriotic epithelial cells 12Z and stromal cells 22B as a model system to understand PGE₂ signaling in the pathogenesis of endometriosis in women. These endometriotic cells produce large amounts of PGE₂ and inhibition of PGE₂ biosynthesis by selective COX-2 inhibitor induces apoptosis of these cells (11, 38, 39). PGE₂ receptors EP2 and EP4 are abundantly expressed, EP1 is expressed at very low level and EP3 is undetectable (11, 38, 39) in these endometriotic cells. Therefore, we inhibited signaling of EP1, EP2 or EP4 using pharmacological and genomic approaches. The inhibitors used for EP1 (SC19220), EP2 (AH6809) and EP4 (AH23848) competitively bind with the respective EP receptors and inhibit their activations (40-42).

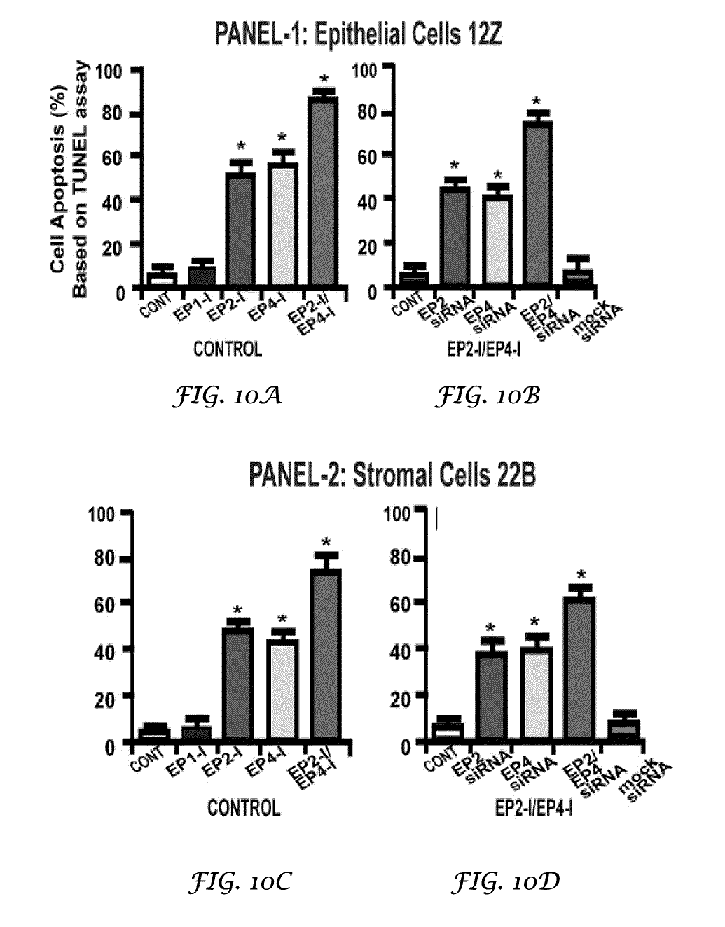

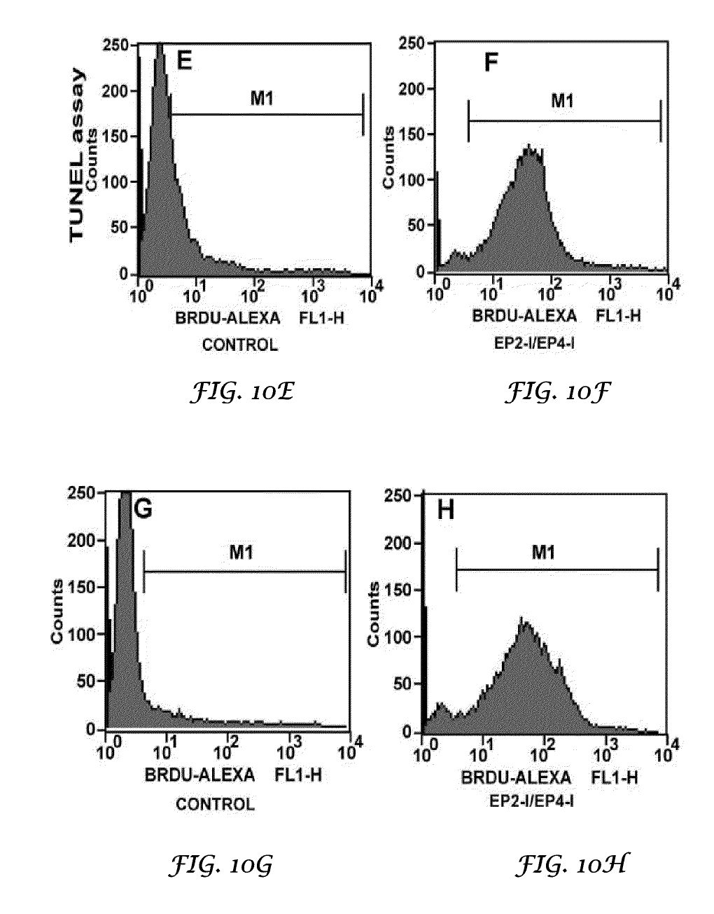

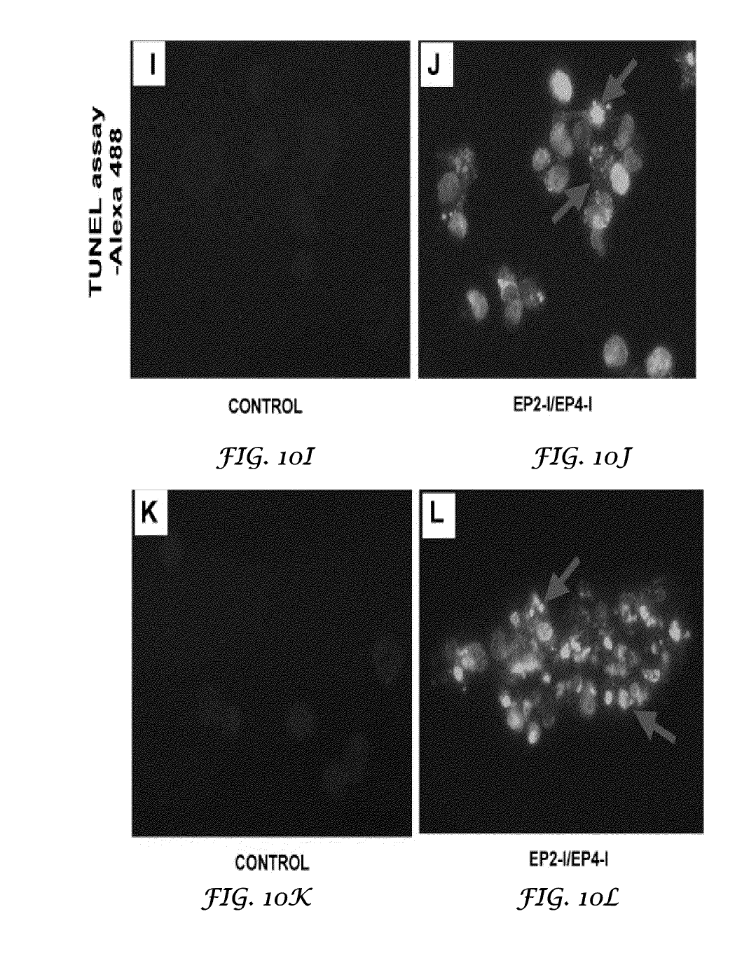

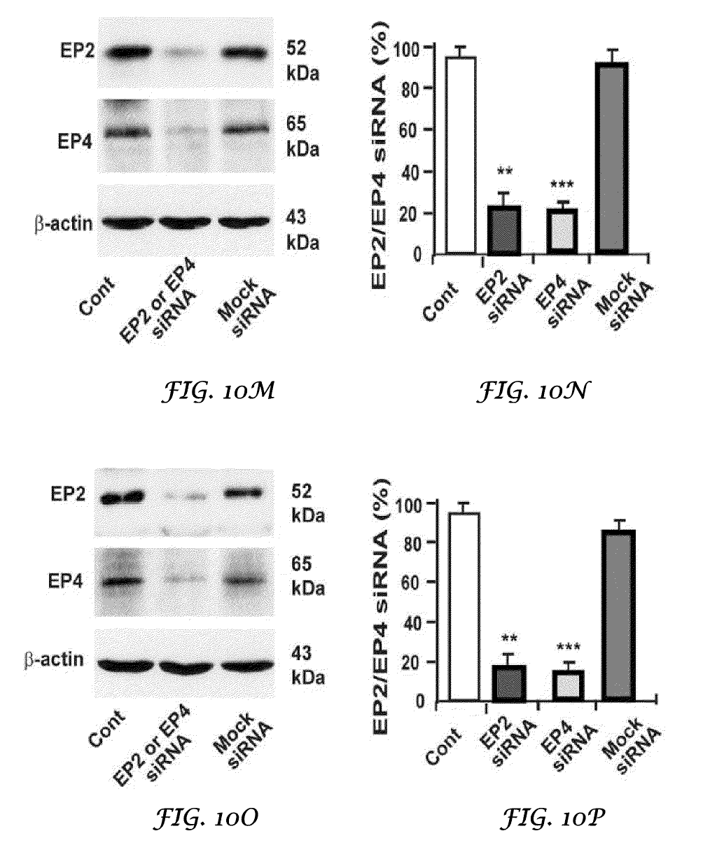

FIGS. 10A-10P of the TUNEL assay using flow cytometry indicated that pharmacological inhibition (FIGS. 10A, 10C, and 10E-10H) of either EP2 or EP4 signaling induced (P<0.05) apoptosis of 12Z and 22B cells ˜55% and ˜45%, respectively. Combined inhibition of EP2 and EP4 produced synergistic effects and induced apoptosis of 12Z and 22B cells ˜85% and ˜75%, respectively. Interestingly, inhibitory effects of EP2 and EP4 on apoptosis were ˜10% higher (P<0.05) in 12Z cells compared to 22B cells. Inhibition of EP1 signaling did not induce apoptosis of 12Z and 22B cells. Consistent with these results, TUNEL assay based on fluorescence microscopy clearly showed that inhibition of EP2 and EP4 signaling induced DNA fragmentation in both 12Z and 22B cells (FIGS. 10I-10L).

The inventors then used siRNA approach to knock-down EP2 and EP4 genes to confirm their roles in the survival of the human endometriotic cells. Gene silencing using SMARTpool siRNA approach resulted in efficient knock-down of EP2 and EP4 genes and resulted in decreased expression of EP2 and EP4 proteins up to 70-80% in both 12Z and 22B cells (FIGS. 10M-10P). Effects of silencing of EP2 or EP4 genes on induction of apoptosis of human 12Z and 22B cells were similar to those involving pharmacological inhibition of EP2 and EP4 signaling. Double knock-down of EP2 and EP4 genes resulted in synergistic effects and induced (P<0.05) apoptosis ˜75% in 12Z cells and 65% in 22B cells (FIGS. 10B and 10D).

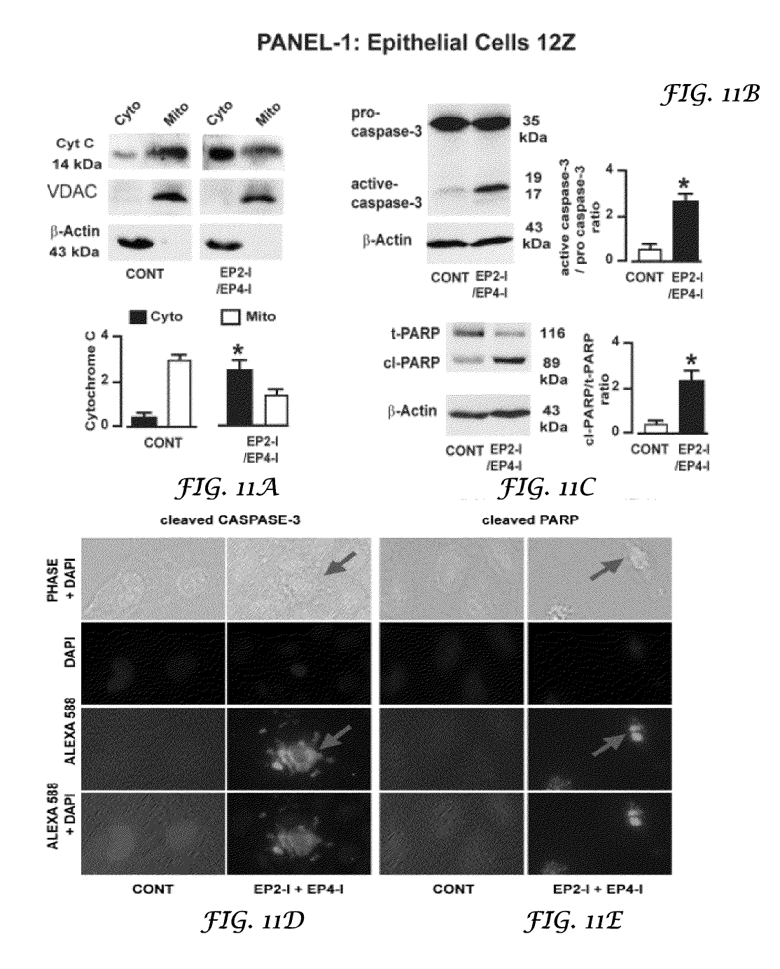

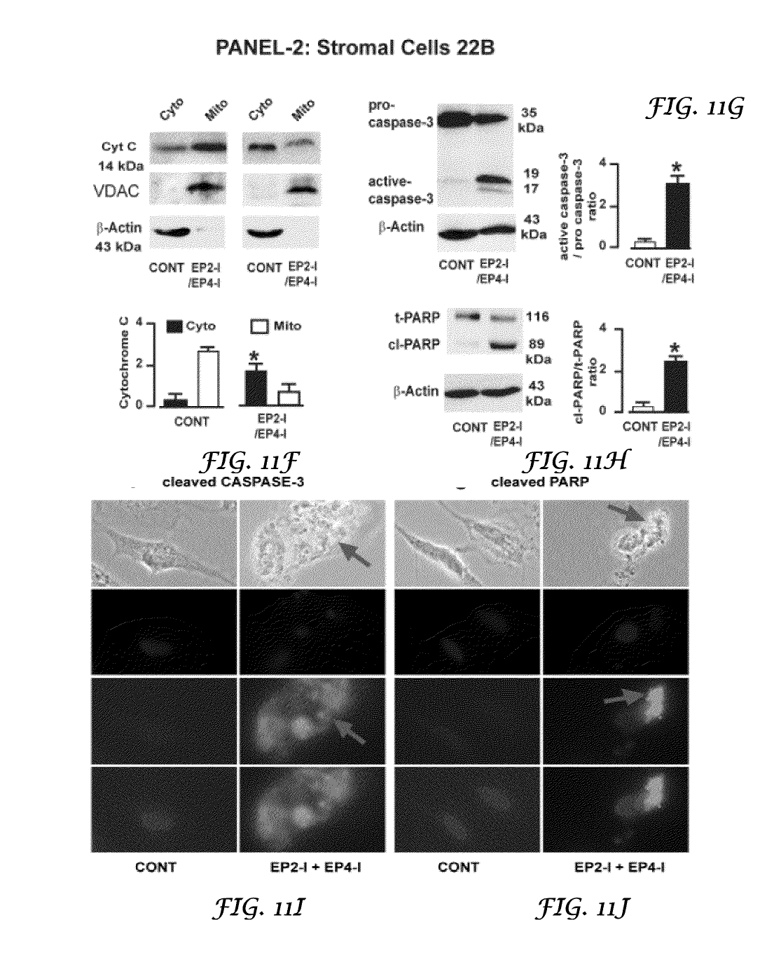

Selective inhibition of EP2 and EP4 releases cytochrome C and activates caspase-3 and PARP in human endometriotic cells: In order to understand the molecular mechanisms by which inhibition of EP2 and EP4-mediated PGE₂ signaling resulted in apoptosis of human endometriotic cells, the inventors determined interactions between PGE₂ signaling and apoptotic machinery. Release of cytochrome C from mitochondria into the cytosol and activation of caspase-3 and nuclear poly (ADP-ribose) polymerase (PARP) enzymes are important terminal events which promote apoptosis of cells (43). Results (FIGS. 11A-11J) indicated that combined inhibition of EP2 and EP4 facilitated release (P<0.05) of cytochrome C from mitochondria into cytosol (FIGS. 11A and 11F), and activated/cleaved (P<0.05) caspase-3 (FIGS. 11B and 11G) and PARP (FIGS. 11C and 11H) proteins in 12Z and 22B cells. Consistent with these results, fluorescence microscopy analyses clearly showed that inhibition of EP2 and EP4 induced activation of caspase-3 (FIGS. 11D and 11I) and PARP (FIGS. 11E and 11J) proteins in 12Z and 22B cells. These results together suggest that selective inhibition of EP2 and EP4 induces apoptosis of human endometriotic cells through cytochrome C/caspase-3/PARP pathways.

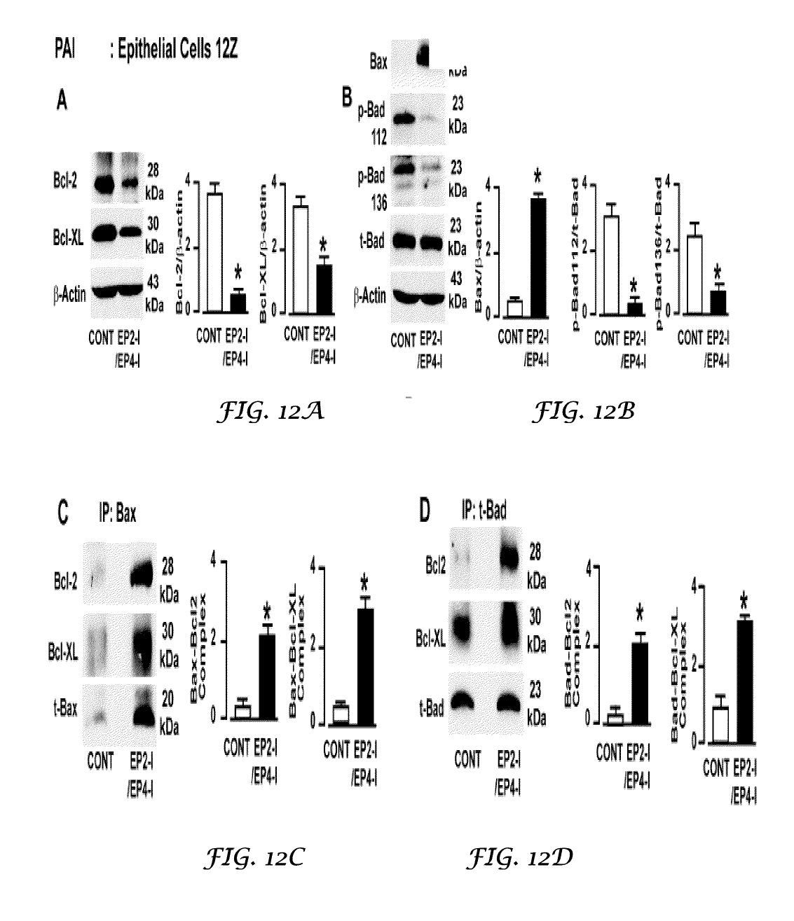

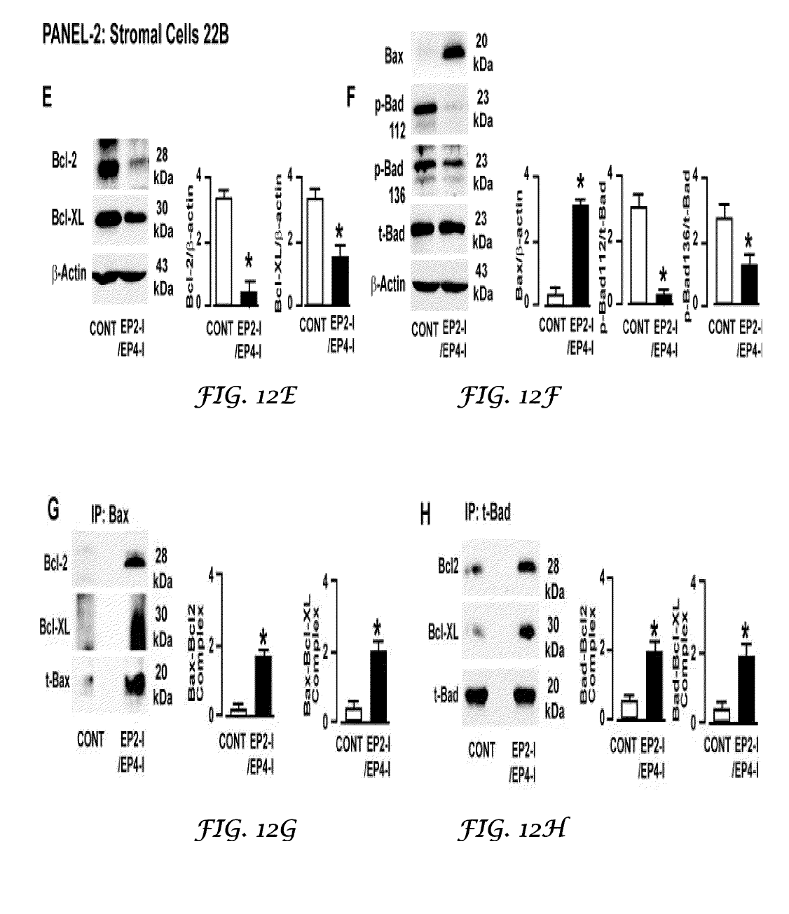

Selective inhibition of EP2 and EP4 augments interactions between anti-apoptotic and pro-apoptotic proteins in human endometriotic cells: The balance between anti-apoptotic proteins (Bcl-2 and Bcl-XL) and pro-apoptotic proteins (Bad and Bax) determines whether cells live or die (44). In the absence of apoptotic stimuli, Bax and Bad protein phosphorylated at serine 112 and 136 bind with 14-3-3 proteins and are sequestered in the cytosol (45). In response to apoptotic stimuli, Bad and/or Bax proteins are dissociated from 14-3-3 proteins, translocate from cytosol to the mitochondrial outer membrane, and dimerize with Bcl-XL and/or Bcl-2 proteins and thereby facilitate the release of cytochrome C (43-45). Therefore, the inventors determined whether inhibition of EP2 and EP4-mediated PGE₂ signaling augments interactions between Bcl-2/Bcl-XL and Bad/Bax proteins in human endometriotic cells. Results (FIGS. 12A-12H) demonstrated that inhibition of EP2 and EP4 decreased (P<0.05) expression of Bcl-2 and Bcl-XL proteins (FIGS. 12A and 12E), increased (P<0.05) expression of Bax protein, and dephosphorylated (P<0.05) Bad protein at serine 112 and 136 sites (FIGS. 12B and 12F) in 12Z and 22B cells. Further, immunoprecipitation results (FIGS. 12C, 12D, 12G, and 12H) indicated that inhibition of EP2 and EP4 increased (P<0.05) interactions between Bax and Bcl-2; Bax and Bcl-XL; Bad and Bcl-2; and Bad and Bcl-XL proteins in 12Z and 22B cells. These results together suggest that selective inhibition of EP2 and EP4 augments interactions between antiapoptotic and proapoptotic proteins and thereby activates intrinsic apoptotic pathways in human endometriotic cells.

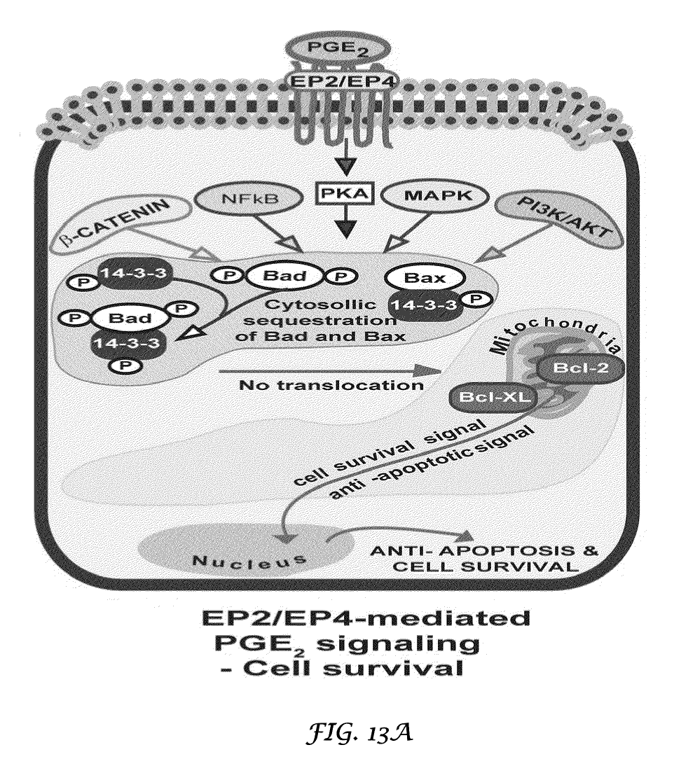

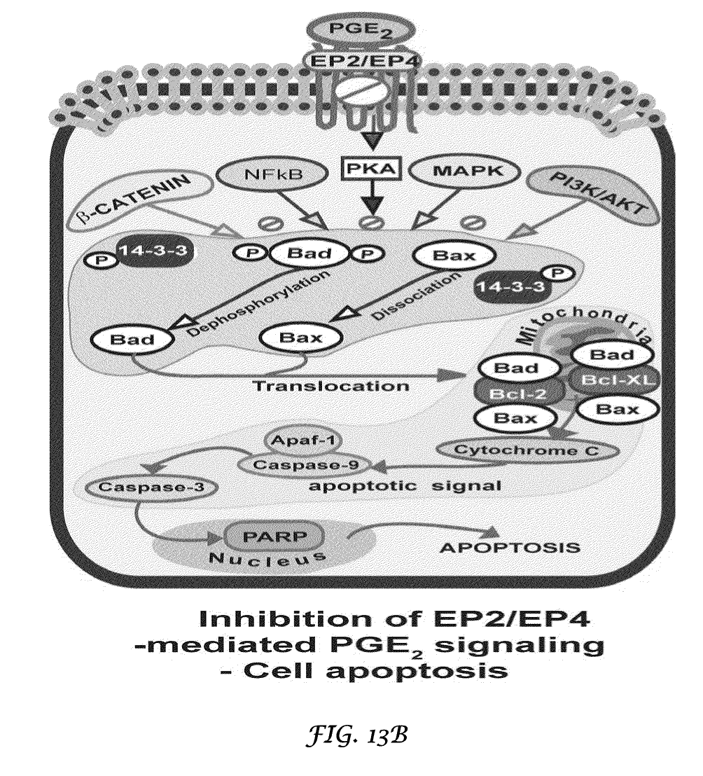

FIGS. 13A and 13B are schematic representations showing that EP2/EP4-mediated PGE₂ signaling leads to cell survival and that the inhibition of EP2/EP4-mediated PGE₂ signaling leads to cell apoptosis, respectively. In FIG. 13A, PGE₂ transactivates ERK1/2, AKT, NFκB, and β-catenin pathways through EP2 and EP4 receptors. Activation of these cell survival pathways phosphorylates Bad protein at serine 112 and 136, sequestrates Bad and Bax proteins in the cytosol with 14-3-3 proteins, and prevents translocation of Bad and Bax from the cytosol to the mitochondria. This prevents interactions between Bax/Bad and Bcl-2/Bcl-XL, and thereby promotes survival of human endometriotic cells. Selective inhibition of EP2 and EP4 impairs ERK1/2, AKT, NFκB, and β-catenin pathways (FIG. 13B). Inhibition of these cell survival pathways results in dephosphorylation of Bad protein at serine 112 and 136, dissociation of Bad and Bax from 14-3-3 proteins, permits translocation of Bad and Bax proteins from the cytosol to the mitochondria, and thereby augments interaction between Bax/Bad and Bcl-2/Bcl-XL proteins. These sequential interactions result in release of cytochrome-C from the mitochondria into the cytosol and activation of caspase-3 and PARP enzymes and eventually culminate in apoptosis of human endometriotic cells

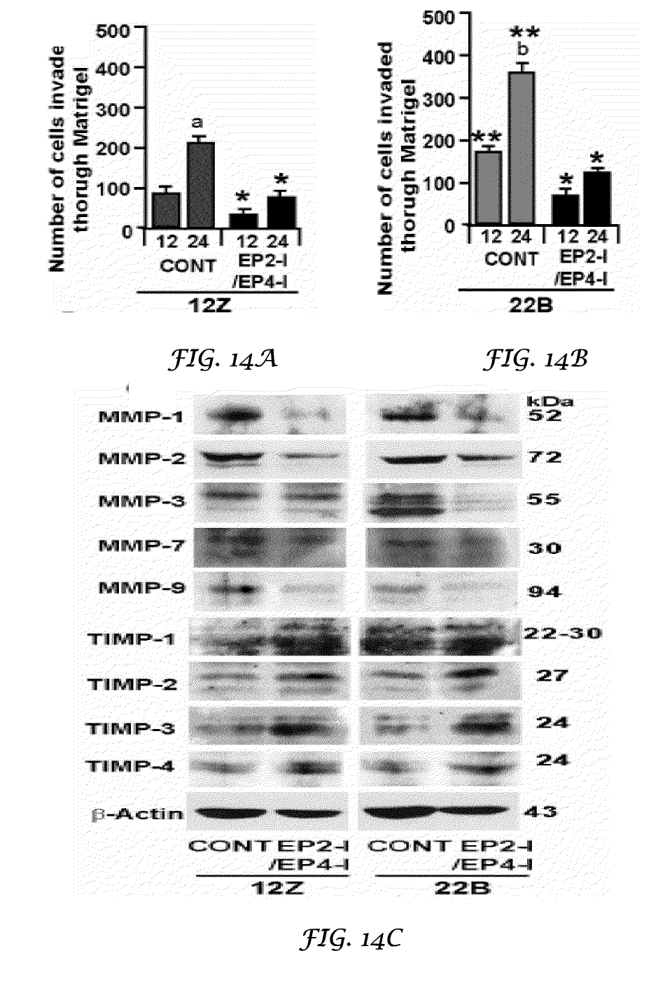

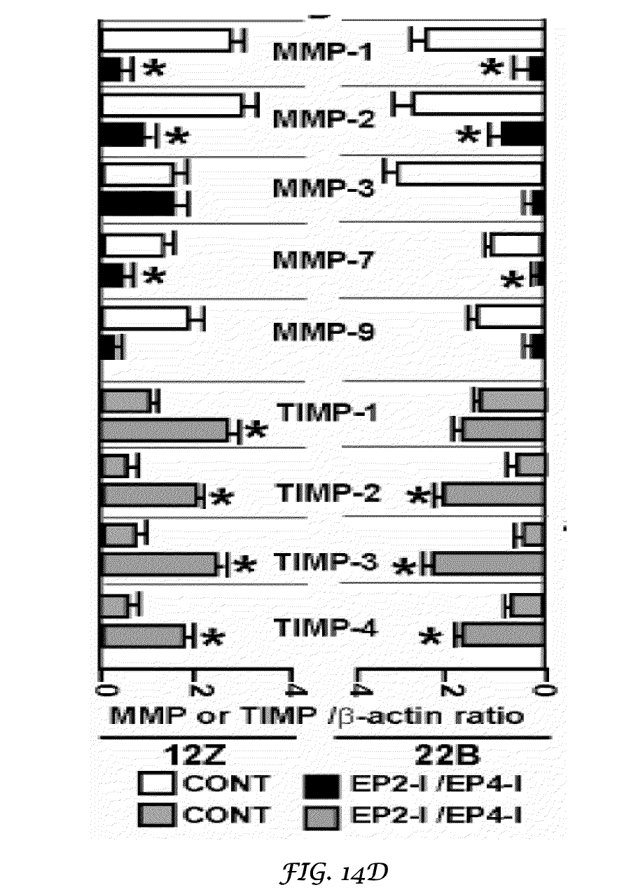

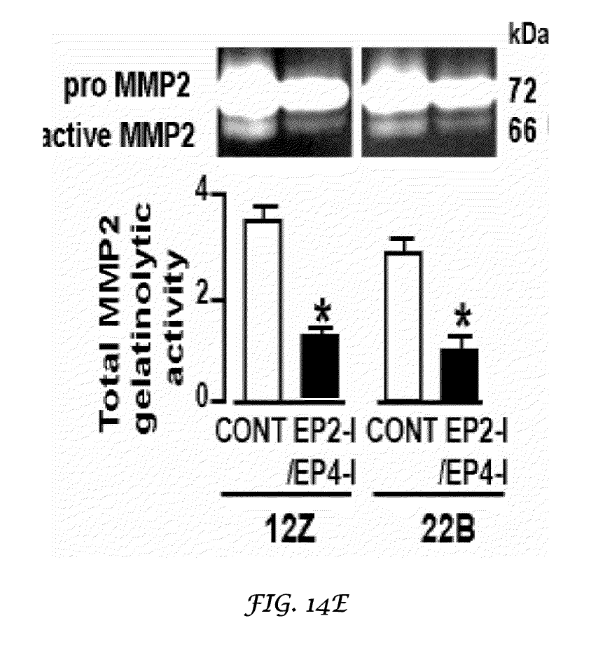

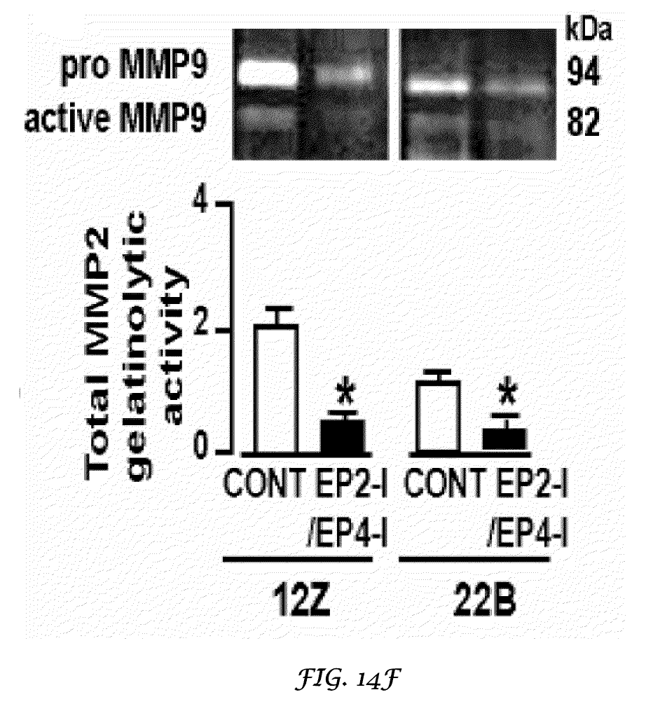

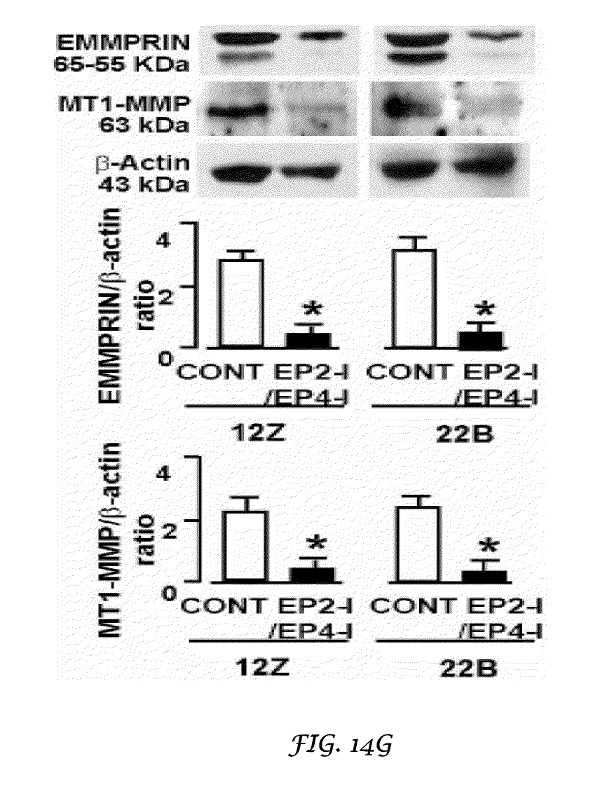

The inventors determined, whether selective inhibition of EP2 and EP4 inhibits invasion of human endometriotic epithelial cells 12Z and stromal cells 22B, and alters expression of MMPs, TIMPs, MT1-MMP and EMMPRIN as previously described. The results (shown in FIGS. 14A-14G) indicated that blockade of EP2 and EP4: (i) inhibited (P<0.05) invasion of 12Z and 22B cells, however; more number of 22B cells invaded (P<0.05) compared to 12Z cells at the given time point (FIGS. 14A and 14B); (ii) decreased expression of MMP1, MMP2, MMP3, MMP1, and MMP9 proteins (FIGS. 14C and 14D); (iv) decreased (P<0.05) activity of MMP2 and MMP9 (FIGS. 14E and 14F); (v) increased (P<0.05) expression of TIMP1, TIMP2, TIMP3, and TIMP4 proteins; and (vi) decreased (P<0.05) expression of MT1-MMP and EMMPRIN proteins in 12Z and 22B in a epithelial-stromal specific manner (FIG. 14G). These results suggest that PGE₂ promotes invasion of endometriotic epithelial and stromal cells through EP2/EP4 and that could be regulated by MMPs-mediated mechanisms.

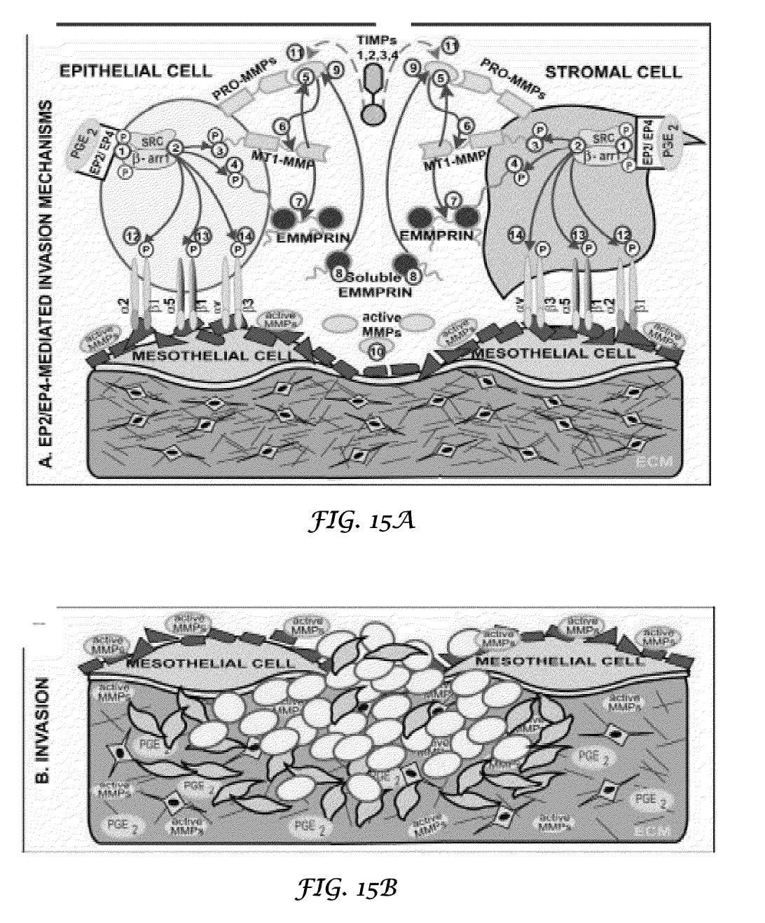

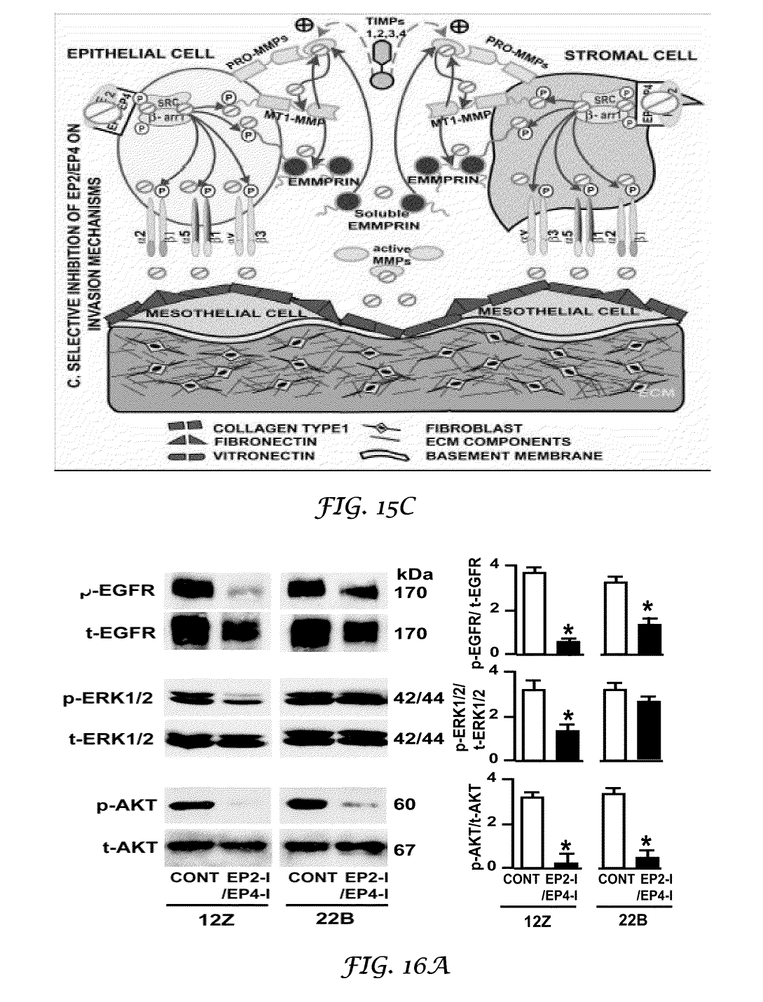

Based on the studies described hereinabove the present inventors present a molecular mechanisms through which selective inhibition of EP2 and EP4 inhibits invasion of human endometriotic cells (FIGS. 15A-15C). As seen in FIG. 15A, (1) PGE2 binds with EP2 and EP4, (2) Activation of EP2/EP4 phosphorylates and activates p-Src416 and p-β-arrestin 1. Activated Src/β-arrestin 1 phosphorylates, (3) MT1-MMP or (4) EMMPRIN by interacting with their cytoplasmic residues and thus results in activation/cleavage of MT1-MMP or EMMPRIN, (5) Activated MT1-MMP interacts with pro-MMPs and converts pro-MMPs into active-MMPs, (6) The active-MMPs in turn activate MT1-MMP and establish positive loop between MT1-MMP and active-MMPs. In addition, this activated MT1-MMP, (7) interacts and cleaves EMMPRIN from cell surface to extracellular/pericellular space and (8) increases soluble EMMPRIN. This soluble EMMPRIN can (9) induce production of active MMPs and (10) increase secretion of active-MMPs into extracellular/pericellular space. Interactions among MT1-MMP, EMMPRIN, and MMPs, (11) preclude or decrease interaction of MMPs with TIMPs in a MMP or TIMP-specific manner. In addition to this, EP2/EP4-mediated signaling can phosphorylate specific integrin receptors for (12) collagen (α2β1), (13) fibronectin (α5β1), and (14) vitronectin (αvβ3) through Src/β-arrestin-1 by interacting with cytoplasmic tail of β subunits of the integrin receptors and thus promote adhesion of endometriotic epithelial and stromal cells prior to invasion.

In FIG. 15B activation of EP2/EP4-mediated PGE₂ signaling and positive interactions among MT1-MMP, EMMRPIN, and active MMPs consistently degrades ECM and promotes invasion of endometriotic epithelial and stromal cell into peritoneum. In FIG. 15C selective inhibition of EP2 and EP4 inhibits phosphorylation of Src416 and β-arrestin 1 and thus decreases the positive interactions and feed-back loop among MT1-MMP, EMMPRIN and MMPs and increases the negative interactions between MMPs and TIMPs and thus inhibits invasion of human endometriotic epithelial and stromal cells into peritoneum.

PGE₂ signaling network: PGE₂ transactivates EGFR signaling through a c-Src/β-arrestin 1 complex which in turn activates ERK1/2 and PI3K-AKT pathways (25, 27, 28, 31), activates Wnt/β-catenin signaling pathways through binding with regulator of G protein signaling (RGS) domain of axin and AKT-mediated phosphorylation/inactivation of GSK3β (29, 30), and interacts with NFκB pathways (25) in malignant tumor cells. EGFR signaling phosphorylates Bad protein through ERK1/2 and AKT pathways (46, 47). NFκB signaling increases expression of antiapoptotic proteins Bcl2 and Bcl-XL (48). The β-catenin pathway interacts with proapoptotic and antiapoptotic proteins directly or indirectly (49). Therefore, we sought to determine interactions between PGE₂ signaling and EGFR, NFκB and β-catenin pathways in order to understand how these survival pathways are compromised to facilitate apoptosis in human endometriotic cells.

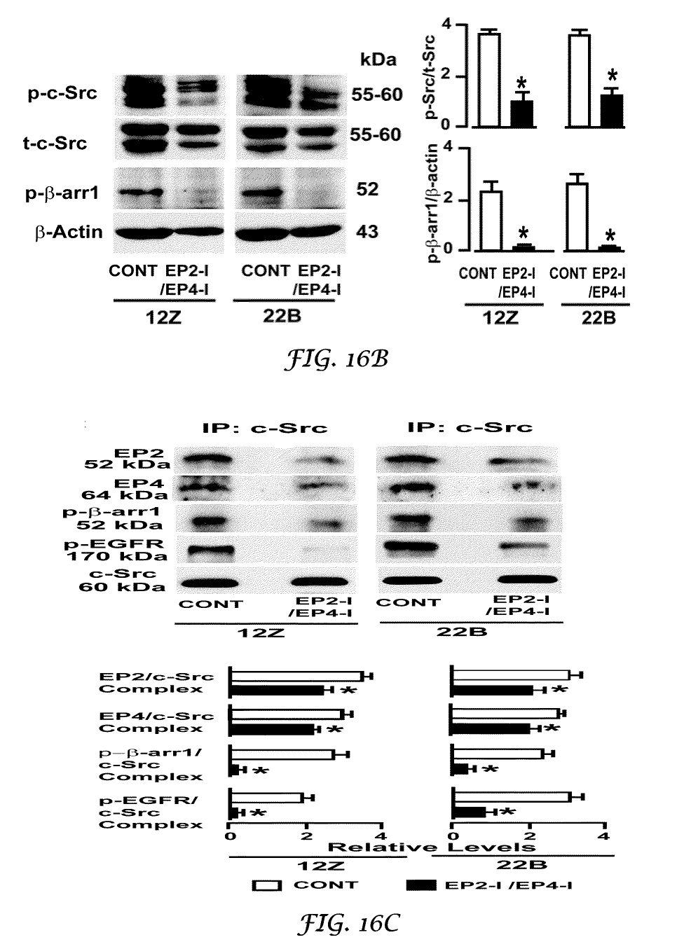

First, the inventors examined whether selective inhibition of EP2/EP4 affects EGFR signaling pathways in human endometriotic cells. It is well known that activation/phosphorylation of EGFR in turn triggers Ras-Raf-ERK1/2 and PI3K-AKT signaling modules, and activation of EGFR protein can be inhibited either by dephosphorylation and/or by down-regulation (50). Results (FIG. 16A) indicated that inhibition of EP2 and EP4 dephosphorylated and down regulated (P<0.05) EGFR protein in 12Z and 22B cells. ERK1/2 protein was dephosphorylated (P<0.05) in 12Z cells but not in 22B cells. Interestingly, AKT protein was dephosphorylated (P<0.05) in both 12Z and 22B cells. In order to determine whether this cross-talk between EP2/EP4 and EGFR is mediated through c-Src and β-arrestin 1, the inventors studied their expression levels. Results (FIG. 16B) demonstrated that inhibition of EP2 and EP4 dephosphorylated (P<0.05) c-Src and β-arrestin 1 proteins in 12Z and 22B cells. Next, the inventors determined interactions among EP2, EP4, c-Src and β-arrestin 1, and EGFR in order to gain further mechanistic insights. Results (FIG. 16C) indicated that inhibition of EP2 and EP4 decreased (P<0.05) interactions among EP2/EP4, c-Src, β-arrestin 1 and EGFR proteins in 12Z and 22B cells. Taken together, these results suggest that PGE₂ transactivates EGFR through EP2 and EP4 receptors, and this signaling is transmitted intracellularly through a c-Src/β-arrestin 1 complex in human endometriotic cells.

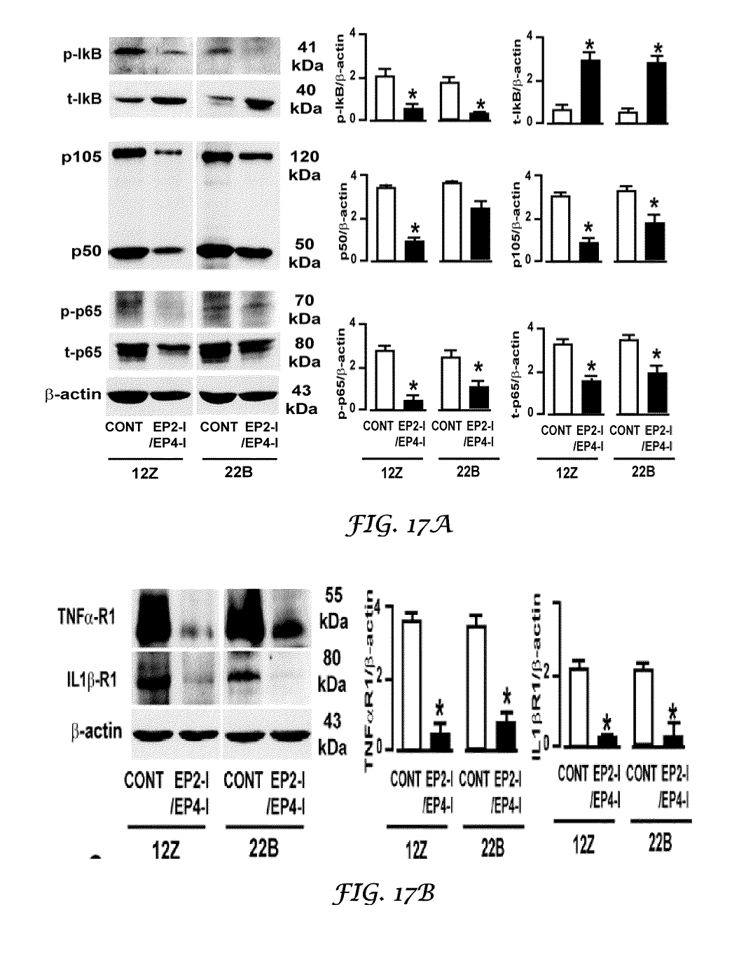

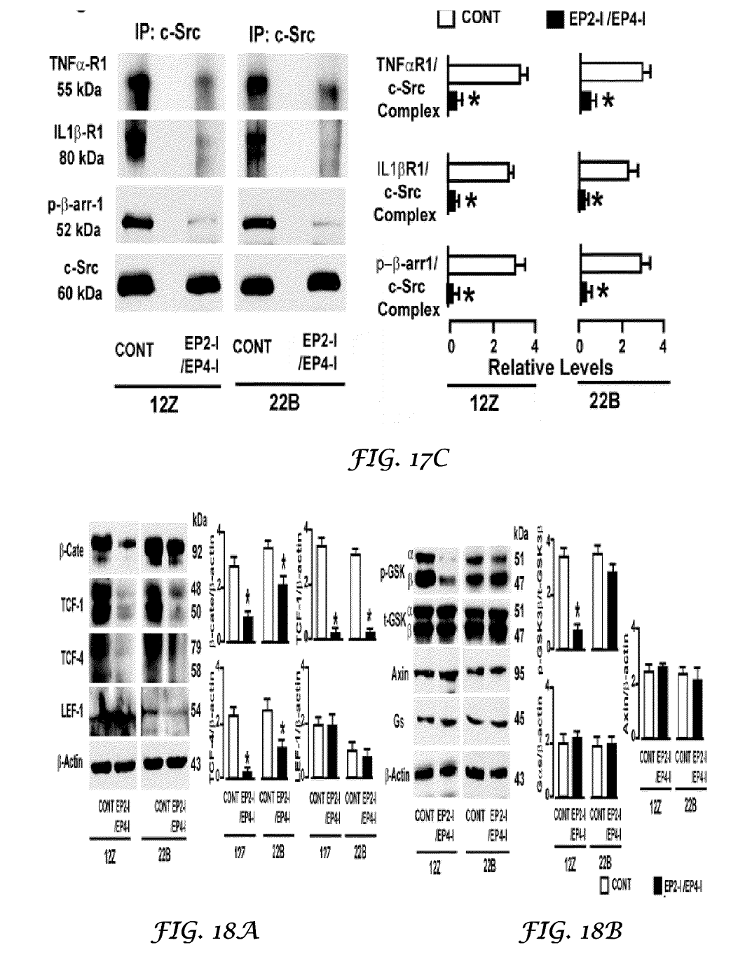

Secondly the inventors, examined whether selective inhibition of EP2/EP4-mediated PGE₂ signaling affects NFκB signaling cascades in human endometriotic cells. Heterodimer complex of p50/p65 is the most common active form of NFκB in the majority of cells. In the absence of NFκB stimuli, p50 and p65 proteins are sequestered in the cytoplasm with their inhibitory protein IκBα and form an inactive protein complex, p50/p65/IκB. In response to cytokines TNFα, IL1-β or other stimuli, IκBα protein is phosphorylated and targeted for degradation and allow formation of active p50/p65 heterodimer and translocation into the nucleus (48). Results (FIG. 17A) indicated that inhibition of EP2 and EP4: (i) dephosphorylated (P<0.05) IκB_{α}; (ii) decreased (P<0.05) phosphorylation and abundance of p65 protein; and decreased (P<0.05) abundance of precursor p105 in 12Z and 22B cells, while, it decreased (P<0.05) abundance of active p50 proteins only in 12Z cells but not in 22B cells. Next, the inventors determined whether this NFκB signaling complex is dependent or independent of TNFαR1 and/or IL1βR1 involvement. Results (FIG. 17B) demonstrated that inhibition of EP2 and EP4 decreased (P<0.05) expression of TNFαR1 and IL1βR1 in 12Z and 22B cells. This led us to explore interactions between EP2/EP4 and TNFαR1 or IL1βR1 to derive further mechanistic insights. The inventors conclude that c-Src/β-arrestin-1 protein complex mediates cross-talk between EP2/EP4 and TNFαR1 or IL1βR1 in 12Z and 22B cells, because this protein complex is involved in mediating cross-talk between EP2/EP4 and EGFR in these cells. In support of this notion, immunoprecipitation results (FIG. 17C) clearly demonstrated that inhibition of EP2 and EP4 decreased (P<0.05) interaction between c-Src/β-arrestin-1 complex and TNFαR1 or IL1βR1 in 12Z and 22B cells. These results together suggest that PGE₂ activates NFκB pathways through EP2 and EP4 receptors by transactivating TNFαR1 and IL1βR1, and this signaling is routed intracellularly through a c-Src/β-arrestin 1 complex in human endometriotic cells. Further, selective inhibition of EP2 and EP4-mediated PGE₂ signaling disturbs NFκB signaling pathways at multiple levels, that is, at processing of active p50, dephosphorylation of p65, and dephosphorylation and stabilization of IκBα proteins in a cell-specific manner. It appears that stabilization of IκBα is the key step which could result in re-establishment of inactive complex p50/p65/IκB and affect NFκB down-stream signal in both 12Z and 22B cells.

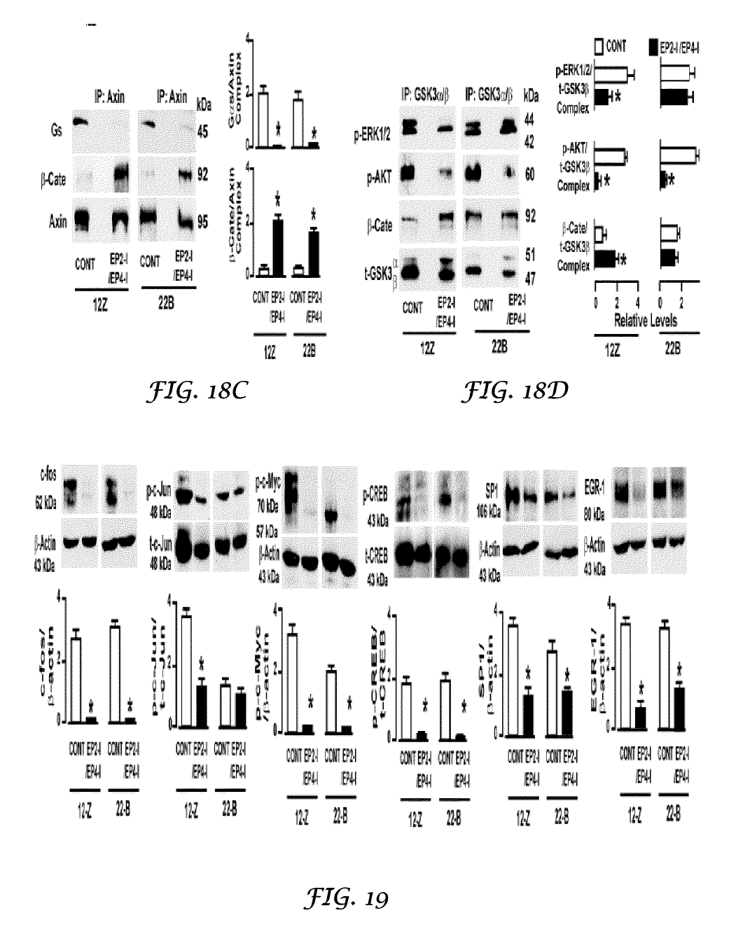

Thirdly, the inventors examined whether selective inhibition of EP2/EP4-mediated PGE₂ signaling affects β-catenin pathways in human endometriotic cells. In the absence of Wnt/β-catenin or other stimuli, β-catenin is sequestered in the cytosol by a destruction complex consisting of glycogen synthase kinase 3β (GSK3β, axin, and adenomatosis polyposis coli (APC) and targeted for degradation. In response to Wnt/β-catenin stimuli, β-catenin is released from this destruction complex, translocates into nucleus and forms a transcriptionally active complex with Tcf/Lef family of transcription factors (49). Results (FIG. 18A) indicated that inhibition of EP2 and EP4 resulted in degradation (P<0.05) of active β-catenin in 12Z and 22B cells, decreased (P<0.05) expression of β-catenin binding transcriptional factors TCF-1 and TCF-4 in 12Z and 22B cells, and did not decrease LEF-1 in both cells. Next, the inventors examined whether this degradation of β-catenin is due to changes in destruction complex proteins GSK3β, axin and Gs. Results (FIG. 18B) indicated that inhibition of EP2 and EP4 dephosphorylated (P<0.05) GSK3β in 12Z cells and but not in 22B cells, and did not affect relative expression of either Gs or axin proteins in 12Z and 22B cells. Then, the inventors examined the mechanisms by which inhibition of EP2 and EP4-mediated PGE₂ signaling led to destruction of β-catenin. First, we examined the Gs/axin/β-catenin axis. Results (FIG. 18C) indicated that inhibition of EP2 and EP4 decreased (P<0.05) interactions between axin and Gs and increased association between axin and β-catenin in 12Z and 22B cells. Second, the inventors examined GSK3β/β-catenin axis. Results (FIG. 18D) indicated that inhibition of EP2 and EP4 increased interactions between β-catenin and GSK3β in 12Z but not in 22B cells. Next, the basis for cell-specific differences of EP2 and EP4-mediated PGE₂ signaling on regulation of GSK3β in 12Z and 22B cells was analyzed. The inventors concluded that one of the constructively active signaling pathways might contribute to this cell-specific regulation of GSK3β in 22B cells. Previous studies have reported that GSK3β is regulated by AKT and ERK1/2 pathways in cancer cells (30, 51). The results shown in FIG. 8 indicated that in response to inhibition of EP2 and EP4, AKT but not the ERK1/2 pathway was inhibited in 22B compared to 12Z cells; therefore, we examined interactions among GSK3β, ERK1/2 and AKT. In support of this notion, immunoprecipitation results (FIG. 18D) demonstrated that inhibition EP2 and EP4 decreased (P<0.05) interactions between GSK3β and AKT and/or ERK1/2 in 12Z cells. While in 22B cells, inhibition EP2 and EP4 decreased (P<0.05) interactions between GSK3β and AKT but failed to decrease interaction between GSK3β and ERK1/2. These results together suggest that selective inhibition of EP2 and EP4 degrades β-catenin in a cell-specific manner by impairing Gs/axin/β-catenin, ERK1/2/GSK3β/β-catenin, and AKT/GSK3β/β-catenin signaling modules in 12Z cells, and Gs/axin/β-catenin and AKT/GSK3β/β-catenin signaling modules in 22B cells.

Finally, the inventors determined whether inhibition of EP2 and EP4 affects the important transcriptional factors associated with EGFR, AKT, ERK1/2, NFκB and β-catenin signaling pathways in human endometriotic cells. Results (FIG. 19) indicated that inhibition of EP2 and EP4 decreased (P<0.05) expression of c-fos, SP1, EGR-1 and phosphorylation c-myc and CRBE proteins in 12Z and 22B cells. In contrast, decreased phosphorylation of c-Jun protein was observed in 12Z cells but not in 22B cells. These results suggest that EP2 and EP4-mediated PGE₂ signaling regulate expression and activation of multiple transcriptional factors in human endometriotic cells.

Expression of PGE₂ signaling components in endometriotic tissues in human: To conclude, the inventors examined the relative expression of major PGE₂ signaling components in endometriosis and endometrial tissues from women. Results (FIGS. 20A-20U) indicated that Bcl2, Bcl-XL, p-Bad112, p-Bad136, p-EGFR, p-ERK1/2, p-AKT, p-IκB, and active β-catenin proteins were more abundantly expressed in endometriosis tissues during proliferative phase of the menstrual cycle. Interestingly, the spatial relative expression of these proteins in glandular epithelium and stroma were higher (P<0.05) in endometriosis (ectopic endometrium) compared to healthy endometrial tissues (eutopic endometrium). These results suggest that PGE₂ signaling components associated with antiapoptosis/prosurvival pathways are abundantly expressed in endometriosis tissues in women.

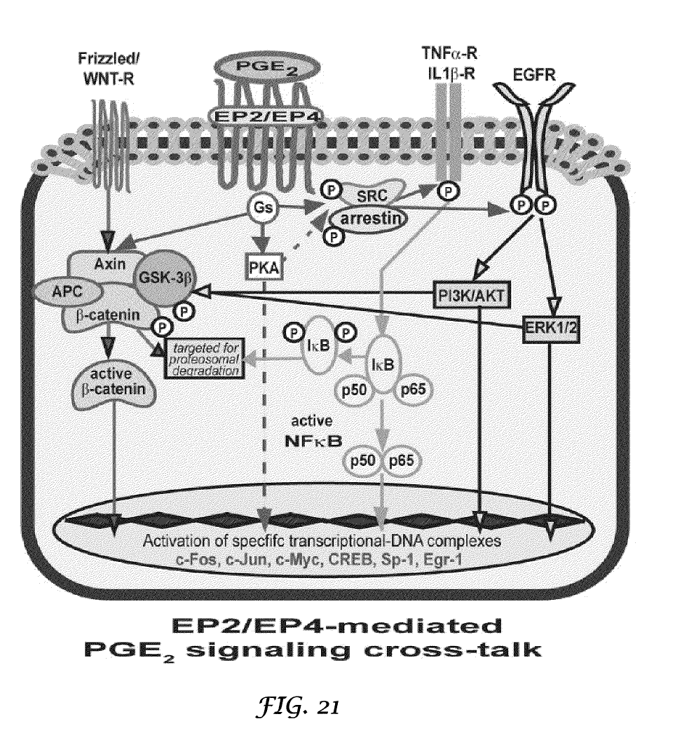

Involvement of prostaglandins in the pathogenesis of endometriosis has been previously reported; however, the underlying mechanisms are largely unknown. Analysis of PGE₂-induced protection of human endometriotic cells from apoptosis has revealed a complex organization of signaling pathways. The results obtained by the studies in the present invention strongly suggest that ability of human endometriotic cells to circumvent apoptosis signals is associated with increased PGE₂ signaling, abundant expression of Bcl2 and Bcl-XL proteins, low expression of Bax protein, phosphorylation/inactivation of Bad protein, and activation of multiple cell survival signaling pathways. Based on the results of the present study, the inventors molecular mechanisms by which PGE₂ promotes survival of human endometriotic cells as illustrated in FIG. 21. PGE₂ regulates these complex molecular interactions and promotes survival of human endometriotic cells through EP2 and EP4 receptors by transactivating multiple complex signaling modules c-Src/β-arrestin 1/EGFR/ERK1/2 or AKT, c-Src/β-arrestin 1/TNFαR1 and/or IL1βR1/IκB/NFκB, Gs_{α}/axin/β-catenin, ERK1/2/GSK3β/β-catenin and AKT/GSK3β/β-catenin. Selective inhibition of EP2 and EP4 impairs these cell survival pathways and augments interactions between proapoptotic proteins (Bax/Bad) and antiapoptotic proteins (Bcl-2/Bcl-XL), facilitates the release of cytochrome C, and thus activates caspase-3/PARP pathways.

Furthermore, our data suggest that activity of CDKs is inhibited by their positive regulatory subunit cyclins as the major mechanism but not through negative regulatory subunit CDK inhibitors, as described in other cells in response to inhibition of COX-2 (63). The CDK inhibitors p15, p16, p21, and p27 are abundantly expressed in 12Z and 22B cells at basal conditions. It is well known that increased expression of CDK inhibitors induces post-translational inhibition of CDK activity in numerous cancer cell types under various conditions (72-75). Surprisingly, selective inhibition of EP2/EP4 did not increase expression of CDK inhibitors p15, p16, p21, and p2′7, in contrast decreased their expression levels. The available evidence in cancer cells indicate that CDK inhibitors, for example p21, are regulated by p53-dependent and p53-independent mechanisms (76-77). Increased expression of p21 inhibits p53-induced apoptosis of cancer cells, whereas suppression of p21 expression shifts cells from cell cycle arrest to apoptosis pathways (76-77). Based on results (76-77), the present inventors hypothesize that decreased expression of CDK inhibitors in response to selective inhibition of EP2 and EP4 in human endometriotic epithelial cells 12Z and stromal cells 22B would have favored the apoptosis (70) more than cell cycle regulation.

Selective inhibition of EP2 and EP4 decreases cell proliferation to a greater degree in the endometriotic epithelial cells 12Z than in stromal cells 22B. Cell cycle arrest at both G1-S and G2-M checkpoints in 12Z cells compared with only at the G2-M checkpoint in 22B cells could be responsible for these epithelial-stromal cell specific growth inhibitory effects. We have recently found that phosphorylation of ERK1/2 and NFkB, dephosphorylation of GSK3b, stability of β-catenin, and activation of c-jun are not completely suppressed by inhibition of EP2 and EP4 in 22B compared with 12Z cells (70). It suggests that epithelial-stromal-specific suppression of mitogenic/survival pathways ERK1/2, NFkB or β-catenin might be one of the contributing factors for decreased proliferation of 12Z compared with 22B cells.

In addition, the results of studies described herein indicate that inhibition of EP2 and EP4 did not prevent proliferation of normal HES and HESC cells. In contrast, inhibition of EP2 and EP4 decreases proliferation of endometriotic epithelial cells 12Z and stromal cells 22B. We found that HES and HESC do not express COX-2 and produce PGE₂; in contrast, 12Z and 22B cells express COX-2 abundantly and consistently and produce large amounts of PGE₂ endogenously at basal condition. Based on the present and previous findings (11) the inventors hypothesize that inhibition of EP2/EP4 would produce significant antiproliferative effects in cells that depend on COX-2, PGE₂, and EP2/EP4-mediated signaling for their proliferation and survival. The inventors presume that magnitude and intensity of COX-2-derived PGE₂ production and timely EP2- and EP4-mediated PGE₂ signaling greatly differs between eutopic endometria (physiological state) and ectopic endometrial (pathological state). This notion is supported by the recent findings that PGE₂ stimulates steroidogenic genes in ectopic endometriotic cells; in contrast it did not stimulate steroidogenic genes in eutopic endometrial cells from endometriosis-free women (78). The COX-2 (11-13), EP2, and EP4 (70) proteins are more abundantly and constitutively expressed in ectopic endometria, but temporally regulated in eutopic endometria from women without endometriosis during the menstrual cycle (53, 79-80).

Data presented in the present invention indicates that activation of EP2/EP4 receptors by PGE₂ in turn activates Src/β-arrestin 1 complex, which is a step in EP2/EP4 signal transduction. Followed by, Src/β-arrestin 1 complex could mediate transactivation between EP2/EP4 and EGFR or TNFα/ILβ receptors. At present, there is no data to indicate which of these receptors is activated first, however; the inventors indicate that EGFR could be activated earlier than TNFα/ILβ receptors. Second, activation of EGFR and TNFα/ILβ receptors could result in activation/suppression of multiple down-stream cell signaling pathways. Third, activated AKT and ERK could establish activation of β-catenin. Thus, PGE₂ could integrate EGFR, NFκB, and β-catenin pathways intracellularly that in turn could activate or inactivate transcription of genes, translation of proteins, or protein-protein interactions in 12Z and 22B cells

The remarkable redundancy of signaling pathways that control interactions between proapoptotic and antiapoptotic proteins argues for the importance of inhibiting multiple pathways to prevent the survival of human endometriotic cells. Relative expressions of proteins involved in PGE₂ signaling including EP2, EP4, p-Bad112, p-Bad136, Bcl2, Bcl-XL, p-ERK1/2, p-AKT, p-IκB and β-catenin are significantly higher in ectopic endometriotic tissues compared to eutopic endometrial tissues in women in vivo, suggesting ERK1/2, AKT, NFκB or β-catenin pathways are highly activated in endometriosis. Therefore, inhibition of each of these pathways is possible using selective inhibitors. However, the limitations of inhibiting a single pathway are: (i) possible compensation by other linear survival pathways resulting in rescue of endometriotic cells from apoptosis; and (ii) complete inhibition of a single pathway by selective inhibitors may block/abolish the particular pathway required for normal reproductive, physiological, developmental and homeostasis processes. The most exciting aspect of the present invention is the selective inhibition of EP2 and EP4, which only partially suppresses but does not abolish multiple signaling pathways including ERK1/2, AKT, NFκB and β-catenin pathways. Further, signal transduction pathways that promote cell survival are compromised while pathways that promote cell apoptosis are augmented. Taken together, the data of the present invention suggests that selective inhibition of EP2 and EP4 could potentially suppress the adverse effects of upregulated ERK1/2, AKT, NFκB and β-catenin pathways in the pathogenesis of endometriosis and could preserve the beneficial role of these important pathways in normal endometrial cell functions. Therefore, selective inhibition of EP2 and EP4 receptors advantages over inhibition of ERK1/2, AKT, NFκB or β-catenin pathways separately.

Interestingly, inhibition of EP2 and EP4 induces apoptosis to a greater degree in the endometriotic epithelial cells 12Z compared to stromal cells 22B. This cell-specific effect is similar to that of selective inhibition of COX-2 (11) in these cells. It appears that difference in the level of suppression of ERK1/2, NFκB or β-catenin pathways might be one of the reasons for increased survival of 22B compared to 12Z cells. In support of this notion, phosphorylation of ERK1/2, NFκB, phosphorylation of GSK3β, stability of β-catenin and activation of c-jun are not completely suppressed by inhibition of EP2 and EP4 in 22B compared to 12Z cells. Microscopically, endometriosis lesions are characterized by the presence of endometriosis glands lined with cylindrical and flattened epithelial cells and surrounded by dense stromal cells. Variations in the growth patterns of endometriosis lesions are considered to be regulated by epithelial-stromal interactions (8, 52). One of the most interesting aspects of the present invention is that selective inhibition of EP2 and EP4 inhibits survival of epithelial cells by 85% and stromal cells by 75%, suggesting the inhibition of epithelial-stromal interactions and formation of endometriosis glands.

Expression of EP2 mRNA in endometrial biopsies was not modulated across the menstrual cycle; however, EP4 mRNA expression was significantly higher in proliferative biopsies than in secretory phase samples (53). Since EP2/EP4 receptors are expressed in endometrium during the menstrual cycle in women, these receptors would involve in mechanisms associated with menstruation, differentiation of endometrium after menstruation, and establishment of pregnancy. Whether pharmacological inhibition of EP2/EP4 receptors could affect these processes in women is not known. Interestingly, data from EP2 and EP4 knockout mice indicated that EP4 gene knockout mice were fertile (54) and EP2 gene knockout mice had normal ovulation and implantation but suffered from a fertilization defect which was overcome by in vitro fertilization (55). These data might not necessarily mirror the human condition because mice are non menstruating animals. Baboon could be the ideal preclinical model system to evaluate the effects of EP2/EP4 inhibitors on menstruation and pregnancy. Further, the inventors indicate that if combined inhibition of EP2/EP4 affects menstruation and pregnancy in women, it could be possible to overcome or minimize this effect by blocking EP2 or EP4 independently in order to get 35-40% growth arrest of endometriotic cells. This would allow compensatory mechanisms between these two receptors to mediate the physiological functions and thus would allow menstruation and successful pregnancy in endometriosis women.

The advantages of selective inhibition of EP2 and EP4 include but are not limited to: (i) inhibition of growth and survival of endometriotic cells; (ii) decreased PGE₂-induced inflammation and pain; (iii) a permissive action on menstruation, ovulation and pregnancy; and (iv) the absence of a hypoestrogenic state or temporary menopause in reproductive age women resulting in improved reproductive health. These are the goals for endometriosis therapies envisioned for several decades. Based on the results of the present invention, the inventors suggest that inhibition of EP2/EP4 receptors could emerge as a potential therapy for treatment of endometriosis, preferably stages I and II (active phase of disease characterized by red peritoneal lesions).

In conclusion, the results of the present invention for the first time indicate that PGE₂ promotes survival of human endometriotic cells through EP2 and EP4 by activating multiple cell survival signaling pathways. Selective and combined inhibition of EP2 and EP4 impairs these survival pathways and activates intrinsic apoptotic pathways thereby induces apoptosis of human endometriotic cells, inhibits proliferation of human endometriotic epithelial cells 12Z through G1-S and G2-M and stromal cells 22B through G2-M cell cycle arrest through regulation of specific cyclins and CDKs. The present invention provide a direct molecular link between PGE₂ signaling and growth and survival of human endometriotic cells Inhibition of EP2 and EP4-mediated signaling could emerge as a potential non-estrogen targeted therapy for the treatment of endometriosis in women. Results of the present invention suggest potential opportunities for translational studies that could lead to preclinical and clinical-phase trials.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

US Patent Publication No. 2009060997: Process for producing a pharmaceutical preparation for therapeutic treatment of endometriosis containing a combination of a gestagen and (6s)-5-methyltetrahydrofolate.
Publication No. WO2006116873: Diagnosis and treatment of endometriosis.
Publication No. 2008058514: Preparations comprising Boswellic acids for inhibiting the synthesis of prostaglandin E2.
U.S. Pat. No. 5,744,464: Antigestagens for the inhibition of uterine synthesis of prostaglandin.
1. Giudice L C, Kao L C 2004 Endometriosis. Lancet 364:1789-1799.
2. Olive D L 2008 Gonadotropin-releasing hormone agonists for endometriosis. N Engl J Med 359:1136-1142.
3. Sampson J 1927 Peritoneal endometritis due to menstrual dissemination of endometrial tissue into the peritoneal cavity. Am J Obstet Gynecol 14:442-469
4. Bulun S E, Yang S, Fang Z, Gurates B, Tamura M, Sebastian S 2002 Estrogen production and metabolism in endometriosis. Ann N Y Acad Sci 955:75-85; discussion 86-78, 396-406.
5. Hastings J M, Fazleabas A T 2003 Future directions in endometriosis research. Semin Reprod Med 21:255-262.
6. Osteen K G, Bruner-Tran K L, Eisenberg E 2005 Endometrial biology and the etiology of endometriosis. Feral Steril 84:33-34; discussion 38-39.
7. Guo S W, Olive D L 2007 Two unsuccessful clinical trials on endometriosis and a few lessons learned. Gynecol Obstet Invest 64:24-35.
8. Banu S K, Starzinski-Powitz A, Speights V O, Burghardt R C, Arosh J A 2009 Induction of peritoneal endometriosis in nude mice using human immortalized endometriosis epithelial and stromal cells: A potential experimental tool to study molecular pathogenesis of endometriosis in human. Fertil Steril 91: 2199-2209.
9. Wu M H, Shoji Y, Chuang P C, Tsai S J 2007 Endometriosis: disease pathophysiology and the role of prostaglandins. Expert Rev Mol Med 9:1-20.
10. Laschke M W, Elitzsch A, Scheuer C, Vollmar B, Menger M D 2007 Selective cyclo-oxygenase-2 inhibition induces regression of autologous endometrial grafts by down-regulation of vascular endothelial growth factor-mediated angiogenesis and stimulation of caspase-3-dependent apoptosis. Fertil Steril 87:163-171.
11. Banu S K, Lee J, Speights V O, Starzinski-Powitz A, Arosh J A 2008 Cyclooxygenase-2 regulates survival, migration and invasion of human endometriotic cells through multiple mechanisms. Endocrinology 149:1180-1189.
12. Ozawa Y, Murakami T, Tamura M, Terada Y, Yaegashi N, Okamura K 2006 A selective cyclooxygenase-2 inhibitor suppresses the growth of endometriosis xenografts via antiangiogenic activity in severe combined immunodeficiency mice. Fertil Steril 86 Suppl 4:1146-1151.
13. Chishima F, Hayakawa S, Sugita K, Kinukawa N, Aleemuzzaman S, Nemoto N, Yamamoto T, Honda M 2002 Increased expression of cyclooxygenase-2 in local lesions of endometriosis patients. Am J Reprod Immunol 48:50-56.
14. Ota H, Igarashi S, Sasaki M, Tanaka T 2001 Distribution of cyclooxygenase-2 in eutopic and ectopic endometrium in endometriosis and adenomyosis. Hum Reprod 16:561-566.
15. De Leon F D, Vijayakumar R, Brown M, Rao C V, Yussman M A, Schultz G 1986 Peritoneal fluid volume, estrogen, progesterone, prostaglandin, and epidermal growth factor concentrations in patients with and without endometriosis. Obstet Gynecol 68:189-194.
16. Cobellis L, Razzi S, De Simone S, Sartini A, Fava A, Danero S, Gioffre W, Mazzini M, Petraglia F 2004 The treatment with a COX-2 specific inhibitor is effective in the management of pain related to endometriosis. Eur J Obstet Gynecol Reprod Biol 116:100-102.
17. Matsuzaki S, Canis M, Darcha C, Dallel R, Okamura K, Mage G 2004 Cyclooxygenase-2 selective inhibitor prevents implantation of eutopic endometrium to ectopic sites in rats. Fertil Steril 82:1609-1615.
18. Hull M L, Prentice A, Wang D Y, Butt R P, Phillips S C, Smith S K, Charnock-Jones D S 2005 Nimesulide, a COX-2 inhibitor, does not reduce lesion size or number in a nude mouse model of endometriosis. Hum Reprod 20:350-358.
19. Matsuzaki S, Canis M 2005 Is the dose to inhibit the COX-2 enzyme in nude mice also adequate in ‘human’ endometrial tissues? Hum Reprod 20:2665; author reply 2665-2666.
20. Attar E, Bulun S E 2006 Aromatase and other steroidogenic genes in endometriosis: translational aspects. Hum Reprod Update 12:49-56.
21. Ebert A D, Bartley J, David M 2005 Aromatase inhibitors and cyclooxygenase-2 (COX-2) inhibitors in endometriosis: new questions—old answers? Eur J Obstet Gynecol Reprod Biol 122:144-150.
22. Attar E, Tokunaga H, Imir G, Yilmaz M B, Redwine D, Putman M, Gurates B, Attar R, Yaegashi N, Hales D B, Bulun S E 2008 Prostaglandin E2 via Steroidogenic Factor-1 Coordinately Regulates Transcription of Steroidogenic Genes Necessary for Estrogen Synthesis in Endometriosis. J Clin Endocrinol Metab.
23. Olivares C, Bilotas M, Buquet R, Borghi M, Sueldo C, Tesone M, Meresman G 2008 Effects of a selective cyclooxygenase-2 inhibitor on endometrial epithelial cells from patients with endometriosis. Hum Reprod 23:2701-2708.
24. Lim H, Paria B C, Das S K, Dinchuk J E, Langenbach R, Trzaskos J M, Dey S K 1997 Multiple female reproductive failures in cyclooxygenase 2-deficient mice. Cell 91:197-208.
25. Cha Y I, DuBois R N 2007 NSAIDs and cancer prevention: targets downstream of COX-2. Annu Rev Med 58:239-252.
26. Narumiya S, Sugimoto Y, Ushikubi F 1999 Prostanoid receptors: structures, properties, and functions. Physiol Rev 79:1193-1226.
27. Pai R, Soreghan B, Szabo I L, Pavelka M, Baatar D, Tarnawski A S 2002 Prostaglandin E2 transactivates EGF receptor: a novel mechanism for promoting colon cancer growth and gastrointestinal hypertrophy. Nat Med 8:289-293.
28. Regan J W 2003 EP2 and EP4 prostanoid receptor signaling. Life Sci 74:143-153.
29. Buchanan F G, Gorden D L, Matta P, Shi Q, Matrisian L M, DuBois R N 2006 Role of beta-arrestin 1 in the metastatic progression of colorectal cancer. Proc Natl Acad Sci USA 103:1492-1497.
30. Castellone M D, Teramoto H, Williams B O, Druey K M, Gutkind J S 2005 Prostaglandin E2 promotes colon cancer cell growth through a Gs-axin-beta-catenin signaling axis. Science 310:1504-1510.
31. Jabbour H N, Sales K J 2004 Prostaglandin receptor signalling and function in human endometrial pathology. Trends Endocrinol Metab 15:398-404.
32. Wang D, Dubois R N 2006 Prostaglandins and cancer. Gut 55:115-122.
33. Eisinger A L, Prescott S M, Jones D A, Stafforini D M 2007 The role of cyclooxygenase-2 and prostaglandins in colon cancer. Prostaglandins Other Lipid Mediat 82:147-154.
34. Clevers H 2006 Colon cancer—understanding how NSAIDs work. N Engl J Med 354:761-763.
35. Harada T, Taniguchi F, Izawa M, Ohama Y, Takenaka Y, Tagashira Y, Ikeda A, Watanabe A, Iwabe T, Terakawa N 2007 Apoptosis and endometriosis. Front Biosci 12:3140-3151.
36. Izawa M, Harada T, Deura I, Taniguchi F, Iwabe T, Terakawa N 2006 Drug-induced apoptosis was markedly attenuated in endometriotic stromal cells. Hum Reprod 21:600-604.
37. Harada T, Kaponis A, Iwabe T, Taniguchi F, Makrydimas G, Sofikitis N, Paschopoulos M, Paraskevaidis E, Terakawa N 2004 Apoptosis in human endometrium and endometriosis. Hum Reprod Update 10:29-38.
38. Arosh J A, Lee J, Rodriguez R, Starzinski-Powitz A, Banu S K 2007 Prostaglandin E2 Signaling in Pathophysiology of Endometriosis in Human. Biology of Reproduction Special Issue.
39. Banu S K, Lee J, Starzinski-Powitz A, Arosh J A 2007 Gene expression profiles and functional characterization of human immortalized endometriotic epithelial and stromal cells. Fertility and Sterlity (In press: PMID 18001719).
40. Coleman R A, Smith W L, Narumiya S 1994 International Union of Pharmacology classification of prostanoid receptors: properties, distribution, and structure of the receptors and their subtypes. Pharmacol Rev 46:205-229.
41. Woodward D F, Pepperl D J, Burkey T H, Regan J W 1995 6-Isopropoxy-9-oxoxanthene-2-carboxylic acid (AH 6809), a human EP2 receptor antagonist. Biochem Pharmacol 50:1731-1733.
42. Crider J Y, Griffin B W, Sharif N A 2000 Endogenous EP4 prostaglandin receptors coupled positively to adenylyl cyclase in Chinese hamster ovary cells: pharmacological characterization. Prostaglandins Leukot Essent Fatty Acids 62:21-26.
43. Jiang X, Wang X 2004 Cytochrome C-mediated apoptosis. Annu Rev Biochem 73:87-106.
44. Adams J M, Cory S 1998 The Bcl-2 protein family: arbiters of cell survival. Science 281:1322-1326.
45. Zha J, Harada H, Yang E, Jockel J, Korsmeyer S J 1996 Serine phosphorylation of death agonist BAD in response to survival factor results in binding to 14-3-3 not BCL-X(L). Cell 87:619-628.
46. Bonni A, Brunet A, West A E, Datta S R, Takasu M A, Greenberg M E 1999 Cell survival promoted by the Ras-MAPK signaling pathway by transcription-dependent and -independent mechanisms. Science 286:1358-1362.
47. Datta S R, Dudek H, Tao X, Masters S, Fu H, Gotoh Y, Greenberg M E 1997 Akt phosphorylation of BAD couples survival signals to the cell-intrinsic death machinery. Cell 91:231-241.
48. Kumar A, Takada Y, Boriek A M, Aggarwal B B 2004 Nuclear factor-kappaB: its role in health and disease. J Mol Med 82:434-448.
49. Grigoryan T, Wend P, Klaus A, Birchmeier W 2008 Deciphering the function of canonical Wnt signals in development and disease: conditional loss- and gain-of-function mutations of beta-catenin in mice. Genes Dev 22:2308-2341.
50. Zandi R, Larsen A B, Andersen P, Stockhausen M T, Poulsen H S 2007 Mechanisms for oncogenic activation of the epidermal growth factor receptor. Cell Signal 19:2013-2023.
51. Ding Q, Xia W, Liu J C, Yang J Y, Lee D F, Xia J, Bartholomeusz G, Li Y, Pan Y, Li Z, Bargou R C, Qin J, Lai C C, Tsai F J, Tsai C H, Hung M C 2005 Erk associates with and primes GSK-3beta for its inactivation resulting in upregulation of beta-catenin. Mol Cell 19:159-170.
52. Nisolle M, Casanas-Roux F, Donnez J 1997 Immunohistochemical analysis of proliferative activity and steroid receptor expression in peritoneal and ovarian endometriosis. Fertil Steril 68:912-919.
53. Milne S A, Perchick G B, Boddy S C, Jabbour H N 2001 Expression, localization, and signaling of PGE(2) and EP2/EP4 receptors in human nonpregnant endometrium across the menstrual cycle. J Clin Endocrinol Metab 86:4453-4459.
54. Nguyen M, Camenisch T, Snouwaert J N, Hicks E, Coffman T M, Anderson P A, Malouf N N, Koller B H 1997 The prostaglandin receptor EP4 triggers remodelling of the cardiovascular system at birth. Nature 390:78-81.
55. Tilley S L, Audoly L P, Hicks E H, Kim H S, Flannery P J, Coffman T M, Koller B H 1999 Reproductive failure and reduced blood pressure in mice lacking the EP2 prostaglandin E2 receptor. J Clin Invest 103:1539-1545.
56. Nisolle M, Casanas-Roux F, Anaf V, Mine J M, Donnez J 1993 Morphometric study of the stromal vascularization in peritoneal endometriosis. Fertil Steril 59:681-684.
57. American Society for Reproductive Medicine 1997 Revised American Society for Reproductive Medicine Classification of Endometriosis: 1996. Fertility and Sterlity 67:817-821.
58. Kim Y A, Kim M R, Lee J H, Kim J Y, Hwang K J, Kim H S, et al. Gonadotropin-releasing hormone agonist reduces aromatase cytochrome P450 and cyclooxygenase-2 in ovarian endometrioma and eutopic endometrium of patients with endometriosis. Gynecol Obstet Invest 2009; 68:73-81.
59. Baek J Y, Hur W, Wang J S, Bae S H, Yoon S K. Selective COX-2 inhibitor, NS-398, suppresses cellular proliferation in human hepatocellular carcinoma cell lines via cell cycle arrest. World J Gastroenterol 2007; 13:1175-81.
60. Cheng J, Imanishi H, Amuro Y, Hada T. NS-398, a selective cyclooxygenase 2 inhibitor, inhibited cell growth and induced cell cycle arrest in human hepatocellular carcinoma cell lines. Int Cancer 2002; 99:755-61.
61. Cheng J, Imanishi H, Liu W, Nakamura H, Morisaki T, Higashino K, et al. Involvement of cell cycle regulatory proteins and MAP kinase signaling pathway in growth inhibition and cell cycle arrest by a selective cyclooxygenase 2 inhibitor, etodolac, in human hepatocellular carcinoma cell lines. Cancer Sci 2004; 95:666-73.
62. Dvory-Sobol H, Cohen-Noyman E, Kazanov D, Figer A, Birkenfeld S, Madar-Shapiro L, et al. Celecoxib leads to G2/M arrest by induction of p21 and down-regulation of cyclin B1 expression in a p53-independent manner. Eur J Cancer 2006; 42:422-6.
63. Kardosh A, Blumenthal M, Wang W J, Chen T C, Schonthal A H. Differential effects of selective COX-2 inhibitors on cell cycle regulation and proliferation of glioblastoma cell lines. Cancer Biol Ther 2004; 3:55-62.
64. Lin H P, Kulp S K, Tseng P H, Yang Y T, Yang C C, Chen C S. Growth inhibitory effects of celecoxib in human umbilical vein endothelial cells are mediated through G1 arrest via multiple signaling mechanisms. Mol Cancer Ther 2004; 3:1671-80.
65. Shin Y K, Park J S, Kim H S, Jun H J, Kim G E, Suh C O, et al. Radiosensitivity enhancement by celecoxib, a cyclooxygenase (COX)-2 selective inhibitor, via COX-2-dependent cell cycle regulation on human cancer cells expressing differential COX-2 levels. Cancer Res 2005; 65:9501-9.
66. Yazawa K, Tsuno N H, Kitayama J, Kawai K, Okaji Y, Asakage M, et al. Selective inhibition of cyclooxygenase (COX)-2 inhibits endothelial cell proliferation by induction of cell cycle arrest. Int J Cancer 2005; 113:541-8.
67. Zhi H, Wang L, Zhang J, Zhou C, Ding F, Luo A, et al. Significance of COX-2 expression in human esophageal squamous cell carcinoma. Carcinogenesis 2006; 27:1214-21.
68. Trifan O C, Hla T. Cyclooxygenase-2 modulates cellular growth and promotes tumorigenesis. J Cell Mol Med 2003; 7:207-228.
69. Cheong E, Ivory K, Doleman J, Parker M L, Rhodes M, Johnson I T. Synthetic and naturally occurring COX-2 inhibitors suppress proliferation in a human oesophageal adenocarcinoma cell line (OE33) by inducing apoptosis and cell cycle arrest. Carcinogenesis 2004; 25:1945-52.
70. Banu S K, Lee J, Speights V O Jr, Starzinski-Powitz A, Arosh J A. Selective inhibition of prostaglandin E2 receptors EP2 and EP4 induces apoptosis of human endometriotic cells through suppression of ERK1/2, AKT, NFkB and b-catenin pathways and activation of intrinsic apoptotic mechanisms. Mol Endocrinol 2009; 23:1291-305.
71. Matsuzaki S, Canis M, Pouly J L, Botchorishvili R, Dechelotte P J, Mage G. Differential expression of genes in eutopic and ectopic endometrium from patients with ovarian endometriosis. Fertil Steril 2006; 86:548-53.
72. Johnson D G, Walker C L. Cyclins and cell cycle checkpoints Annu Rev Pharmacol Toxicol 1999; 39: 295-312.
73. Malumbres M, Barbacid M. Mammalian cyclindependent kinases. Trends Biochem Sci 2005; 30:630-41.
74. Sanchez I, Dynlacht B D. New insights into cyclins, CDKs, and cell cycle control. Semin Cell Dev Biol 2005; 16:311-21.
75. Schwartz G K, Shah M A. Targeting the cell cycle: a new approach to cancer therapy. J Clin Oncol 2005; 23:9408-21.
76. Blundell R A. The biology of p21. Am J Biochem Biotechnol 2006; 2:33-40.
77. Enge M, Bao W, Hedstrom E, Jackson S P, Moumen A, Selivanova G. MDM2-dependent downregulation of p21 and hnRNP K provides a switch between apoptosis and growth arrest induced by pharmacologically activated p53. Cancer Cell 2009; 15:171-83.
78. Attar E, Tokunaga H, Imir G, Yilmaz M B, Redwine D, Putman M, et al. Prostaglandin E2 via steroidogenic factor-1 coordinately regulates transcription of steroidogenic genes necessary for estrogen synthesis in endometriosis. J Clin Endocrinol Metab 2008.
79. Maia H Jr, Maltez A, Studard E, Zausner B, Athayde C, Coutinho E. Effect of the menstrual cycle and oral contraceptives on cyclooxygenase-2 expression in the endometrium. Gynecol Endocrinol 2005; 21:57-61.
80. Smith O P, Jabbour H N, Critchley H O. Cyclooxygenase enzyme expression and E series prostaglandin receptor signalling are enhanced in heavy menstruation. Hum Reprod 2007; 22: 1450-6.
81. Arosh J A, Banu S K, Chapdelaine P, Emond V, Kim J J, MacLaren L A, et al. Molecular cloning and characterization of bovine prostaglandin E2 receptors EP2 and EP4: expression and regulation in endometrium and myometrium during the estrous cycle and early pregnancy. Endocrinology 2003; 144:3076-91.
82. Zeitvogel A, Baumann R, Starzinski-Powitz A. Identification of an invasive, N-cadherin-expressing epithelial cell type in endometriosis using a new cell culture model. Am J Pathol 2001; 159:1839-52.
83. Grund E M, Kagan D, Tran C A, Zeitvogel A, Starzinski-Powitz A, Nataraja S, et al. Tumor necrosis factor alpha regulates inflammatory and mesenchymal responses via mitogen-activated protein kinase kinase, p38, and nuclear factor kappaB in human endometriotic epithelial cells. Mol Pharmacol 2008; 73:1394-404.
84. Wu Y, Starzinski-Powitz A, Guo S W, Trichostatin A, a histone deacetylase inhibitor, attenuates invasiveness and reactivates E-cadherin expression in immortalized endometriotic cells. Reprod Sci 2007; 14:374-82.
85. Wu Y, Starzinski-Powitz A, Guo S W. Prolonged stimulation with tumor necrosis factor-alpha induced partial methylation at PR-B promoter in immortalized epithelial-like endometriotic cells. Feral Steril 2008; 90: 234-7.
86. Bohn W, Wiegers W, Beuttenmuller M, Traub P. Species-specific recognition patterns of monoclonal antibodies directed against vimentin. Exp Cell Res 1992; 201:1-7.
87. Krishna T S, Fenyo D, Kong X P, Gary S, Chait B T, Burgers P, et al. Crystallization of proliferating cell nuclear antigen (PCNA) from Saccharomyces cerevisiae. J Mol Biol 1994; 241:265-8.
88. Maga G, Hubscher U. Proliferating cell nuclear antigen (PCNA): a dancer with many partners. J Cell Sci 2003; 116:3051-60.
89. Nagase H, Woessner J F Jr. Matrix metalloproteinases. J Biol Chem 1999; 274:21491-4.
90. Bruner-Tran K L, Zhang Z, Eisenberg E, Winneker R C, Osteen K G. Down-regulation of endometrial matrix metalloproteinase-3 and -7 expression in vitro and therapeutic regression of experimental endometriosis in vivo by a novel nonsteroidal progesterone receptor agonist, tanaproget. J Clin Endocrinol Metab 2006; 91:1554-60.
91. Curry T E Jr, Osteen K G. The matrix metalloproteinase system: changes, regulation, and impact throughout the ovarian and uterine reproductive cycle. Endocr Rev 2003; 24:428-65.
92. Banu S K, Starzinski-Powitz A, Speights V O, Burghardt R C, Arosh J A 2009 Induction of peritoneal endometriosis in nude mice using human immortalized endometriosis epithelial and stromal cells: A potential experimental tool to study molecular pathogenesis of endometriosis in human. Fertil Steril 91:2199-2209
93. Golding M C, Long C R, Carmell M A, Hannon G J, Westhusin M E 2006 Suppression of prion protein in livestock by RNA interference. Proc Natl Acad Sci USA 103:5285-5290

What is claimed is:

1. A method of treating one or more chronic gynecological diseases in a subject comprising the steps of:
identifying the subject in need of treatment against the one or more chronic gynecological diseases; and
administering a pharmaceutical composition comprising a therapeutically effective amount of one or more selective inhibitors of prostaglandin E2 (PGE₂) receptors sufficient to treat the one or more chronic gynecological diseases.

2. The method of claim 1, wherein the one or more chronic gynecological diseases are selected from endometriosis, dysmenorrhea, dyspareunia, non-cyclic pelvic and abdominal pain, subfertility, infertility, and pelvic cancer.

3. The method of claim 2, wherein the pharmaceutical composition for treating endometriosis prevents a growth, survival, proliferation, migration, and invasion of one or more endometriosis epithelial cells and stromal cells.

4. The method of claim 2, wherein the pharmaceutical composition for treating endometriosis modulates one or more G-protein coupled receptor mediated cell signaling pathways as determined by the activation at least one of the ERK1/2, AKT, NFκB or β-catenin signaling pathways.

5. The method of claim 4, wherein the one or more cell signaling pathways comprise epidermal growth factor receptor (EGFR), nuclear factor kappa B (NFκB), and β-catenin/Wnt.

6. The method of claim 1, wherein the subject is a female subject in an age group of 12 to 50 years.

7. The method of claim 1, further comprising the steps of:
monitoring a progression of the one or more chronic gynecological diseases following the administration of the pharmaceutical composition; and
continuing, terminating or modifying the administration of the pharmaceutical composition based on the progression of the one or more chronic gynecological diseases, wherein the modification comprises an increase or a decrease in a dosage, a frequency or both of the pharmaceutical composition.

8. The method of claim 1, wherein the one or more inhibitors at least partially and selectively inhibit at least one of PGE₂ receptors EP1, EP2, EP3, and EP4.

9. The method of claim 1, wherein the one or more inhibitors at least partially and selectively inhibit at least one of PGE₂ receptors EP2 and EP4.

10. The method of claim 1, wherein the one or more inhibitors are selected from at least one of 6-Isopropoxy-9-oxoxanthene-2-carboxylic acid and (4Z)-7-[(rel-1S,2S,5R)-5-((1,1′-Biphenyl-4-yl)methoxy)-2-(4-morpholinyl)-3-oxocyclopentyl]-4-heptenoic acid, derivatives or salts thereof.

11. The method of claim 1, wherein the one or more inhibitors are administered at concentrations of 10 μM, 25 μM, 50 μM, 75 μM, 100 μM, 250 μM, 500 μM, and 1000 μM.

12. The method of claim 1, wherein the pharmaceutical composition is administered subcutaneously, intravenously, intraperitoneally, orally, intramuscularly, and intravaginally.

13. A pharmaceutical composition for treating one or more chronic gynecological diseases in a subject comprising:
a therapeutically effective amount of one or more selective inhibitors of prostaglandin E2 (PGE₂) receptors sufficient to treat the one or more chronic gynecological diseases optionally dissolved, dispersed or suspended in an aqueous or a non-aqueous solvent; and
one or more optional excipients selected from the group consisting of fillers, diluents, extended or controlled release agents, bulking agents, antiadherents, binders, lubricants, preservatives, and other organoleptic compounds.

14. The composition of claim 13, wherein the pharmaceutical composition is used in the treatment of one or more chronic gynecological diseases selected from endometriosis, dysmenorrhea, dyspareunia, non-cyclic pelvic and abdominal pain, subfertility, infertility, and pelvic cancer.

15. The composition of claim 14, wherein the pharmaceutical composition for treating endometriosis prevents a growth, survival, proliferation, migration, and invasion of one or more endometriosis epithelial cells and stromal cells.

16. The composition of claim 14, wherein the pharmaceutical composition for treating endometriosis modulates one or more G-protein coupled receptor mediated cell signaling pathways as determined by the activation at least one of the ERK1/2, AKT, NFκB or β-catenin signaling pathways.

17. The composition of claim 16, wherein the one or more cell signaling pathways comprise epidermal growth factor receptor (EGFR), nuclear factor kappa B (NFκB), and β-catenin/Wnt.

18. The composition of claim 13, wherein the subject is a female subject in an age group of 12 to 50 years.

19. The composition of claim 13, wherein the one or more inhibitors at least partially and selectively inhibit at least one of PGE₂ receptors EP1, EP2, EP3, and EP4.

20. The composition of claim 13, wherein the one or more inhibitors at least partially and selectively inhibit at least one of PGE₂ receptors EP2 and EP4.

21. The composition of claim 13, wherein the one or more inhibitors are selected from at least one of 6-Isopropoxy-9-oxoxanthene-2-carboxylic acid and (4Z)-7-[(rel-1S,2S,5R)-5-((1,1′-Biphenyl-4-yl)methoxy)-2-(4-morpholinyl)-3-oxocyclopentyl]-4-heptenoic acid, derivatives or salts thereof.

22. The composition of claim 13, wherein the one or more inhibitors are administered at concentrations of 10 μM, 25 μM, 50 μM, 75 μM, 100 μM, 250 μM, 500 μM, and 1000 μM.

23. The composition of claim 13, wherein the pharmaceutical composition is administered subcutaneously, intravenously, intraperitoneally, orally, intramuscularly and intravaginally.

24. A method of treating endometriosis in one or more female subjects in an age group of 12 to 50 years in a subject comprising the steps of:
identifying the subject in need of treatment against endometriosis; and
administering a pharmaceutical composition comprising a therapeutically effective amount of one or more selective inhibitors of prostaglandin E₂ (PGE₂) receptors sufficient to treat the endometriosis.

25. The method of claim 24, further comprising the steps of:
monitoring a progression of the endometriosis following the administration of the pharmaceutical composition; and
continuing, terminating or modifying the administration of the pharmaceutical composition based on the progression of the one or more chronic gynecological diseases, wherein the modification comprises an increase or a decrease in a dosage, a frequency or both of the pharmaceutical composition.

26. The method of claim 24, wherein the one or more inhibitors at least partially and selectively inhibit at least one of PGE₂ receptors EP1, EP2, EP3, and EP4.

27. The method of claim 24, wherein the one or more inhibitors at least partially and selectively inhibit at least one or PGE₂ receptors EP2 and EP4.

28. The method of claim 24, wherein the one or more inhibitors are selected from at least one of 6-Isopropoxy-9-oxoxanthene-2-carboxylic acid and (4Z)-7-[(rel-1S,2S,5R)-5-((1,1′-Biphenyl-4-yl)methoxy)-2-(4-morpholinyl)-3-oxocyclopentyl]-4-heptenoic acid, derivatives or salts thereof.

29. The method of claim 24, wherein the one or more inhibitors are administered at concentrations of 10 μM, 25 μM, 50 μM, 75 μM, 100 μM, 250 μM, 500 μM, and 1000 μM.

30. The method of claim 24, wherein the pharmaceutical composition is administered subcutaneously, intravenously, intraperitoneally, orally, intramuscularly, and intravaginally.

31. The method of claim 24, wherein the pharmaceutical composition for treating endometriosis prevents a growth, survival, proliferation, migration and invasion of one or more endometriosis epithelial cells and stromal cells.

32. The method of claim 24, wherein the pharmaceutical composition for treating endometriosis modulates one or more G-protein coupled receptor mediated cell signaling pathways as determined by the activation at least one of the ERK1/2, AKT, NFκB or β-catenin signaling pathways.

33. The method of claim 32, wherein the one or more cell signaling pathways comprise epidermal growth factor receptor (EGFR), nuclear factor kappa B (NFκB), and β-catenin/Wnt.
